SEQUENCE LISTING (5 sequences)

SEQ ID NO: 1 - protein, 369 aa, Homo sapiens
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 2 - protein, 357 aa, Homo sapiens
MADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 3 - DNA, 2567 bp, Homo sapiens
gcttcctttctcgtgttgttatcgggtagctgcctgctcagaacccacaaagcctgcccctcatcccaggcagagagcaacccagctctttccccagacactgagagctggtggtgcctgctgtcccagggagagttgcatcgccctccacagagcaggcttgcatctgactgacccaccatgacacccacagacttcacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 4 - DNA, 2518 bp, Homo sapiens
gcttcctttctcgtgttgttatcgggtagctgcctgctcagaacccacaaagcctgcccctcatcccaggcagagagcaacccagctctttccccagacactgagagctggtggtgcctgctgtcccagggagagttgcatcgccctccacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 5 - DNA, 66 bp, Artificial
  CCR9-specific shRNA hairpin
accgggccagtggaggtctttgttctgttaatattcatagcagaacaaggaccttcactggctttt

(19) United States
(12) Patent Application Publication
(10) Pub. No.: US 2020/0164068 A1
(43) Pub. Date: May 28, 2020

(54) MODULATORS OF TUMOR IMMUNE RESISTANCE FOR THE TREATMENT OF CANCER

(71) Applicant: DEUTSCHES KREBSFORSCHUNGSZENTRUM STIFTUNG DES ÖFFENTLICHEN RECHTS, Heidelberg (DE)

(72) Inventors: Nisit KHANDELWAL, München (DE); Philipp BECKHOVE, Regensburg (DE); Michael BOUTROS, Heidelberg (DE); Marco BREINIG, Schriesheim (DE); Tillmann MICHELS, Regensburg (DE)

(21) Appl. No.: 16/077,868

(22) PCT Filed: Feb. 16, 2017
(86) PCT No.: PCT/EP2017/053540

§ 371 Date: Aug. 14, 2018

Related U.S. Application Data
(60) Provisional application No. 62/295,598, filed on Feb. 16, 2016.
(60) Provisional application No. 62/295,560, filed on Feb. 16, 2016.

(51) Int. Cl. A61K 39/395; C12N 15/113; G01N 33/574; G01N 33/50; A61K 31/517; A61K 31/4375; A61P 35/00

(52) U.S. Cl. CPC A61K 39/3955; C12N 15/1138; G01N 33/57407; A61P 35/00; A61K 31/517; A61K 31/4375; G01N 33/5011

(57) ABSTRACT

The present invention pertains to novel modulators of tumor resistance against T-cell mediated cytotoxic immune responses. The invention provides antagonists of tumor immune escape mechanisms and methods and other aspects related thereto, and therefore provides novel approaches for treating or aiding a treatment of various cancerous diseases and/or the diagnosis thereof. The invention pertains to both negative and positive regulators of tumor cell resistance and suggests the use inhibitors or activators of these genes for therapeutic purposes. In particular aspects, the invention provides combination therapeutics and/or therapies involving such inhibitors or activators. The invention furthermore provides screening methods for novel cancer therapeutics modulating the action of the identified genes, diagnostic approaches to detect cancer resistance to cytotoxic T-cells as well as pharmaceutical compositions and diagnostic kits, for use with or related to for performing these methods.

The present invention pertains to novel modulators of tumor resistance against T-cell mediated cytotoxic immune responses. The invention provides antagonists of tumor immune escape mechanisms and methods and other aspects related thereto, and therefore provides novel approaches for treating or aiding a treatment of various cancerous diseases and/or the diagnosis thereof. The invention pertains to both negative and positive regulators of tumor cell resistance and suggests the use inhibitors or activators of these genes for therapeutic purposes. In particular aspects, the invention provides combination therapeutics and/or therapies involving such inhibitors or activators. The invention furthermore provides screening methods for novel cancer therapeutics modulating the action of the identified genes, diagnostic approaches to detect cancer resistance to cytotoxic T-cells as well as pharmaceutical compositions and diagnostic kits, for use with or related to for performing these methods.

Peripheral immune tolerance is important to prevent autoimmune disorders. However, tumor cells use immune checkpoints to prevent immune recognition (Zitvogel et al, 2006; Rabinovich et al, 2007). Blocking antibodies against surface-expressed immune-regulatory proteins, such as CTLA4 and PD-L1 (Chambers et al, 2001; Blank et al, 2004), boost anti-tumor immunity and are successfully applied in clinical trials (van Elsas et al, 1999; Weber, 2007; Brahmer et al, 2012; Topalian et al, 2012). Still, treatment unresponsiveness is frequent among patients (Topalian et al, 2012), indicating that other immune-checkpoint pathways may be active. Therefore, successful cancer immunotherapy requires a systematic delineation of the entire immune-regulatory circuit—the ‘immune modulatome’—expressed on tumors (Woo et al, 2012; Berrien-Elliott et al, 2013).

A comprehensive detection of immune-checkpoint molecules has been technically challenging due to the lack of robust high-throughput assays that enable a qualitative and quantitative analysis of heterologous interactions between tumor cells and T cells. Screening strategies before have relied on interferon-gamma (IFN-γ) release as an indicator of anti-tumor NK cell activity (Hill & Martins, 2006; Bellucci et al, 2012). However, IFN-γ secretion alone by immune cells does not always correlate with cellular cytotoxicity (Bachmann et al, 1999; Slifka et al, 1999).

Therefore, there is a need in the art for novel approaches to circumvent tumor immune escape mechanisms. The present invention seeks to provide novel therapeutic compounds, including combination therapeutics and therapies involving such compounds, that are able to strengthen a host's immune response, in particular cytotoxic T cell response, against tumor cells. Furthermore, the invention seeks to provide novel strategies to diagnose tumor resistance to immune response and screening approaches for the identification of compounds that are useful in cancer treatment.

The above problem is first solved by a method for reducing resistance of a tumor cell to an immune response, such as a T cell mediated immune response, the method comprising a step of contacting the tumor cell with a modulator of tumor resistance selected from an
(a) inhibitor or antagonist of CCR9, Growth Hormone Releasing Hormone Receptor (GHRHR), FLJ31393, Frizzled Class Receptor 3 (FZD3), Olfactory Receptor, Family 3, Subfamily A, Member 2 (OR3A2), Chemokine (C-X-C Motif) Ligand 3 (CXCL3), Gonadotropin-Releasing Hormone (Type 2) Receptor 2 (GNRHR2), Interleukin 8 (IL8), 5-Hydroxytryptamine (Serotonin) Receptor 1D (HTR1D), Chemokine (C-C Motif) Ligand 23 (CCL23), Chemokine (C-C Motif) Ligand 2 (CCL2), Purinergic Receptor P2Y, G-Protein Coupled, 11 (P2RY11), Thyrotropin-Releasing Hormone Degrading Enzyme (TRHDE), Chemokine (C-C Motif) Receptor 7 (CCR7), Interleukin 8 Receptor Alpha (IL8RA), G Protein-Coupled Receptor 34 (GPR34), Tachykinin Receptor 2 (TACR2), G protein-coupled receptors 43 (GPR43), Olfactory Receptor, Glucagon (GCG), G Protein-Coupled Receptor Kinase 6 (GRK6), Trace Amine Associated Receptor 8 (TAAR8, also known as TRAR5), or Trace Amine Associated Receptor 9 (TAAR9 also known as TRAR3) (or (i) Olfactory receptor family 2 subfamily J member (20R2J2), Vomeronasal 1 receptor (4VN1R4) or Olfactory receptor family 1 subfamily F member 1 (OR1F1); or (ii) Carcinoembryonic antigen related cell adhesion molecule 6 (CEACAM-6) or B and T lymphocyte associated (CD272)), or
(b) an activator or agonist of Ectonucleotide Pyrophosphatase/Phosphodiesterase 2 (ENPP2), Glutamate Receptor, Metabotropic 6 (GRM6), G Protein-Coupled Receptor Kinase 5 (GRK5), Olfactory Receptor, Family 1, Subfamily G, Member 1 (OR1G1), Chemokine (C-C Motif) Receptor 2 (CCR2), Adrenomedullin receptor (ADMR), Glutamate Receptor, Metabotropic 4 (GRM4), Adhesion G Protein-Coupled Receptor V1 (ADGRV1, also known as MASS1), Chemokine (C-X-C Motif) Ligand 9 (CXCL9), Chemokine (C-X-C Motif) Receptor 3 (CXCR3), or Olfactory Receptor, Family 1, Subfamily D, Member 4 (OR1D4).

In one preferred embodiment the modulator of tumor resistance of the invention is
(a) An inhibitor or antagonist of expression, protein function, or signaling of a protein selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272), as applicable, or
(b) an activator or agonist of the expression, protein function, or signaling of a protein selected from ENPP2, GRM6, GRK5, OR1G1, CCR2, ADMR, GRM4, MASS1, CXCL9, CXCR3, or OR1D4, as applicable.

In a more preferred embodiment, the modulator of tumor resistance of the invention is an inhibitor or antagonist of (a)

In a particular preferred of such embodiment, said inhibitor or antagonist of (a) is an inhibitor or antagonist of FZD3.

In an alternative particular preferred of such embodiment, said inhibitor or antagonist of (a) is an inhibitor or antagonist of TRHDE.

In an alternative particular preferred of such embodiment, said inhibitor or antagonist of (a) is an inhibitor or antagonist of IL8.

In an alternative particular preferred of such embodiment, said inhibitor or antagonist of (a) is an inhibitor or antagonist of OR3A2.

In an alternative particular preferred of such embodiment, said inhibitor or antagonist of (a) is an inhibitor or antagonist of P2RY11.

In an alternative particular preferred of such embodiment, said inhibitor or antagonist of (a) is an inhibitor or antagonist of is CCR9.

In another preferred embodiment, the modulator of tumor resistance of the invention is selected from an inhibitor or antagonist of expression, protein function, or signaling of a protein selected from: (i) OR2J2, VN1R4 or OR1F1; or (ii) CEACAM-6 or CD272.

In a particular preferred of such embodiment, said inhibitor or antagonist of (a) is an inhibitor or antagonist of OR2J2.

In an alternative particular preferred of such embodiment, said inhibitor or antagonist of (a) is an inhibitor or antagonist of CEACAM-6.

In other embodiments, the modulator of tumor resistance is an inhibitor or antagonist of a gene of (a) above and/or its corresponding ligand or receptor (as applicable). In alternative embodiments, the modulator of tumor resistance is an activator or agonist of a gene of (b) above and/or its corresponding ligand or receptor (as applicable). Examples of corresponding ligand/receptor pairs include IL8/ILRA8, CCL2/CCR2 and CXCL9/CXCR3.

Entrez gene identifiers, and other information in respect of these negative or positive modulator-genes of immune resistance are provided in table 1.

The term “antagonist” or “inhibitor” in context of the invention refers to a modulator that, when contacted with a protein of interest, causes a decrease in the magnitude of a certain activity or function of the protein compared to the magnitude of the activity or function observed in the absence of the antagonist or inhibitor. Antagonists include those that block or modulate the biological or enzymatic activity, the expression or signalling cascade of a protein of the invention. Antagonists and inhibitors of CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3, or of: (i) OR2J2, VN1R4 or OR1F1; or (ii) CEACAM-6 or CD272, in each case as applicable, may include, but are not limited to, proteins, nucleic acids, carbohydrates, or any other molecules, that bind to these proteins, or that interact with the respective gene loci or gene products such as their mRNA.

In another more preferred embodiment, the modulator of tumor resistance of the invention is an activator or agonist of (b).

In a particular preferred of such embodiment, said activator or agonist of (b) is an activator or agonist of one or other member of the receptor/ligand pair CXCL9 or CXCR3.

In another particular preferred of such embodiment, said activator or agonist of (b) is an activator or agonist of GRM4.

In another particular preferred of such embodiment, said activator or agonist of (b) is an activator or agonist of GRK5.

In another particular preferred of such embodiment, said activator or agonist of (b) is an activator or agonist of CCR2.

The term “agonist” refers to a modulator that, when contacted with a protein of interest, causes an increase in the magnitude of a certain activity, expression or function of the protein compared to the magnitude of the activity or function observed in the absence of the agonist.

Particular agonists of ENPP2, GRM6, GRK5, OR1G1, CCR2, ADMR, GRM4, MASS1, CXCL9, CXCR3, or OR1D4 may include, but are not limited to polypeptides, nucleic acids, carbohydrates, or any other molecules that bind to the respective protein or their encoding gene or gene products such as mRNA.

The method of the invention preferably comprises a step of contacting the tumor cell with said modulator (eg, the inhibitor/antagonist or activator/agonist, as applicable, of the respective gene) of tumor resistance as described herein.

The tumor cell is in a preferred embodiment characterized by a detectable cell surface expression of CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3, or of (i) OR2J2, VN1R4 or OR1F1; or (ii) CEACAM-6 or CD272, in each case as applicable, before the step of contacting the tumor cell with the corresponding modulator of tumor resistance. This is the case for the modulators (a), which inhibit the expression and/or function of their respective target proteins. In the case of the modulators (b), which are activators of the respective proteins, the tumor cell may already have a base level of protein expression. However, the invention also works in absence of any detectable expression of ENPP2, GRM6, GRK5, OR1G1, CCR2, ADMR, GRM4, MASS1, CXCL9, CXCR3, or OR1D4, in each case as applicable. In this event, the step of contacting of the tumor cell with the modulator (b) induces expression of the respective target proteins, and thereby tumor cell resistance to immune responses are antagonized.

Yet a further aspect of the invention pertains to a method for treating a tumor disease in a patient, wherein said tumor disease is characterized by a resistance of said tumor against immune responses, the method comprising a step of
(a) Inhibiting or antagonizing in said patient CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272), or
(b) Activating or agonizing ENPP2, GRM6, GRK5, OR1G1, CCR2, ADMR, GRM4, MASS1, CXCL9, CXCR3, or OR1D4.

The invention also provides a method for aiding a patient's immune response against a tumor disease comprising a step of
(a) Inhibiting or antagonizing in said patient CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272), or
(b) Activating or agonizing ENPP2, GRM6, GRK5, OR1G1, CCR2, ADMR, GRM4, MASS1, CXCL9, CXCR3, or OR1D4.

The methods of the invention may comprise a step of administering to said patient a therapeutically effective amount of a modulator of tumor resistance selected from an
(a) inhibitor or antagonist of CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272), in each case as applicable, or
(b) an activator or agonist of ENPP2, GRM6, GRK5, OR1G1, CCR2, ADMR, GRM4, MASS1, CXCL9, CXCR3, or OR1D4, as applicable.

Preferred modulators of the invention are selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct and/or guide RNA/DNA (gRNA/gDNA), a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

A tumor cell, tumor or tumor disease in context of the invention is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia.

In certain embodiments of the above described methods not only a single modulator but also combinations of modulators may be used to treat the tumor disease. In this aspect, the invention pertains to a method for treating a tumor disease, which is characterized by a resistance against immune responses. The method comprises a step administering to a patient a combination comprising a modulator of tumor resistance selected from
(a) inhibitor or antagonist of CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272), and/or
(b) an activator or agonist of ENPP2, GRM6, GRK5, OR1G1, CCR2, ADMR, GRM4, MASS1, CXCL9, CXCR3, or OR1D4;
   and at least one additional compound effective in the treatment of the tumor disease. The at least one additional compound effective in the treatment of the tumor disease may be selected from any cancer therapeutic known in the art to be effective for the therapy of the given cancer disease. Preferred embodiments of this aspect pertain to combinations comprising multiple of the above mentioned modulators of tumor resistance of the invention. The combinations may include 2, 3, 4, 5, 6 or more modulators of the invention. For example, one might combine one or more inhibitor or antagonist (a), or one or more activator or agonist (b), or one or more inhibitor or antagonist (a) with or one or more activator or agonist (b).

In alternative embodiments of such combinations, the invention may relate to a combination comprising (a) and (b) or (a) and (c) or (a), (b) and (c), wherein
(a) Is an inhibitor or antagonist of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9 (or of (i) OR2J2, VN1R4 or OR1F1; or (ii) CEACAM-6 or CD272),
(b) Is an inhibitor or antagonist of PI3K-Akt signaling and/or an inhibitor or antagonist of p70S6 kinase signaling, and
(c) Is an activator or agonist of ERK1/2 signaling and/or an activator or agonist of JNK signaling.

Another aspect of the invention then pertains to a method for identifying a (therapeutic) compound suitable for the treatment of a tumor disease, the method comprising the steps of
(a) Providing a first cell expressing a protein on the cellular surface, wherein the protein is selected from: (x) CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272); or is selected from (y) ENPP2, GRM6, GRK5, OR1G1, CCR2, ADMR, GRM4, MASS1, CXCL9, CXCR3, or OR1D4,
(b) Contacting said first cell with a candidate compound,
(c) And/or, contacting subsequent to step (b) said first cell with a cytotoxic T-lymphocyte (CTL), and
(d) Determining subsequent to step (b) and/or (c) protein expression/function in said first cell, wherein a differential protein expression in said first cell contacted with the candidate compound compared to said first cell not contacted with said candidate compound indicates that the candidate compound is a (therapeutic) compound suitable for the treatment of a tumor disease; and/or
(e) Determining subsequent to step (c) cytotoxicity of said CTL against said first cell, wherein an enhanced cytotoxicity of said CTL against said first cell contacted with the candidate compound compared to the cytotoxicity of said CTL against said first cell not contacted with the candidate compound indicates that the candidate compound is a (therapeutic) compound suitable for the treatment of a tumor disease.

The screening method of may be used for the identification of a modulator for use in context of the invention. The screening may be performed only on basis of protein expression of the proteins of the invention, but alternatively may include direct protein function assays. The assay to detect protein function will greatly vary depending on the candidate protein that is assayed. For example, the function of a kinase may be monitored by respective phosphorylation assays. The present invention shall not be restricted to a specific type of assay to assess protein expression and/or function. The person of skill knows respective assays to put the screening method into practise.

In one embodiment the screening method is preferred wherein a reduced protein expression/function of a protein selected from: (x) CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, or TRAR3 (or (i) OR2J2, VN1R4 or OR1F1; or (ii) CEACAM-6 or CD272) in said first cell contacted with the candidate compound compared to said first cell not contacted with said candidate compound indicates that the candidate compound is a (therapeutic) compound suitable for the treatment of a tumor disease.

Alternatively or additionally, the screening method is preferred wherein an increased protein expression/function of a protein selected from: (y) ENPP2, GRM6, GRK5, OR1G1, CCR2, ADMR, GRM4, MASS1, CXCL9, CXCR3, or OR1D4 in said first cell contacted with the candidate compound compared to said first cell not contacted with said candidate compound indicates that the candidate compound is a (therapeutic) compound suitable for the treatment of a tumor disease.

The screening method may, in certain embodiments, include those wherein said tumor disease or tumor derived cell is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia (or a tumor cell derived therefrom).

In the screening method said first cell may be a cell resistant to cytotoxicity mediated by T-lymphocytes, and preferably is a tumor-derived cell.

The candidate compound is selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct and/or guide RNA/DNA (gRNA/gDNA), a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

Finally there is provided a method for diagnosing in a patient a resistance of a tumor disease against immune responses, the method comprising a step of
(a) determining expression of a protein or mRNA selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272) in a tumor cell from the tumor of the patient, wherein a detectable or increased expression of any one of the proteins in the tumor cell compared to a negative control is indicative for a resistance of the tumor disease against T cell mediated immune responses; or
(b) determining expression of a protein or mRNA selected from ENPP2, GRM6, GRK5, OR1G1, CCR2, ADMR, GRM4, MASS1, CXCL9, CXCR3, or OR1D4 in a tumor cell from the tumor of the patient, wherein a reduced expression of any one of the proteins in the tumor cell compared to a negative control is indicative for a resistance of the tumor disease against T cell mediated immune responses.

In certain embodiments, such diagnostic method may comprise a preceding step of obtaining a tumor cell from the patient.

In further embodiments of such diagnostic method, said expression may be a cell surface expression of said protein of (a) or (b) on the tumor cell.

In yet further embodiments of such diagnostic method, said tumor disease may be selected from a liquid or solid tumor, such as breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia.

In the following, the specific embodiments of the present invention will, in certain embodiments, be further illustrated for one specific example of modulators of CCR9. However, such descriptions are preferred aspects and embodiments of the invention and shall equally apply for the above described inhibitors and antagonists of the other proteins identified in the present screen. Furthermore, such descriptions shall be used to envision corresponding embodiments for activators and agonists of the positive regulators identified in the screen.

Preferably are methods wherein the resistance of a tumor disease against immune responses is a resistance of the tumor disease against CTL mediated immune responses.

Thus, the above problem is solved in a another aspect by a method for reducing resistance of a tumor cell to an immune response, such as a T cell mediated immune response, the method comprising a step of contacting the tumor cell with a modulator of tumor resistance selected from an inhibitor or antagonist of CCR9. Preferred aspects of the invention pertain to the use of an inhibitor or antagonist of CCR9, eg CCR9 protein or mRNA.

The term “C-C chemokine receptor type 9”, or short “CCR9”, refers to a chemokine receptor involved in immune cell trafficking (Kunkel et al, 2000; Uehara et al, 2002) and which is expressed on tolerogenic plasmacytoid dendritic cells (Hadeiba et al, 2008). CCR9 was first identified from its nucleic acid sequence as an orphan putative CC chemokine receptor and then originally designated as “GPR-9-6” (eg submitted 16 Jan. 1996 as GenBank U45982.1). Three groups working at around the same time, independently identified that the ligand for GPR-9-6 was thymus expressed chemokine (TECK), such ligand since designated “CCL25” (Zaballos et al, 1999; J Imm 162:5671. Youn et al, 1999; Blood 94:2533. Zabel et al, 1999; J Exp Med 190:1241). CCR9 has been speculated as putative therapeutic target for a variety of uses and indications (WO 2000/021987; WO 2000/022129; WO 2000/053635; WO 2001/77172; WO 2003/095967), including for certain cancers (WO 2004,045526; WO 2009/018170; WO 2012/082742; WO 2012/082752; WO 2015/075269; Tu et al, 2016; J Hemat Onc 9:10).

CCR9 is a seven transmembrane domain G protein coupled receptor-like protein shown to specifically bind and recognize C-C Motif Chemokine Ligand 25 (CCL25). The CCR9 gene is mapped to the chemokine receptor gene cluster region on human chromosome 3: 45,886,504-45,903,177 forward strand GRCh38:CM000665.2 (Ensembl gene Id: ENSG00000173585 referring to Ensembl release 87—December 2016). There are two alternatively spliced transcript variants known. Further synonyms of the gene include C-C Motif Chemokine Receptor 9, Chemokine (C-C Motif) Receptor, CC-CKR-9, GPR-9-6, GPR28, C-C Chemokine Receptor Type 9, G Protein-Coupled Receptor 28, G-Protein Coupled Receptor 28, CDw199 Antigen, C-C CKR-9, CDw199, and CCR-9. The gene/protein is annotated in various databases, amongst others with the following identifiers: HGNC: 1610, Entrez Gene: 10803, OMIM: 604738, UniProtKB: P51686. The amino acid sequence of human CCR9 isoform 1 is provided in SEQ ID NO: 1. The amino acid sequence of human CCR9-isoform 2 differs from the CCR9-isoform 1 in that amino acids 1-12 are missing. The sequence of isoform 2 is provided in SEQ ID NO: 2. The mRNA of human CCR9 isoforms 1 and 2 are shown as cDNA sequences in SEQ ID NO: 3 and 4. CCR9 orthologs are found in many vertebrates from fish to mammals and primates. Many paralogs of CCR9 are known and can be found in the C-C motif chemokine receptor family (CXCR6, CCR7, CCR1, CCR3, CCR4 etc.). The term CCR9 in some embodiments is used to refer to such human isoform 1 and/or human isoform 2, and in other embodiments may refer to variants (such as fragments) thereof, in particular functional fragments or variants thereof.

A “functional variant” or “functional fragment” of CCR9 is a variant or fragment of the protein of CCR9 that provides, possesses and/or maintains one or more of the herein described functions/activities of the non-variant protein of human CCR9. For example, such functional variant may bind one or more of the same chemostimuli as CCR9proteinCCR9 protein, may signal the same G protein-coupled adenylyl cyclase cascade as the CCR9 protein and/or may be coupled to one or more of the same Gas and Gα15 G proteins as CCR9 protein, such as having the same, essentially the same or similar specificity and/or function as a receptor as CCR9 protein. In other embodiments, such a functional variant or function fragment may possess other activities than those possessed by the non-variant CCR9 protein, as long as, preferably, it provides, possesses and/or maintains at least one function/activity that is the same, essentially the same or similar as human CCR9 protein. In more preferred embodiments, a functional variant of CCR9 protein may act as an immune checkpoint inhibitor, such as by inhibiting cell-based immune response to a cancer cell that expresses such functional variant.

The terms “CCR9-protein” or “protein of CCR9” as used in context of the herein disclosed invention shall pertain to a protein (such as a full-length protein, fusion protein or partial protein) comprising a CCR9 sequence, such as as shown in SEQ ID NO: 1 or 2. The terms shall also refer to a protein comprising a CCR9 sequence, such as the amino acid sequence according to SEQ ID NO: 1 or 2, with any protein modifications. Such protein modifications preferably do not alter the amino acid sequence of the polypeptide chain, but constitute a functional group, which is conjugated to the basic amino acid polymer chain. Protein modifications in context of the invention may be selected from a conjugation of additional amino acid sequences to the CCR9 amino acid chain, such as ubiquitination, sumolation, neddylation, or similar small protein conjugates. Other protein modifications include, but are not limited to, glycosylation, methylation, lipid-conjugation, or other natural or artificial post-translational modifications known to the skilled person. The terms “protein of a variant of CCR9” and the like, shall have the corresponding meaning with respect to a variant of CCR9.

The terms “CCR9-mRNA” or “mRNA of CCR9” as used in context of the herein disclosed invention shall pertain to a messenger ribonucleic acid (such as a full-length mRNA, fusion mRNA or partial mRNA, and/or splice-variants thereof) comprising a region encoding for a CCR9 protein, such as an amino acid sequence as shown in SEQ ID NO: 1 or 2. The terms shall also refer to an mRNA comprising a region encoding for a CCR9 protein, such as the amino acid sequence according to SEQ ID NO: 1 or 2, with any codon or nucleotide modifications. Such modifications preferably would not alter the amino acid sequence of the encoded polypeptide chain. The terms “mRNA of a variant of CCR9” and the like, shall have the corresponding meaning with respect to a variant of CCR9. Preferred CCR9 mRNA of the invention comprises an RNA sequence corresponding to the cDNA sequence shown in SEQ ID NO: 3 or 4.

A variant of CCR9 is, in some embodiments, a protein comprising an amino acid sequence having at least 60%, 70%, 80%, 90%, preferably at least 80% such as at least 90% sequence identity to SEQ ID NO: 1 or 2, and most preferably at least 95% (such as at least 98%) sequence identity to SEQ ID NO: 1 or 2 (the human CCR9 amino acid sequence of isoform 1 and 2). In one preferred embodiment of the invention, the variant of CCR9 comprises an amino acid sequence with at least 80% sequence identity to the amino acid sequence shown in SEQ ID NO: 1 or 2. A variant of CCR9 is, in some other embodiments, a protein comprising an amino acid sequence of SEQ ID NO: 1 or 2 wherein between one and about ten amino acids comprised therein have been substituted with another amino acid or analog thereof, preferably a neutral amino acid substitution. In certain of such embodiments, no more than one, two, three, four or five (preferably, no more than two, such as no more than one) amino acid is so substituted. Such amino acid changes, may be present in a population as natural polymorphism, or may be generated by recombinant technologies so as to investigate functional and/or binding properties of the regions of CCR9 protein.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of CCR9 to another protein/gene, the percentage identity can be determined by the Blast searches supported at the NCBI web site; in particular for amino acid identity, those using BLASTP with the following parameters: Expected threshold 10; Word size: 6; Matrix: BLOSUM62; Gap Costs: Existence: 11, Extension: 1; Neighboring words threshold: 11; Compositional adjustments: Conditional compositional score matrix adjustment.

A variant of CCR9 can, in certain embodiments, comprise a fragment of CCR9, for example a polypeptide that consists of one or more extracellular domains (or regions thereof) of CCR9 without one or other (or any other) extracellular, transmembrane or intracellular domains of CCR9.

An inhibitor of CCR9 expression or an inhibitor of CCR9-T-cell interaction in context of the invention may be any compound that impairs or interferes with the expression of CCR9 (such as the expression of CCR9 mRNA and/or protein) or that impairs or interferes with the function of CCR9 as a mediator of T-cell and tumor cell interaction or that impairs or interferes with the signalling through a pathway mediated by CCR9. Preferred are, in context of the invention, such inhibitors that inhibit CCR9 expression or function specifically and selectively in tumor cells. In one embodiment, the inhibitor of CCR9 expression or inhibitor of CCR9-T-cell interaction or inhibitor of CCR9 signalling may inhibit CCR9 via a direct interaction with the CCR9 polypeptide, its RNA transcript or its coding genetic locus. Such inhibitors in context of the invention will be referred to as “CCR9 inhibitors or antagonists”, or similar expressions. In other embodiments, the invention also includes inhibitors of CCR9 expression or inhibitors of CCR9-T-cell interaction or inhibitors of CCR9 signalling that interact with other components of the CCR9 immune modulatory function as disclosed herein.

In other aspects, the invention provides an inhibitor or antagonist of any of the negative regulator genes the subject matter hereof, such as an inhibitor of CCR9 expression or inhibitor of CCR9-T-cell interaction or inhibitor of CCR9 signalling, that reduces the resistance of a tumor cell to an immune response, such as a T cell mediated immune response; and, in yet other aspects the invention provides an activator or agonist of any of the positive regulator genes the subject matter hereof.

The herein described and disclosed modulators of immune resistance are preferably for use in medicine. In particular embodiment thereof for use in the treatment of a tumor disease of a subject, such as a tumor disease that is characterized by resistance to such immune response and/or is characterised by expression of one or more of the negative regulator genes the subject matter hereof, such as CCR9, and/or by reduced expression of one or more of the positive regulator genes the subject matter hereof. Exemplary such tumor diseases are described elsewhere herein.

In context of the present invention the term “subject” or “patient” preferably refers to a mammal such as a mouse, rat, guinea pig, rabbit, cat, dog, monkey, or, preferably a human, for example a human patient. The subject of the invention may be at danger of suffering from a cancer or tumor disease, or suffer from a cancer or tumor disease, preferably wherein the tumor disease is a tumor having a resistance to the host's (the subject's) immune system, most preferably to cytotoxic T-cell responses. A more detailed description of medical indications relevant in context of the invention is provided elsewhere herein.

The term “resistance” refers to an acquired or natural resistance of a tumor or cancer to a subject's (eg a patient's) own immune response. Therefore, a resistant tumor or tumor cell is more likely to escape and survive humoral and/or cellular immune defense mechanisms in a subject having the tumor or cancer. A treatment of tumor resistance in context of the invention shall be effective if compared to a non-treated control, the tumor or tumor cell becomes more sensitive to an immune response—that is will be more likely to be identified and neutralized by a subject's (eg a patient's) immune system.

In a preferred embodiment of the invention tumor resistance is a tumor resistance to a cytotoxic T lymphocyte (CTL) response against cancer (i.e., the tumor or tumor cell being nonresponsive to, or having reduced or limited response to a CTL). In particular embodiments, the CTL is one capable of recognising the tumor or tumor cell. In this case the tumor cell shows a reduced sensitivity when contacted with a CTL specific for that tumor cell, for example to 90% cytotoxic response, preferably 80%, 70%, 60%, 50% or more preferably 40%, 30%, 20% or even less. In this case, 100% would denote the state wherein the CTL can kill all of the cells in a cancer sample. The reduction in response can be measured by comparing with the same cancer sample before the resistance is acquired, or by comparing with a different (control) cancer sample that is known to have no resistance to the CTL. In some preferred embodiments, the different (control) cancer sample is a sample of tumor cells having no, or no detectable cell surface expression of CCR9. On the other hand, the treatments of the present invention include the sensitization of tumor cells against CTL and therefore to decrease tumor cell resistance. A decrease of tumor cell resistance against CTL is preferably a significant increase of CTL (cyto-) toxicity, preferably a 10% increase, more preferably 20%, 30%, 40%, 50%, 60%, 70%, 80% or more, even more preferably 2 fold increase, 3 fold, 4 fold, 5 fold or more. Resistance or sensitivity of a tumor cell when contacted with a CTL may be investigated using the methods disclosed herein, such as in Example 1.

The inhibitor of (a) (such as CCR9 expression or an inhibitor of CCR9-T-cell interaction or inhibitor of CCR9 signalling) and/or the activator of (b), in each case of the invention, is in some embodiments selected from a compound having an inhibitory activity towards the applicable modulating gene the subject matter of the present invention (eg CCR9) and which is a polypeptide, peptide, glycoprotein, a peptidomimetic, an antibody or antibody-like molecules; a nucleic acid such as a DNA or RNA, for example an antisense DNA or RNA, a ribozyme, an RNA or DNA aptamer, siRNA and the like, including variants or derivatives thereof such as a peptide nucleic acid (PNA); a targeted gene editing construct, such as a CRISPR/Cas9 construct and/or guide RNA/DNA (gRNA/gDNA); a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds. Inhibitors or antagonists of the negative regulator genes the subject matter hereof are, preferably, an antigen binding construct, such as an antibody (or derivatives thereof), or a nucleic acid molecule, such as an inhibitory nucleic acid molecule.

As used herein, the terms “inhibitor of CCR9 expression” or “inhibitor of CCR9-T-cell interaction” or “inhibitor of CCR9 signalling” (and the like) means a substance that affects a decrease in the amount or rate of CCR9 expression or activity as a mediator of intermolecular interaction or activity as a mediator of inter-molecular pathway signalling, respectively. Such a substance can act directly, for example, by binding to CCR9 and decreasing the amount or rate of CCR9 expression. An “inhibitor of CCR9-T-cell interaction” may be any molecule that directly interacts with CCR9 and impairs CCR9 mediated binding to T-cells, or to T-cell surface molecules. An “inhibitor of CCR9 signalling” may be any molecule that directly interacts with CCR9 and impairs CCR9 mediated signalling through a signal transduction pathway associated therewith. A CCR9 antagonist or inhibitor can also decrease the amount or rate of CCR9 expression or activity, for example, by binding to CCR9 in such a way as to reduce or prevent interaction of CCR9 with its substrate on the surface of a T-cell; by binding to CCR9 and modifying it, such as by removal or addition of a moiety, or altering its three-dimensional conformation; and by binding to CCR9 and reducing its stability or conformational integrity. A CCR9 antagonist or inhibitor can also act indirectly, for example, by binding to a regulatory molecule or gene region so as to modulate regulatory protein or gene region function and affect a decrease in the amount or rate of CCR9 expression or activity. Thus, a CCR9 inhibitor or antagonist can act by any mechanisms that result in a decrease in the amount or rate of CCR9 expression or activity.

In certain embodiments, the inhibitor or antagonist of CCR9 does not comprise pertussis toxin (PTX). In other related embodiments, the inhibitor or antagonist of CCR9 does not comprise a (non-specific) G_{αi} inhibitor. In further related embodiments, the inhibitor or antagonist of CCR9 does not inhibit the same signalling pathway as PTX and/or a (non-specific) G_{αi} inhibitor.

C-C chemokine ligand 25 (CCL25, also known as TECK: Entrez ID: 6370; Location: Chromosome 19: 8,052,767-8,062,650 forward strand, GRCh38:CM000681.2; Human CCDS set: CCDS12194.1, CCDS56080.1; UniProtKB identifiers: 015444; Ensembl version: ENSG00000131142.13) is the only known interacting partner and ligand for CCR9. In other certain embodiments, the inhibitor or antagonist of CCR9 does not inhibit or antagonise CCL25 production by the tumor cell and/or inhibit or antagonise CCL25 binding to CCR9 and/or does not inhibit or antagonise CCL25-mediated signalling or function of CCR9. In particular of such embodiments, the inhibitor or antagonist of CCR9 does not inhibit or antagonise migration or chemotaxis of ccr9+ cells (eg, ccr9+ lymphocytes and/or thymocytes). For example, the inhibitor or antagonist of CCR9 may, in such embodiments, be characterised as one that reduces resistance of a tumor cell to an immune response without reducing (eg, maintaining): (x) CCL25 production by the tumor cell (for example, as determined using a method analogous to Example 3); and/or (y) CCL25-mediated chemotaxis of ccr9+ cells (eg, ccr9+ lymphocytes and/or thymocytes).

In one particular embodiment, the inhibitor or antagonist of CCR9 is an inhibitor of CCR9 expression or an inhibitor of CCR9 signaling or an inhibitor of CCR9-T-cell interaction.

An inhibitor of CCR9 expression or an inhibitor of CCR9-T-cell interaction or an inhibitor of CCR9 signalling can be, for example, a naturally or non-naturally occurring macromolecule, such as a polypeptide, peptide, peptidomimetic, nucleic acid, carbohydrate or lipid.

Preferably, a CCR9 antagonist or inhibitor of the invention is isolated. The term “isolated” as used herein in the context of a polypeptide, such as an antigen binding construct, refers to a polypeptide that is purified from proteins or polypeptides or other contaminants that would interfere with its therapeutic, diagnostic, prophylactic, research or other use. Such a polypeptide may be a recombinant, synthetic or modified (non-natural). The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. In this context, a “recombinant” protein/polypeptide or nucleic acid is one made using recombinant techniques. Methods and techniques for the production of recombinant nucleic acids and proteins are well known in the art.

Antigen Binding Constructs

As described above, the inhibitors or antagonists of the negative regulators the subject mater of the present invention (such as CCR9) are in one embodiment, preferably, an antigen binding construct, such as an antibody (or derivatives thereof), More preferably, when such eg CCR9 inhibitor or antagonist is an antigen binding construct, then such antigen binding construct binds to, such as specifically binds to such protein.

The term “antigen binding construct” includes all varieties of antibodies and T cell receptor (TCR) derived polypeptides, which comprise an epitope binding domain, including binding fragments thereof. Further included are constructs that include 1, 2, 3, 4, 5, and/or 6 Complementary Determining Region (CDR)s, the main regions mediating antibody or TCR binding ability and specificity to a given antigenic epitope. In some embodiments, these CDRs can be distributed between their appropriate framework regions in a typical antibody or TCR variable domain. In some embodiments, the CDRs can be within a single peptide chain in others they are located in two or more peptide chains (heavy/light or alpha/beta respectively). In some embodiments, the two or more peptides are covalently linked together, for example via disulfide bonds. In some embodiments, they can be linked via a linking molecule or moiety. In some embodiments, the antigen binding proteins are non-covalent, such as a diabody and a monovalent scFv. Unless otherwise denoted herein, the antigen binding constructs described herein bind to a CCR9 protein, as described in detail herein above. Preferred embodiments of the invention pertain to antibodies, or antibody derived polypeptides, as antigen binding constructs of the invention.

A CCR9 antagonist or inhibitor further can be an antibody, or antigen-binding fragment thereof, such as a monoclonal antibody, humanized antibody, chimeric antibody, minibody, bifunctional anti-body, single chain antibody (scFv), variable region fragment (Fv or Fd), Fab or F(ab)2. A CCR9 antagonist or inhibitor can also be polyclonal antibodies specific for CCR9. A CCR9 antagonist or inhibitor further can be a partially or completely synthetic derivative, analog or mimetic of a naturally occurring macromolecule, or a small organic or inorganic molecule.

An inhibitor of CCR9 expression or an inhibitor of CCR9-T-cell interaction or an inhibitor of CCR9 signalling that is an antibody can be, for example, an antibody that binds to CCR9 and inhibits interaction of a compound expressed by a T-cell with CCR9, or alters the activity of a molecule that regulates CCR9 expression or activity, such that the amount or rate of CCR9 expression or activity is decreased. An antibody useful in a method of the invention can be a naturally occurring antibody, including monoclonal or polyclonal antibodies or fragments thereof, or a non-naturally occurring antibody, including but not limited to a single chain antibody, chimeric antibody, bifunctional antibody, complementarity determining region-grafted (CDR-grafted) antibody and humanized antibody or an antigen-binding fragment thereof.

As mentioned earlier, preferred antigen binding constructs are antibodies and antibody-like constructs. The term “antibody” includes, but is not limited to, genetically engineered or otherwise modified forms of immunoglobulins, such as intrabodies, chimeric antibodies, fully human antibodies, humanized antibodies (e.g. generated by “CDR-grafting”), antibody fragments, and heteroconjugate antibodies (e.g., bispecific antibodies, diabodies, triabodies, tetrabodies, etc.). The term “antibody” includes cys-diabodies and minibodies. Thus, each and every embodiment provided herein in regard to “antibodies”, or “antibody like constructs” is also envisioned as, bi-specific antibodies, diabodies, scFv fragments, chimeric antibody receptor (CAR) constructs, diabody and/or minibody embodiments, unless explicitly denoted otherwise. The term “antibody” includes a polypeptide of the immunoglobulin family or a polypeptide comprising fragments of an immunoglobulin that is capable of non-covalently, reversibly, and in a specific manner binding a corresponding antigen, preferably CCR9 protein as disclosed herein. An exemplary antibody structural unit comprises a tetramer. In some embodiments, a full length antibody can be composed of two identical pairs of polypeptide chains, each pair having one “light” and one “heavy” chain (connected through a disulfide bond). Antibody structure and isotypes are well known to the skilled artisan (for example from Janeway's Immunobiology, 9^{th} edition, 2016).

The recognized immunoglobulin genes of mammals include the kappa, lambda, alpha, gamma, delta, epsilon, and mu constant region genes, as well as the myriad immunoglobulin variable region genes (for more information on immunoglobulin genes see the international ImMunoGeneTics information System®, Lefranc M-P et al, Nucleic Acids Res. 2015 January; 43(Database issue):D413-22; and http://www.imgt.org/). For full-length chains, the light chains are classified as either kappa or lambda. For full-length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively. As used in this invention, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (e.g., F(ab′)2) with the same, essentially the same or similar binding specificity. In some embodiments, the antibody binds specifically to protein of CCR9. Preferred antigen binding constructs according to the invention include an antibody heavy chain, preferably the variable domain thereof, or an antigen binding fragment thereof, and/or an antibody light chain, preferably the variable domain thereof, or an antigen binding fragment thereof. In more preferred embodiments of the invention, the antigen binding fragment binds (such as specifically) to protein of CCR9, and in most preferred embodiments wherein such antigen binding fragment inhibits the expression, function and/or stability of CCR9.

In some embodiments of the invention, the (isolated) antigen binding construct comprises the sequences of an antibody heavy chain variable region CDR1, CDR2, and CDR3; and/or the sequences of an antibody light chain variable region CDR1, CDR2, and CDR3.

In some embodiments the (isolated) antigen binding construct of the invention may comprise in at least one, preferably all, polypeptide chains, antibody constant domain sequences. The origin of the constant domain sequence may be selected from a mouse, rat, donkey, rabbit or human antibody constant domain sequence. The selection of the constant domain is dependent on the indented use of the antigen binding construct of the invention. In some embodiments of the invention the antigen binding construct is chimerized, optionally is humanized or murinized.

A preferred embodiment of the invention pertains to a monoclonal antibody as an (isolated) antigen binding construct. An antibody of the invention may be an IgG type antibody, for example having any of the IgG isotypes.

An inhibitor of CCR9 that is an antibody can be, for example, an antibody that binds to CCR9, and modulates, such as inhibits, CCR9 activity or function, or alters the activity of a molecule that regulates CCR9, expression or activity, such that the amount or rate of function of CCR9, or its expression or stability is altered, such as decreased. An antibody useful in a method of the invention can be a naturally occurring antibody format, including monoclonal or polyclonal antibodies or fragments thereof, or a non-naturally occurring antibody format, including but not limited to a single chain antibody, chimeric antibody, bifunctional antibody, complementarity determining region-grafted (CDR-grafted) antibody, CAR, and humanized antibody or an antigen-binding fragment thereof.

In particular embodiments of the invention, the antigen binding construct, such as an antibody, is non-natural and/or is not a product of nature. In one of such embodiments, the antigen binding construct may be a non-natural antigen binding construct, such as a synthetic, modified or recombinant antigen binding construct. In particular, an antigen binding construct of the invention may contain at least one amino acid substitution (or deletion) modification (such as 2, 3, 4, 5, 6, 7, 8, 9, 10 or more than 10 such modifications, in particular between 1 and about 5 such modifications, preferably 2 or 3 such modifications) relative to a product of nature, such as a human antibody or a rabbit antibody (such as a polyclonal rabbit antibody) or a murine or rat antibody. In another of such embodiments, the antigen binding construct may be first generated following non-natural immunization of a (species of) mammal; such as by immunization with an antigen to which such (species of) mammal is not exposed in nature, and hence will not have naturally raised antibodies against.

Another aspect of the invention relates to a monoclonal antibody, or a binding fragment thereof, binding to and preferably inhibiting CCR9. The present invention describes CCR9 as a target for modulating immune resistance of a tumor disease. Therefore, the present invention relates to the use of CCR9 as a novel target for the generation of modulating, such as inhibitory, antibodies directed against the CCR9 protein. The generation of such antibodies is as such a standard procedure for the skilled artisan, and the modulating activity in respect of CCR9 may be investigated by one or more of the methods disclosed elsewhere herein, such as in the examples.

The anti-CCR9 antibodies of the invention may be monoclonal or polyclonal antibodies. Monoclonal antibodies may be prepared using hybridoma-based methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

An immunizing agent typically includes the CCR9, protein, or fragments thereof, or a fusion protein thereof. However, antibodies may be prepared by genetic immunization methods in which native proteins are expressed in vivo with normal post-transcriptional modifications, avoiding antigen isolation or synthesis. For example, hydrodynamic tail or limb vein delivery of naked plasmid DNA expression vectors (eg, those encoding protein of CCR9) can be used to produce the antigen of interest in vivo in mice, rats, and rabbits and thereby induce antigenspecific antibodies (Tang et al, Nature 356(6365): 152-4 (1992); Tighe et al, Immunol. Today 19(2) 89-97 (1998); Bates et al, Biotechniques, 40(2) 199-208 (2006); Aldevron-Genovac, Freiburg DE). This allows the efficient generation of high-titre, antigen-specific antibodies which may be particularly useful for diagnostic and/or research purposes. A variety of gene delivery methods can be used, including direct injection of naked plasmid DNA into skeletal muscle, lymph nodes, or the dermis, electroporation, ballistic (gene gun) delivery, and viral vector delivery.

Generally, either peripheral blood lymphocytes (“PBLs”) from the immunized host animal are isolated and used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding (1986) Monoclonal Antibodies: Principles and Practice, Academic Press, pp. 59-103). Immortalized cell lines may be transformed mammalian cells, particularly myeloma cells of rodent, bovine, and human origin. Rat- or mouse-myeloma cell lines may be employed. The hybridoma cells may be cultured in a suitable culture medium that preferably contains one or more substances that inhibit the growth or survival of the unfused, immortalized cells. For example, if the parental cells lack the enzyme hypoxanthine guaninphosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor (1984) J. Immunol. 133:3001; Brodeur et al (1987) Monoclonal Antibody Production Techniques and Applications, Marcel Dekker, Inc., New York, pp. 51-631).

The culture medium in which the hybridoma cells are cultured can then be assayed for the presence of monoclonal antibodies directed against CCR9 protein. The binding specificity of monoclonal antibodies produced by the hybridoma cells can be determined by inmunoprecipitation or by an in vitro binding assay, such as radioimmunoassay (RIA) or enzyme-linked immunosorbent assay (ELISA). Such techniques and assays are known in the art. The binding affinity of the monoclonal antibody can be determined, for example, by the Scatchard analysis of Munson and Pollard (1980) Anal. Biochem. 107:220. Furthermore, in order to identify antibodies that inhibit the expression, function and/or stability of CCR9, the candidate antibodies can be used in the herein described TIL screening setup (see example section), or the herein described screening method of the invention. In particular, such antibodies are selected which increase the tumor cell susceptibility to TILs.

After the desired hybridoma cells are identified, the clones may be subcloned by limiting dilution procedures and grown by standard methods. The monoclonal antibodies secreted by the subclones may be isolated or purified from the culture medium or ascites fluid by conventional immunoglobulin purification procedures, such as, protein A-Sepharose, hydroxylapatite chromatography, gel electrophoresis, dialysis, or affinity chromatography.

Monoclonal antibodies of the present invention may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. The DNA also may be modified, for example, by substituting the coding sequence for human heavy and light chain constant domains in place of the homologous murine sequences (U.S. Pat. No. 4,816,567; Morrison et al., Proc Natl Acad Sci USA. 1984 November; 81(21): 6851-6855) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

An antibody of the present invention may be a mouse, rat, rabbit, horse, goat, antibody, or a humanized or chimeric antibody. Most preferably, the antibody of the invention has an inhibitory effect, on the immune modulatory function of CCR9 as described in context of the herein disclosed invention.

Antisense Molecules and Other Nucleic Acid Inhibitors

As described above, inhibitors or antagonists of the negative regulators the subject matter of the present invention (eg CCR9) are in another embodiment, preferably, a nucleic acid molecule, such as an inhibitory nucleic acid molecule eg an antisense molecule.

An inhibitor of eg CCR9 expression or an inhibitor of eg CCR9-T-cell interaction that is a nucleic acid can be, for example, an anti-sense nucleotide sequence, an RNA molecule, or an aptamer sequence. An anti-sense nucleotide sequence can bind to a nucleotide sequence within a cell and modulate the level of expression of CCR9 or modulate expression of another gene that controls the expression or activity of CCR9. Similarly, an RNA molecule, such as a catalytic ribozyme, can bind to and alter the expression of the CCR9 gene, or other gene that controls the expression or activity of CCR9. An aptamer is a nucleic acid sequence that has a three dimensional structure capable of binding to a molecular target.

Certain preferred embodiments pertain to genetic constructs for gene editing that are used as inhibitors of CCR9 in context of the herein described invention. By using gene editing it is possible to modulate the expression, stability or activity of CCR9. Gene editing approaches are well known in the art and may be easily applied when the target gene sequences are known. Preferably such approaches may be used in gene therapy using e.g. viral vectors which specifically target tumor cells in accordance with the above descriptions. Gene editing involves the use of a gene editing DNA endonuclease enzyme (e.g. CRISPR/Cas9) in combination with a guide RNA or guide DNA (gRNA/gDNA) which binds to the gene editing DNA endonuclease enzyme and directs the enzyme to the targeted site in the genome by sequence complementarity of the gRNA/gDNA. A detailed summary of gene editing and its therapeutic approach is provided for example in: Savid N and Schwank G, Transl Res. 2016 February; 168:15-21. doi: 10.1016/j.trsl.2015.09.008. Review. PubMed PMID:26470680.

In certain embodiments, the inhibitor of CCR9 expression or inhibitor of CCR9-T cell interaction or inhibitor of CCR9 signalling of the invention is a targeted gene editing construct, such as a CRISPR/Cas9 construct and/or guide RNA/DNA (gRNA/gDNA).

An inhibitor of CCR9 expression or an inhibitor of CCR9-T-cell interaction that is a nucleic acid also can be a double-stranded RNA molecule for use in RNA interference methods. RNA interference (RNAi) is a process of sequence-specific gene silencing by post-transcriptional RNA degradation or silencing. The RNAi is initiated by use of double-stranded RNA (dsRNA) that is homologous in sequence to the target gene to be silenced. A suitable doublestranded RNA (dsRNA) for RNAi contains sense and antisense strands of about 21 contiguous nucleotides corresponding to the gene to be targeted that form 19 RNA base pairs, leaving overhangs of two nucleotides at each 3′ end (Elbashir et al., Nature 411:494-498 (2001); Bass, Nature 411:428-429 (2001); Zamore, Nat. Struct. Biol. 8:746-750 (2001)). dsRNAs of about 25-30 nucleotides have also been used successfully for RNAi (Karabinos et al., Proc. Natl. Acad. Sci. USA 98:7863-7868 (2001). dsRNA can be synthesized in vitro and introduced into a cell by methods known in the art.

A particularly preferred example of an antisense molecule of the invention is a small interfering RNA (siRNA) or endoribonuclease-prepared siRNA (esiRNA). An esiRNA is a mixture of siRNA oligos resulting from cleavage of a long double-stranded RNA (dsRNA) with an endoribonuclease such as Escherichia coli RNase III or dicer. esiRNAs are an alternative concept to the usage of chemically synthesized siRNA for RNA Interference (RNAi). An esiRNAs is the enzymatic digestion of a long double stranded RNA in vitro.

As described above, a modulator of the invention that is an RNAi molecule (such as an siRNA) may bind to and directly inhibit or antagonise the expression of mRNA of CCR9. However, a modulator of the invention that is an RNAi molecule (such as an siRNA) may bind to and inhibit or antagonise the expression of mRNA of another gene that itself controls the expression (or function or stability) of CCR9. Such other genes may include transcription factors or repressor proteins.

The sequence identity of the antisense molecule according to the invention in order to target a CCR9 mRNA (or to target mRNA of a gene controlling expression, function and/or stability CCR9), is with increasing preference at least 75%, at least 80%, at least 85%, at least 90%, at least 95%, at least 98%, at least 99% and 100% identity to a region of a sequence encoding the CCR9 protein, (eg the amino acid sequence SEQ ID NO. 1 or 2) such as that nucleic acid sequence of CCR9 as disclosed herein (SEQ ID NO. 3 or 4) (or of such other controlling gene). Preferably, the region of sequence identity between the target gene and the modulating antisense molecule is the region of the target gene corresponding to the location and length of the modulating antisense molecule. For example, such a sequence identity over a region of about 19 to 21 bp of length corresponding to the modulating siRNA or shRNA molecule). Means and methods for determining sequence identity are known in the art. Preferably, the BLAST (Basic Local Alignment Search Tool) program is used for determining the sequence identity with regard to one or more CCR9 RNAs as known in the art. On the other hand, preferred antisense molecules such as siRNAs and shRNAs of the present invention are preferably chemically synthesized using appropriately protected ribonucleoside phosphoramidites and a conventional RNA synthesizer. Suppliers of RNA synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

The ability of antisense molecules, siRNA, and shRNA to potently, but reversibly, silence genes in vivo makes these molecules particularly well suited for use in the pharmaceutical composition of the invention which will be also described herein below. Ways of administering siRNA to humans are described in De Fougerolles et al., Current Opinion in Pharmacology, 2008, 8:280-285. Such ways are also suitable for administering other small RNA molecules like shRNA. Accordingly, such pharmaceutical compositions may be administered directly formulated as a saline, via liposome based and polymer-based nanoparticle approaches, as conjugated or complexation pharmaceutical compositions, or via viral delivery systems. Direct administration comprises injection into tissue, intranasal and intratracheal administration. Liposome based and polymer-based nanoparticle approaches comprise the cationic lipid Genzyme Lipid (GL) 67, cationic liposomes, chitosan nanoparticles and cationic cell penetrating peptides (CPPs). Conjugated or complexation pharmaceutical compositions comprise PEIcomplexed antisense molecules, siRNA, shRNA or miRNA. Further, viral delivery systems comprise influenza virus envelopes and virosomes.

The antisense molecules, siRNAs, shRNAs may comprise modified nucleotides such as locked nucleic acids (LNAs). The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers are synthesized chemically and are commercially available. The locked ribose conformation enhances base stacking and backbone pre-organization. This significantly increases the hybridization properties (melting temperature) of oligonucleotides. Particularly preferred example of siRNAs is GapmeR (LNA™ GapmeRs (Exiqon)). GapmeRs are potent antisense oligonucleotides used for highly efficient inhibition of CCR9 mRNA (or of mRNA of a gene controlling expression, function and/or stability of CCR9). GapmeRs contain a central stretch of DNA monomers flanked by blocks of LNAs. The GapmeRs are preferably 14-16 nucleotides in length and are optionally fully phosphorothioated. The DNA gap activates the RNAse H-mediated degradation of targeted RNAs and is also suitable to target transcripts directly in the nucleus.

Preferred antisense molecules for targeting CCR9, are antisense molecules or constructs having a sequence complementary to a region (such as one described above) of a nucleic acid sequence of a CCR9 mRNA, preferably a sequence complementary to a region of a sequence encoding the amino acid sequence of CCR9 shown in SEQ ID NO. 1 or 2 (such as, a sequence complementary to a region of the nucleic acid sequence of CCR9 shown in SEQ ID NO 3 or 4), more preferably, a sequence complementary to a region of between about 15 to 25 bp (such as between about 19 and 21 bp) of a sequence encoding the amino acid sequence shown in SEQ ID NO. 1 or 2 (such as, a sequence complementary to such a region of the nucleic acid sequence of CCR9 shown in SEQ ID NO 3 or 4). Most preferred is an antisense molecule comprising, or consisting essentially of, a sequence according to an shRNA having a sequence at least 90% identical to a sequence according to SEQ ID NO. 5. In another preferred embodiments, the modulating shRNA molecule comprises, or consists essentially of, a sequence identical to a sequence according to SEQ ID NO. 5, optionally with no more than five, four, three, two or one, most preferably no more than two or one, nucleotide substitution or deletion compared to such sequence.

In one embodiment the antisense molecules of the invention may be isolated. In another embodiment, the antisense molecules of the invention may be recombinant, synthetic and/or modified, or in any other way non-natural or not a product of nature. For example, a nucleic acid of the invention may contain at least one nucleic acid substitution (or deletion) modification such as between 1 and about 5 such modifications, preferably no more than 1, 2 or 3 such modifications) relative to a product of nature, such as a human nucleic acid. As described above, the antisense molecules of the invention may be modified by use of non-natural nucleotides, or may be conjugated to another chemical moiety. For example, such chemical moieties may be a heterologous nucleic acid conferring increased stability or cell/nucleus penetration or targeting, or may be a non-nucleic acid chemical moiety conferring such properties, of may be a label.

Further Methods of Treatment Related to Gene-mediated Immune Resistance

An embodiment of a method of treatment of the invention preferably, comprises a step of contacting the tumor cell with an inhibitor of the expression of a negative regulator the subject matter of the present invention (such as CCR9), an inhibitor of such gene's signalling or an inhibitor of CCR9-T-cell interaction.

In context of the invention it was surprisingly found that CCR9 and the other modulator genes mediate tumor resistance against cytotoxic T lymphocytes (CTL) by direct contact of the tumor cell and the CTL. Therefore, the present invention for the first time indicates a method for reducing tumor resistance to CTL responses by impairing the eg CCR9 mediated interaction between the tumor cell and the CTL.

Thus, in certain preferred embodiments said tumor cell is characterized by a detectable expression of CCR9 protein or mRNA, such as cell surface expression of CCR9 (protein) before contacting the tumor cell with an inhibitor of CCR9 expression or an inhibitor of CCR9-T-cell interaction or an inhibitor of CCR9 signalling.

In another aspect, the invention provides a method of treating a tumor disease in a patient, wherein said tumor disease is characterized by a resistance of said tumor against autologous T-cell mediated immune responses, the method comprising a step of inhibiting in said patient CCR9 expression in said tumor, and/or inhibiting in said patient CCR9 mediated interaction of at least one tumor cell of said tumor with at least one T-cell of said patient and/or inhibiting in said patient CCR9 signalling in said tumor.

Some embodiments of the invention pertain to a method wherein the inhibitor of CCR9-T-cell interaction is an inhibitor of CCR9 mediated STAT1 impairment of T-cells.

Another aspect of the invention pertains to a method for aiding a patient's immune response against a tumor disease comprising a step of inhibiting in said patient CCR9 expression in said tumor, and/or inhibiting in said patient CCR9 mediated interaction of at least one tumor cell of said tumor with at least one T-cell of said patient and/or inhibiting in said patient CCR9 signalling in said tumor.

Certain embodiments of these methods may comprise a step of administering to said patient a therapeutically effective amount of an inhibitor of CCR9 expression and/or an inhibitor of CCR9-T-cell interaction and/or an inhibitor of CCR9 signalling, as described herein before.

Particularly preferred inhibitors of CCR9 expression or inhibitors of CCR9-T-cell interaction or inhibitors of CCR9 signalling are compounds selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct and/or guide RNA/DNA (gRNA/gDNA), a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds. Most preferred are CCR9 inhibitors or antagonists that are antigen binding constructs, such as an antibodies (or derivatives thereof), or nucleic acid molecules, such as inhibitory nucleic acid molecules. Such most preferred embodiments of CCR9 inhibitors or antagonists are described in more detail elsewhere herein.

In some particular aspects of the invention, it may be preferably that the inhibitor of CCR9-T-cell interaction or inhibitor of CCR9 signalling is selectively inhibiting the function of CCR9 as a tumor resistance factor against CTL responses, and not of CCR9 mediated chemotaxis.

A tumor or tumor disease of the invention may be selected from a liquid or solid tumor, and preferably is breast cancer, ovarian cancer, cancer of the colon and generally the gastrointestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia. A tumor cell, in the context of the present invention, may be a cell of, from or derived from any of such tumor or tumor diseases.

In the context of the present invention, the CCR9-T-cell interaction is preferably mediated by CCR9, such as an interaction of CCR9 with a T-cell, for example by intermolecular interaction between cell surface expressed CCR9 on said tumor cell and at least one T-cell component expressed on the cellular surface of said T-cell.

Some aspects of the invention also pertain to an inhibitor or antagonist of (a) (eg of CCR9) and/or an activator or agonist of (b), in each case as described above for use in a method as described herein above.

Yet a further aspect of the invention provides a method for identifying a (therapeutic) compound suitable for the treatment of a tumor disease. In one embodiment of such aspects, the method comprising the steps of
(a) Providing a first cell expressing a protein (or mRNA) of CCR9, preferably expressing CCR9 protein on the cellular surface,
(b) Providing a candidate compound,
(c) Optionally, providing a second cell which is a cytotoxic T-lymphocyte (CTL), preferably that is capable of immunologically recognizing said first cell, and (d) Bringing into contact the first cell and the candidate compound, and optionally the second cell, and
(e) Determining subsequent to step (d), either or both of:
   i. expression of said protein (or mRNA) of CCR9 in said first cell, wherein a reduced expression of said protein (or mRNA) of CCR9 in said first cell contacted with the candidate compound compared to said first cell not contacted with said candidate compound indicates that the candidate compound is a compound suitable for the treatment of a tumor disease; and/or
   ii. cytotoxicity of said CTL against said first cell, wherein an enhanced cytotoxicity of said CTL against said first cell contacted with the candidate compound compared to the cytotoxicity of said CTL against said first cell not contacted with the candidate compound indicates that the candidate compound is a compound suitable for the treatment of a tumor disease.

In another of such aspects the method comprising the steps of
(a*) Providing a first cell expressing a CCR9 protein on the cellular surface,
(b*) Contacting said first cell with a candidate compound,
(c*) And/or, contacting subsequent to step (b*) said first cell with a cytotoxic T-lymphocyte (CTL), and
(d*) Determining subsequent to step (b*) and/or (c*) CCR9 expression in said first cell, wherein a reduced CCR9 expression in said first cell contacted with the candidate compound compared to said first cell not contacted with said candidate compound indicates that the candidate compound is a (therapeutic) compound suitable for the treatment of a tumor disease; and/or
(e*) Determining subsequent to step (c*) cytotoxicity of said CTL against said first cell, wherein an enhanced cytotoxicity of said CTL against said first cell contacted with the candidate compound compared to the cytotoxicity of said CTL against said first cell not contacted with the candidate compound indicates that the candidate compound is a (therapeutic) compound suitable for the treatment of a tumor disease.

As described above, CCR9 is described herein as one specifically illustrated embodiment of the various other embodiments that may equally apply for each aspect of the invention—such as such screening methods—in respect of the described inhibitors and antagonists of proteins (or mRNAs) of the other (negative regulator) genes, or correspondingly in respect of the described activators and agonists of proteins (or mRNAs) of the (positive regulator) genes, which genes have in each case been identified as being modulators of tumor resistance against immune response, and hence are the subject matter of the present invention.

Accordingly, in another embodiment of such screening aspects, the method comprising the steps of
(a) Providing a first cell expressing a protein on the cellular surface, wherein the protein is selected from: (x) FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9 (or (i) OR2J2, VN1R4 or OR1F1; or (ii) CEACAM-6 or CD272), or is selected from: (y) ENPP2, GRM6, GRK5, OR1G1, CCR2, ADMR, GRM4, MASS1, CXCL9, CXCR3, or OR1D4,
(b) Providing a candidate compound,
(c) Optionally, providing a second cell which is a cytotoxic T-lymphocyte (CTL), preferably that is capable of immunologically recognizing said first cell, and
(d) Bringing into contact the first cell and the candidate compound and optionally the second cell, and
(e) Determining subsequent to step (d), either or both of
   i. expression/function of said protein in said first cell, wherein a differential protein expression in said first cell contacted with the candidate compound compared to said first cell not contacted with said candidate compound indicates that the candidate compound is a compound suitable for the treatment of a tumor disease; and/or
   ii. cytotoxicity of said CTL against said first cell, wherein an enhanced cytotoxicity of said CTL against said first cell contacted with the candidate compound compared to the cytotoxicity of said CTL against said first cell not contacted with the candidate compound indicates that the candidate compound is a compound suitable for the treatment of a tumor disease.

And, in another embodiment of such screening aspects, the method comprising the steps of
(a*) Providing a first cell expressing a protein on the cellular surface, wherein the protein is selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, or is selected from ENPP2, GRM6, GRK5, OR1G1, CCR2, ADMR, GRM4, MASS1, CXCL9, CXCR3, or OR1D4,
(b*) Contacting said first cell with a candidate compound,
(c*) And/or, contacting subsequent to step (b*) said first cell with a cytotoxic T-lymphocyte (CTL), and
(d*) Determining subsequent to step (b*) and/or (c*) protein expression/function in said first cell, wherein a differential protein expression in said first cell contacted with the candidate compound compared to said first cell not contacted with said candidate compound indicates that the candidate compound is a (therapeutic) compound suitable for the treatment of a tumor disease; and/or
(e*) Determining subsequent to step (c*) cytotoxicity of said CTL against said first cell, wherein an enhanced cytotoxicity of said CTL against said first cell contacted with the candidate compound compared to the cytotoxicity of said CTL against said first cell not contacted with the candidate compound indicates that the candidate compound is a (therapeutic) compound suitable for the treatment of a tumor disease.

In some embodiments, a reduced protein expression/function of a protein selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3 (or (i) OR2J2, VN1R4 or OR1F1; or (ii) CEACAM-6 or CD272) in said first cell contacted with the candidate compound compared to said first cell not contacted with said candidate compound indicates that the candidate compound is a therapeutic compound suitable for the treatment of a tumor disease.

In alternative embodiments, an increased protein expression/function of a protein selected from ENPP2, GRM6, GRK5, OR1G1, CCR2, ADMR, GRM4, MASS1, CXCL9, CXCR3, or OR1D4 in said first cell contacted with the candidate compound compared to said first cell not contacted with said candidate compound indicates that the candidate compound is a therapeutic compound suitable for the treatment of a tumor disease.

In some embodiments of such screening methods said first cell is a cell resistant to cytotoxicity mediated by T-lymphocytes, preferably a tumor derived cell.

The tumor disease in such methods may, in particular embodiments, be selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia. Accordingly, the tumor derived cell in such methods may, in particular embodiments, be a cell or of derived from any of such tumor diseases.

The tumor disease may be one characterized by a resistance against T cell mediated immune responses.

For the screening methods of the invention a candidate compound may be selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct and/or guide RNA/DNA (gRNA/gDNA), a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

Another aspect of the invention further pertains to a method for diagnosing, in a patient, a resistance of a tumor disease against T cell mediated immune responses. The diagnostic method comprises a step of determining expression of CCR9 in a tumor cell from the tumor of the patient, wherein a detectable expression of CCR9 in the tumor cell compared to a negative control is indicative for a resistance of the tumor disease against T cell mediated immune responses. The expression of CCR9 may be determined by detection of the present (or an amount of) CCR9 mRNA and/or CCR9 protein, such as CCR9 protein expressed on the surface of the tumor cell.

NEW from here: As described above, CCR9 is described herein as one specifically illustrated embodiment of the various other embodiments that may equally apply for each aspect of the invention—such as such diagnostic method—in respect of the described inhibitors and antagonists of proteins (or mRNAs) of the other (negative regulator) genes, or correspondingly in respect of the described activators and agonists of proteins (or mRNAs) of the (positive regulator) genes, which genes have in each case been identified as being modulators of tumor resistance against immune response, and hence are the subject matter of the present invention.

Accordingly, another embodiment of such diagnostic aspect comprises a method for diagnosing in a patient a resistance of a tumor disease against T cell mediated immune responses, the method comprising a step of
(a) determining expression of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9 (or (i) OR2J2, VN1R4 or OR1F1; or (ii) CEACAM-6 or CD272) in a tumor cell from the tumor of the patient, wherein a detectable or increased expression of any one of the proteins in the tumor cell compared to a negative control is indicative for a resistance of the tumor disease against T cell mediated immune responses; or
(b) determining expression of: ENPP2, GRM6, GRK5, OR1G1, CCR2, ADMR, GRM4, MASS1, CXCL9, CXCR3, or OR1D4 in a tumor cell from the tumor of the patient, wherein a reduced expression of any one of the proteins in the tumor cell compared to a negative control is indicative for a resistance of the tumor disease against T cell mediated immune responses.

In certain embodiments, the expression of a gene of (a) or (b) above and/or its cognitive ligand or receptor (as applicable) is determined. Examples of corresponding ligand/receptor pairs include IL8/ILRA8, CCL2/CCR2 and CXCL9/CXCR3 The expression of the respective gene may be determined by detection of the present (or an amount of) such gene's mRNA and/or protein, such as such gene's protein expressed on the surface of the tumor cell.

The diagnostic method may comprise a preceding step of obtaining a tumor cell from the patient.

The diagnostic method may comprise a step of determining the resistance of tumor cells (such as obtained from the patient) against a T cell mediated immune response. Such an embodiment may further include contacting said tumor cells with (eg HLA-matched) cytotoxic T cells and determining the degree of lysis of said tumor cells, for example: (i) relative to one or more controls such as tumor cells having reduced CCR9 expression or function (eg mediated by an inhibitory anti-CCR9 antibody and/or a anti-CCR9 siRNA) and/or in the absence of cytotoxic T cells; or (ii) relative to one or more controls such as tumor cells having reduced expression or function of the respective gene (eg mediated by an inhibitory antibody and/or an siRNA against such gene) and/or in the absence of cytotoxic T cells

In certain embodiments of such diagnostic methods, said tumor disease is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia. Accordingly, the tumor cell, if obtained from the patient, may be a cell of or derived from any of such tumor diseases.

In some preferred embodiments, the diagnostic method of the invention is an in-vitro or ex-vivo method.

The present invention also provides a kit (such as a detection and/or diagnostic kit) comprising means for the determination of the presence or absence of the respective gene, such as in or on the surface of a cell associated with a tumor or tumor disease. The diagnostic kit is suitable for detecting or diagnosing an absent or decreased immune susceptibility of a tumor, tumor disease or tumor cell to an immune response, such as towards a cell-mediated immune response (eg, for detecting or diagnosing a resistance of a tumor disease against T cell mediated immune responses). The kit may preferably comprise specific and selective antibodies against the respective gene as described herein before. Alternatively, the diagnostic kit may comprise nucleic acid primers and/or probes for detecting the expression of the respective gene in a tumor cell. The kit of the invention may include other known means for detecting the respective gene protein or mRNA expression.

The kit of the invention may further comprise instructions for use and/or with one or more additional components useful for said detection. Such instructions may consist of a printed manual or computer readable memory comprising such instructions, or may comprise instructions as to identify, obtain and/or use one or more other components to be used together with the kit. Such additional component may comprise one or more other item, component, reagent or other means useful for the use of the kit or practice of a detection method of the invention, including any such item, component, reagent or means disclosed herein useful for such practice. For example, the kit may further comprise reaction and/or binding buffers, labels, enzymatic substrates, secondary antibodies and control samples, materials or moieties etc.

In preferred embodiments of the kit or the detection/diagnostic methods, the means for the detection of protein or mRNA of the respective gene is labelled; for example is coupled to a detectable label. The term “label” or “labelling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, 3-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.).

The present invention in one additional aspect solves the problems in the prior art by providing a combination comprising (a) and, (b) and/or (c), wherein
(a) is an inhibitor or antagonist of CCR9,
(b) is an inhibitor or antagonist of PI3K-Akt signaling and/or an inhibitor or antagonist of p70S6 kinase signaling, and
(c) is an activator or agonist of ERK1/2 signalling and/or and activator or inhibitor of JNK signalling.

In context of the present invention is was furthermore surprisingly found that the CCR9 mediated resistance of tumor cells against CTL responses resulted in elevated signalling in (such as, but without being bound by theory, is signalled via) PI3K-Akt signaling and p70S6 kinase signaling, i.e. which is associated with activated CCR9 function (eg, such signalling is activated by (or may activate) CCR9 function). In contrast, it was found that the CCR9 mediated resistance of tumor cells against CTL responses resulted in reduced ERK1/2 signaling and JNK signaling, i.e. which is associated with antagonized CCR9 function (eg, such signalling is antagonized by (or may antagonize) CCR9 function as a CTL inhibitor. In this aspect, the previously mentioned in context of the inhibitor or antagonist of CCR9 or the treatment of the tumor disease, and the kind of tumor diseases equally applies in this aspect. The combination as described herein is a further invention developed based on the findings mentioned above.

As described above, CCR9 is described herein as one specifically illustrated embodiment of the various other embodiments that may equally apply for each aspect of the invention—such as such combinations—in respect of the described inhibitors and antagonists of proteins (or mRNAs) of the other (negative regulator) genes, or correspondingly in respect of the described activators and agonists of proteins (or mRNAs) of the (positive regulator) genes, which genes have in each case been identified as being modulators of tumor resistance against immune response, and hence are the subject matter of the present invention.

Accordingly, another embodiment of such combination aspect comprises a combination comprising (a) and (b) or (a) and (c) or (a), (b) and (c), wherein
(a) Is an inhibitor or antagonist of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9,
(b) Is an inhibitor or antagonist of PI3K-Akt signaling and/or an inhibitor or antagonist of p70S6 kinase signaling, and
(c) Is an activator or agonist of ERK1/2 signaling and/or an activator or agonist of JNK signaling.

In other embodiments, inhibitor or antagonist of (a) above is an inhibitor or antagonist of the respective gene and/or its corresponding ligand or receptor (as applicable).

Accordingly further, another embodiment of such combination aspect comprises a combination comprising (a) and (b) or (a) and (c) or (a), (b) and (c), wherein
(a) Is an inhibitor or antagonist of: OR2J2, VN1R4 or OR1F1,
(b) Is an inhibitor or antagonist of PI3K-Akt signaling and/or an inhibitor or antagonist of p70S6 kinase signaling, and
(c) Is an activator or agonist of ERK1/2 signaling and/or an activator or agonist of JNK signaling.

Accordingly further, another embodiment of such combination aspect comprises a combination comprising (a) and (b) or (a) and (c) or (a), (b) and (c), wherein
(a) Is an inhibitor or antagonist of: CEACAM-6 or CD274,
(b) Is an inhibitor or antagonist of PI3K-Akt signaling and/or an inhibitor or antagonist of p70S6 kinase signaling, and
(c) Is an activator or agonist of ERK1/2 signaling and/or an activator or agonist of JNK signaling.

In one preferred of said embodiments, said inhibitor or antagonist of (a) is an inhibitor or antagonist of FZD3.

In an alternative preferred of said embodiments, said inhibitor or antagonist of (a) is an inhibitor or antagonist of TRHDE.

In an alternative preferred of said embodiments, said inhibitor or antagonist of (a) is an inhibitor or antagonist of IL8.

In an alternative preferred of said embodiments, said inhibitor or antagonist of (a) is an inhibitor or antagonist of OR3A2.

In an alternative preferred of said embodiments, said inhibitor or antagonist of (a) is an inhibitor or antagonist of P2RY11.

In an alternative preferred of said embodiments, said inhibitor or antagonist of (a) is an inhibitor or antagonist of is CCR9.

In an alternative preferred of said embodiments, said inhibitor or antagonist of (a) is an inhibitor or antagonist of OR2J2.

In an alternative preferred of said embodiments, said inhibitor or antagonist of (a) is an inhibitor or antagonist of CEACAM-6.

Accordingly further, another embodiment of such combination aspect comprises a combination comprising (a) and (b) or (a) and (c) or (a), (b) and (c), wherein
(a) Is an activator or agonist of: ENPP2, GRM6, GRK5, OR1G1, CCR2, ADMR, GRM4, MASS1, CXCL9, CXCR3, or OR1D4,
(b) Is an inhibitor or antagonist of PI3K-Akt signaling and/or an inhibitor or antagonist of p70S6 kinase signaling, and
(c) Is an activator or agonist of ERK1/2 signaling and/or an activator or agonist of JNK signaling.

In a preferred of said embodiments, said activator or agonist inhibitor or antagonist of (a) is an activator or agonist inhibitor or antagonist of: one or other member of the receptor/ligand pair CXCL9 or CXCR3.

In another particular preferred of such embodiment, said activator or agonist of (b) is an activator or agonist of GRM4.

In another particular preferred of such embodiment, said activator or agonist of (b) is an activator or agonist of GRK5.

In another particular preferred of such embodiment, said activator or agonist of (b) is an activator or agonist of CCR2.

In other aspects/embodiments, the inhibitor or antagonist of a gene of (a) above and/or its cognitive ligand or receptor (as applicable). In alternative embodiments, the modulator of tumor resistance is an activator or agonist of a gene of (b) above and/or its corresponding ligand or receptor (as applicable). Table 2 lists the corresponding receptor/ligand pairs for the negative and positive regulators of tumor cell resistance the subject matter of the present invention.

In one preferred embodiment, the combination comprises (a) and, (b) and/or (c), wherein
(a) is an inhibitor/antagonist or activator/agonist of any (a) described above,
(b) is an inhibitor or antagonist of PI3K-Akt signaling or an inhibitor or antagonist of p70S6 kinase signaling, and
(c) is an activator or agonist of ERK1/2 signalling.

In a second preferred embodiment, the combination comprises (a) and (b), wherein
(a) is an inhibitor/antagonist or activator/agonist of any (a) described above, and
(b) is an inhibitor or antagonist of PI3K-Akt signalling.

In a third preferred embodiment, the combination comprises (a) and (b), wherein
(a) is an inhibitor/antagonist or activator/agonist of any (a) described above, and
(b) is an inhibitor or antagonist of p70S6 kinase signalling.

In a fourth preferred embodiment, the combination comprises (a) and (c), wherein
(a) is an inhibitor/antagonist or activator/agonist of any (a) described above, and
(c) is an activator or agonist of ERK1/2 signalling.

In a fifth preferred embodiment, the combination comprises (a) and (c), wherein
(a) is an inhibitor/antagonist or activator/agonist of any (a) described above, and
(c) is an activator or agonist of JNK signalling.

In certain of each preferred embodiments of such combinations, component (a) of the combination is an antigen binding construct that binds (preferably specifically) the protein of the respective gene, such as an antigen binding contract described above. For example, component (a) of the combination may be an antibody that binds to (protein of) the respective gene and inhibits expression of the respective gene and/or interaction between protein of of the respective gene and a T-cell and/or cell signalling mediated by the respective gene.

In other certain of each preferred embodiments of such combinations, component (a) of the combination is a nucleic acid, such as an inhibitory nucleic acid molecule. For example, such a nucleic acid may be an antisense molecule or an siRNA, that inhibits expression of the respective gene and/or interaction between protein of of the respective gene and a T-cell and/or cell signalling mediated by the respective gene.

The combinations of the invention are preferably for use in medicine; in particular embodiments thereof for use in the treatment of a tumor disease of a subject, such as a tumor disease that is characterized by resistance to such immune response and/or is characterised by expression of the respective gene. Exemplary such tumor diseases are described elsewhere herein.

The term “combination”, when used in this context, is intended to mean any physical or methodological combination of the individual components that is suitable for such medical use. By way of non-limiting examples, a combination of the invention may be described by the following:

Co-Formulation:

A mixture comprising two or more of the respective components (a) and, (b) and/or (c) (such as in any of the specific preferred combinations disclosed above), preferably formulated with one or more pharmaceutically acceptable carriers, suitable for administration to a subject in need. Such a co-formulated combination of the invention may be provided or administered to the subject in any of pharmaceutical forms (such as those described elsewhere herein) that is suitable for the subject, tumor disease and/or mode or administration. As will be appreciated, administration of a co-formulated combination of the invention will report in essentially concomitant administration to the subject of the individual components of the combination comprised therein. However, although such components may be so administered (essentially) concomitant, the person of ordinary will appreciate that depending on formulation of the individual components (for example delayed release coating) and/or the pharmacokinetic properties of the active ingredients of each component, the exposure of tumor/tumor cell in the subject to a therapeutically effective amount of one or more of the components resulting from such administration may—indeed—be temporally offset to that of the other components(s).

Co-Package:

At least one component of the combination of the invention is formulated, stored, transported and/or packaged separately from the other components. In one embodiment, such a co-packaged combination may consist of a pharmaceutical package may be manufactured that contains separate containers, wherein at least two of such containers comprise different components of the combination. Such a co-package combination may also be described as a “combination kit”. For example, one container in such a package may be a prefilled syringe (or vial) comprising component (a), and a second containers in the package may be another pre-filled syringe (or vial) comprising one or more of the component(s) (b) and/or (c) (such as—together—forming any of the specific preferred combinations disclosed above), in each case optionally formulated with pharmaceutically acceptable carriers. In one embodiment, the individual components of such co-packaged combination embodiment of the combination may be used to prepare a co-formulation (such as described above) for administration to the subject. Such an embodiment may be suitable in those circumstances where (essentially) concomitant administration of two or more components of the combination by the same administration route is desired, but such individual components are not already provided as a co-formulation. For example, the individual components may be manufactured and/or sold by different processes or suppliers, or may not be suitable compatible for co-formulation except when needed (eg, if two components were co-formulated in liquid for an extended period, they—or their excipients—may interact with each other in undesired ways). In another embodiment, the individual components of such co-packaged combination may be used to administer to the subject two or more of such components separately to each other, such as in a co-therapy (as described below).

Co-Therapy:

At least one component of the combination of the invention is administered to the subject together with one or more of the other component(s). In one embodiment of such cotherapy, such components may be administered essentially concomitantly (such as by administration of a co-formulation). In a preferred alternative embodiment of such co-therapy, at least one component of the combination is administered to the subject separately from one or more other components(s) of the combination. For example, component (a) of the combination may be administered to the subject separately from components (b) and/or (c) (such as in any of the specific preferred combinations disclosed above). Such separate administration may, in some embodiments, comprise different routes of administration for the respective components (eg, using two or more suitable routes of administration as described elsewhere herein). Such separate administration may, in some alternative embodiments, may comprise the where the respective components are administered by the same route, but separated by location or time or administration. For example, one component of the combination may be administered by i.m. or i.v. injection into the one arm of a subject, and another component of the combination may be administered by i.m or i.v. injection into another arm of a subject. In another example, one component of the composition may be administered to the subject before or after the administration of another component(s). In this situation, the temporally separated administrations may be made by the same route (eg both oral or both i.v.), or may be made by different routes of administration.

In any of the various embodiments of the combinations, one or more of the components (or the combination as a whole) may be provided together with (for example, the co-packed form of such combination may further include) instructions to administer the combination of the invention to the subject. Such instructions may, for example, describe the route of administration, dosage and/or respective timing of the respective component(s) of the combination, and/or it may describe how to prepare one or more of the components for co-formulation and/or co-therapy.

The various components (a) and, (b) and/or (c) of a combination of the invention (such as in any of the specific preferred combinations disclosed above) will, in preferred embodiments, be used with the subject in a therapeutically effective amount (or dose).

Hence, in one embodiment a combination may be provided as a pharmaceutical composition comprising (a) and, (b) and/or (c) (such as in any of the specific preferred combinations disclosed above). Such combination may be a pharmaceutical composition comprising two or more of such components (such as a co-formulated combination), or such combination may comprise a plurality of pharmaceutical compositions different from each other (such as a co-packaged combination). For example, a combination of the invention may comprise at least two pharmaceutical compositions, a first pharmaceutical composition comprising component (a) and a second pharmaceutical composition comprising component (b) and/or (c).

The medical use of the invention is preferably a use in the treatment of a tumor disease. Preferred is that the combination is used in a method for treatment as described in the previous aspects. For example, said tumor, tumor disease (or tumor cell thereof) may be characterized by expression of protein or mRNA of the respective gene, such as detectable cell surface expression of the respective gene (protein). Such characterization may be conducted by a method of diagnosis as described herein. In another example, said tumor cell, tumor or tumor disease may be characterized by a resistance against T-cell mediated cytotoxicity.

By way of further example, in certain preferred embodiments of such use of combination, the tumor or tumor disease (to be) treated is one selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia. Accordingly, such uses include those where a tumor cell is one of or derived from any of such tumors or tumor diseases.

The combination treatment of the invention preferably comprises a step of administering to said patient a therapeutically effective amount of (i) an inhibitor (or activator, as applicable) of expression of the respective gene and/or an inhibitor (or activator, as applicable) of interaction between a T-cell and protein of the respective gene, (ii) of an inhibitor or antagonist of PI3K-Akt signaling, (iii) of an inhibitor or antagonist of p70S6 kinase signaling, and/or (iv) of an activator or agonist of ERK1/2 signaling or JNK signaling.

In a first related aspect, the invention also provides (i) an inhibitor or antagonist (or activator or agonists, as applicable) of the respective gene, such as any of those describe herein, for use in the treatment of a patient suffering from a tumor or tumor disease (such as one resistant to an immune response, eg a T-cell mediated immune response), by administration of (i) and administration of (ii) an inhibitor or antagonist of PI3K-Akt signalling, and/or (iii) an inhibitor or antagonist of p70S6 kinase signalling, and/or (iv) an activator or agonist of ERK1/2 signalling, and/or (v) an activator or agonist of JNK signalling, for example where administration of (i) and (ii) (and/or (iii) and/or (iv) and/or (v)) occurs within 15 days or each other.

In a second related aspect, the invention also provides (ii) an inhibitor or antagonist of PI3K-Akt signalling, such as any of those describe herein, for use in the treatment of a patient suffering from a tumor or tumor disease (such as one resistant to an immune response, eg a T-cell mediated immune response), by administration of (ii) and administration of (i) an inhibitor or antagonist (or activator or agonists, as applicable) of the respective gene, and optionally said treatment also includes the use of (iii) an inhibitor or antagonist of p70S6 kinase signalling, and/or (iv) an activator or agonist of ERK1/2 signalling, and/or (v) an activator or agonist of JNK signalling, for example where administration of (ii) and (i) (and/optionally (iii) and/or (iv) and/or (v)) occurs within 15 days or each other.

In a third related aspect, the invention also provides (iii) an inhibitor or antagonist of p70S6 kinase signalling, such as any of those describe herein, for use in the treatment of a patient suffering from a tumor or tumor disease (such as one resistant to an immune response, eg a T-cell mediated immune response), by administration of (iii) and administration of (i) an inhibitor or antagonist (or activator or agonists, as applicable) of the respective gene, and optionally said treatment also includes the use of (ii) an inhibitor or antagonist of PI3K-Akt kinase signalling, and/or (iv) an activator or agonist of ERK1/2 signalling, and/or (v) an activator or agonist of JNK signalling, for example where administration of (iii) and (i) (and/optionally (ii) and/or (iv) and/or (v)) occurs within 15 days or each other.

In a fourth related aspect, the invention also provides (iv) an activator or agonist of ERK1/2 signalling, such as any of those describe herein, for use in the treatment of a patient suffering from a tumor or tumor disease (such as one resistant to an immune response, eg a T-cell mediated immune response), by administration of (iv) and administration of (i) an inhibitor or antagonist (or activator or agonists, as applicable) of the respective gene, and optionally said treatment also includes the use of (ii) an inhibitor or antagonist of PI3K-Akt kinase signalling, and/or (iii) an inhibitor or antagonist of p70S6 kinase signalling, and/or (v) an activator or agonist of JNK signalling, for example where administration of (iv) and (i) (and/optionally (ii) and/or (iii) and/or (v)) occurs within 15 days or each other.

In a fifth related aspect, the invention also provides (v) an activator or agonist of JNK signalling, such as any of those describe herein, for use in the treatment of a patient suffering from a tumor or tumor disease, by administration of (v) and administration of (i) an inhibitor or antagonist (or activator or agonists, as applicable) of the respective gene, and optionally said treatment also includes the use of (ii) an inhibitor or antagonist of PI3K-Akt kinase signalling, and/or (iii) an inhibitor or antagonist of p70S6 kinase signalling, and/or (iv) an activator or agonist of ERK1/2 signalling, for example where administration of (v) and (i) (and/optionally (ii) and/or (iii) and/or (iv)) occurs within 15 days or each other.

In a sixth related aspect, the invention also provides a first pharmaceutical composition containing either: (A) an inhibitor or antagonist (or activator or agonists, as applicable) of the respective gene; or (B) an inhibitor or antagonist of PI3K-Akt kinase signalling, and/or an inhibitor or antagonist of p70S6 kinase signalling, and/or an activator or agonist of ERK1/2 signalling and/or an activator or agonist of JNK signalling, wherein said first pharmaceutical composition is for use in the treatment of a patient suffering from a tumor or tumor disease by administration of said first pharmaceutical composition and a second pharmaceutical composition which, in the case of (A) includes the component of (B), or in the case of (B) contains an inhibitor or antagonist (or activator or agonists, as applicable) of the respective gene, for example within 14 days of each other. Preferably, the component of (B) is an inhibitor or antagonist of PI3K-Akt kinase signalling, and/or an inhibitor or antagonist of p70S6 kinase signalling.

In some embodiments of these related aspects, the inhibitor or antagonist of the respective (negative regulator) gene is an inhibitor of expression of such respective gene and/or an inhibitor of interaction between a T-cell and protein of such respective gene and/or an inhibitor of signalling of such respective gene.

In alternative embodiments of these related aspects, the activator or agonist of the respective (positive regulator) gene is an activator of expression of such respective gene and/or an activator of interaction between a T-cell and protein of such respective gene and/or an activator of signalling of such respective gene.

In preferred embodiments of the combination aspects of the invention, said inhibitor (or activator, as applicable) of interaction between a T-cell and protein of the respective gene is an inhibitor (or activator, as applicable) of the respective gene mediated STAT1 impairment in T-cells.

Said inhibitor or antagonist (or activator, as applicable) of component (a) of a composition, said inhibitor or antagonist of PI3K-Akt signaling and/or said inhibitor or antagonist of p70S6 kinase signaling, is a compound selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct and/or guide RNA/DNA (gRNA/gDNA), a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

In a preferred embodiment, the inhibitor or antagonist (or activator or agonist, as applicable) of the respective gene may be an inhibitor or antagonist (or activator or agonist, as applicable) of interaction between a T-cell and protein of the respective gene, in particular those embodiments where said gene-T-cell interaction is a said gene-mediated binding of said tumor cell to said T-cell, for example by intermolecular interaction between cell surface expressed protein of said gene on said tumor cell and at least one T-cell component expressed on the cellular surface of said T-cell.

The combination of the invention may be combined by sequential or concomitant administration to a subject suffering from the tumor disease during said treatment, preferably wherein (a) and (b), or (a) and (c), (b) and (c), or (a) and (b) and (c)) (such as in any of the specific preferred combinations disclosed above) are concomitantly administered during said treatment.

In those embodiments where the (co-therapy) combination comprises sequential administration of the respective components to the subject, then it is preferred that one of such components is administered within about 14 days of another (or the remaining) components of the combination. For example, certain embodiments included where the respective components are administered within 1 day, 2 days, 3 days, 5 days, 7 days, 10 days or 14 days of each other, preferably within 2 days or 1 days of each other. In particular, the respective components are administered within about 48 hours, 24 hours, or 12 hours of each other, or within between about 8 hours, and 4 hours of each other, or between about 2 hours and 30 mins of each other, or within about 15 mins or 5 mins of each other. In alternative embodiments, the administration of the respective components results in the sequential exposure of a cell included in, derived from or being part of the tumor or tumor disease to be treated with active components of the respective components within about 1 day, 2 days, 3 days, 5 days, 7 days, 10 days or 14 days of each other, preferably within 2 days or 1 days of each other. In particular, the respective components are administered so as to result in the sequential exposure of a cell included in, derived from or being part of the tumor or tumor disease to be treated with active components of the respective components within about within about 48 hours, 24 hours, or 12 hours of each other, or within between about 8 hours, and 4 hours of each other, or between about 2 hours and 30 mins of each other, or within about 15 mins or 5 mins of each other.

An “inhibitor or antagonist of PI3K-Akt signaling” or “AKT inhibitor” is any compound that has the effect of preferentially reducing and/or blocking the activity of AKT. The inhibitor may act directly on AKT, for example by preventing phosphorylation of AKT or dephosphorylating AKT, for example at Ser473 and/or Thr308, or alternatively, the inhibitor may act via the inhibition of an upstream activator (or multiple activators) of AKT in the PI3K/AKT/mTOR signalling pathway or other pathway involved in apoptosis, or via the activation of a upstream inhibitor of AKT (for example via mTOR, and/or PI3K and/or PDK1 (aka PDPK1). It is preferred that the AKT inhibitor acts to reduce and/or block the activity of AKT via multiple pathways such that effective inhibition is achieved. Such a compound may, for example, act by inhibition of up-stream effectors/activators of AKT in both the PI3K pathway and the mTOR pathway. Yet further, the inhibitor of AKT may act to prevent or reduce the transcription, translation, post-translational processing and/or mobilisation of AKT (i.e. reduce the expression of AKT), or an upstream activator of the expression of AKT. Alternatively, the “AKT inhibitor” may be a compound that counteracts the survival mechanism modulated by AKT activity by acting downstream of AKT to overcome the action of increased AKT activity. For example, such a compound may induce apoptosis via a mechanism involving AKT but by acting on downstream modulators of AKT, for example, BCL-2 inhibition.

Thus, examples of an “inhibitor or antagonist of PI3K-Akt signaling” or “AKT inhibitor” within the meaning of the present invention include compounds that inhibit PI3K or downstream effectors of PI3K (e.g. PI), compounds that inhibit PDPK1 and/or mTORC2 or associated kinases (e.g. PHT-427 (Meuillet, et al, (2010) Mol Cancer Ther. 9(3): 706-717); BX-795, BX-912 and BX-320 (Chung et al, (2005) Oncogene 24, 7482-7492); and PP-27 and OSI-027 (Evangelisti et al (2011), Leukemia 25, 781-791)), compounds that inhibit AKT directly (i.e. target AKT enzymatic activity) (e.g. AT7867 (Grimshaw K M et al. (2010) Mol Cancer Ther. 9(5):1100-10); KRX-0401 (perifosine) (Kondapaka et al, (2003) Mol Cancer Ther 2: 1093-1103); MK-2206 (Hirai et al. (2010) Mol Cancer Ther 9(7)), compounds that activate PTEN (e.g. Trastuzumab (Nagata et al. (2004) Cancer cell (6))) and any other compounds that lead to a reduction in AKT activation. The compounds may be, for example, small chemical entities, antibodies, small interfering RNA, double-stranded RNA (e.g. RX-0201, A (AKT anti sense)) or Ribozymes. Examples of appropriate small chemical entities include BEZ-235, PI103 (Park et al (2008) Leukemia 22: 1698-1706), API-2, LY294002, Wortmannin, AKT VIII, BKM120, BGT226, Everolimus, Choline kinase inhibitors (e.g. CK37 (Clem et al (2011) Oncogene 1-11); H89 (Wieprecht et al. (1994) Biochem. J. 297, 241-247); MN58b and TCD828 (Tin Chua et al. (2009) Molecular Cancer, 8:131)), bcl-2 inhibitor (e.g. ABT-737), Hsp-90 inhibitors (e.g. Geldanamycin (Stebbins et al (1997) Cell. 89(2): 239-50); and derivatives of Geldanamycin, for example, 17-AAG and 17-DMAG (Hollingshead M et al. (2005) Cancer Chemother Pharmacol. August; 56 (2):115-25), multi-kinase inhibitors (e.g. sunitinib), mTOR kinase inhibitors (e.g. Temsirolimus), proteasome inhibitors (e.g. bortezomib), and TORC1/TORC2 inhibitors (e.g. Palomid 529 (P529)). Further examples of inhibitors of mTOR include rapamycin and rapalogs (rapamycin derivatives) such as deforolimus (AP23573), everolimus (RAD001), and temsirolimus (CCI-779). mTORC1/mTORC2 dual inhibitors (TORCdIs) are designed to compete with ATP in the catalytic site of mTOR. They inhibit all of the kinase-dependent functions of mTORC1 and mTORC2 and therefore, block the feedback activation of PI3K/AKT signaling, unlike rapalogs that only target mTORC1. Compounds with these characteristics such as sapanisertib (codenamed INK128), AZD8055, DS-3078a, OSI-027 and AZD2014 have been developed, and in many cases have entered clinical trials.

Examples of inhibitors of PI3K include: alpelisib (BYL719), BAY-1082439, buparlisib (BKM120), copanlisib (BAY 80-6946), PA-799, pictilisib (GDC-0941), taselisib (GDC-0032), WX-037 and ZSTK-474.

Several, so-called mTOR/PI3K dual inhibitors (TPdIs), have been developed including dactolisib (BEZ-235), BGT226, SF1126, PKI-587, NVPBE235. apitolisib (GDC-0980), gedatolisib (PF-05212384), LY-3023414, omipalisib (GSK2126458), PF-04691502, PKI-179, SF1126 and VS-5584.

Examples of inhibitors of PDK 1/2 include BX-424 (Berlex Biosciences); OSU-03012, OSU03013 (Ohio State University) and compounds described in U.S. Patent Appl. Pub. Nos. 20090209618, 20070286864, the PDK 1/2 inhibitor compounds described therein are incorporated herein by reference.

Further compounds that are inhibitors or antagonists of PI3K-Akt signaling (including those that inhibit AKT directly; i.e. target AKT enzymatic activity) include afuresertib (GSK2110183), ARQ-092 AZD-5363, BAY-1125976, GSK-690693, ipatasertib (GDC-0068 or RG7440), LY-2780301, MK-2206, MSC-2363318A, triciribine (TCN), triciribine phosphate (TCN-P) and uprosertib (GSK2141795 or GSK795). Suitable Akt inhibitors for use in cancer treatment are also disclosed in Nitulescu et al, 2016 (Int J Onc 48:869), the content of which is incorporated by reference herein, specifically Table I and Table II thereof.

A preferred inhibitor or antagonist of PI3K-Akt signaling is one selected from the list consisting of: MK-2206, copanlisib, sapanisertib, alpelisib, buparlisib dactolisib, apitolisib, gedatolisib, omipalisib, afuresertib, ipatasertib, pictilisib, taselisib and uprosertib. In particular embodiments, the inhibitor or antagonist of PI3K-Akt signaling for component (b) of the combination is MK-2206 (also known as M2698; 8-[4-(1-Aminocyclobutyl)phenyl]-9-phenyl-2H-[1,2,4]triazolo[3,4-f][1,6]naphthyridin-3-one; CAS NO: 1032350-13-2), apitolisib, LY-3023414 or copanlisib.

An “inhibitor or antagonist” of P70 S6 kinase siganlling (or simply “SK6 inhibitors”) is any compound that reduces S6K activity, e.g., S6K1 or S6K2 activity. For example, compounds that inhibit S6K enzymatic activity typically bind to an ATP binding site in S6K or bind to a catalytic domain of S6K. The compound preferentially inhibits S6K1 compared to S6K2 or other S6K isoforms, given the difference in phenotypes observed between S6K1 and S6K2 knock out mice. Thus, although compounds that inhibit S6K2 or both S6K1 and S6K2 (such as rapamycin, its derivatives or other mTOR inhibitors) may be useful in context of the present invention. Also included are compounds that reduce SK6 expression, in particular nucleic acid compounds, for example genetic constructs or RNA compounds. Such inhibitors are well known and also described herein above in context of eg CCR9 inhibitors. The similar descriptions apply in the context of SK6 (ie, nucleic acid compounds that reduce S6K expression).

Examples of p70 S6K inhibitors include, and are not limited to compounds described in U.S. Patent Appl. Pub. No. 20080234276, the S6K kinase inhibitors described therein are incorporated herein by reference.

Further compounds that are inhibitors or antagonists of p70 S6 kinase signaling (including those that inhibit S6K directly; i.e. target S6K enzymatic activity) include: LY-2584702, LY2780301 and MSC-2363318A.

A preferred inhibitor or antagonist of p70 S6 kinase signaling is one selected from the list consisting of LY-2584702, LY-2780301 and MSC-2363318A. In particular embodiments, the inhibitor or antagonist of p70 S6 kinase signaling for component (b) of the combination is LY-2780301 or MSC-2363318A.

Indeed, LY-2780301 and MSC-2363318A are dual S6K and Akt inhibitors, and hence are preferred inhibitors of PI3K-Akt signaling and of p70 S6 kinase signaling.

As used herein, the term “activator or agonist of ERK1/2 signaling” means a substance that affects an increase in the amount or rate of ERK1/2 signaling in a cell. Such a substance can act directly, for example, by binding to the ERK kinase and increasing the amount or rate of ERK1/2 signaling component expression or activity. An agonist of ERK1/2 signaling can also increase the amount or rate of ERK expression or activity, for example, by binding to ERK in such a way as to enhance or promote ERK signalling events. An activator or agonist of ERK1/2 signaling can also act indirectly, for example, by binding to a regulatory molecule or gene region to modulate regulatory protein or gene region function and affect an increase in the amount or rate of expression or activity of an ERK1/2 signaling compound.

Examples of compounds that are activator or agonist of ERK1/2 signaling include: SKF83959 (6-chloro-7,8-dihydroxy-3-methyl-1-(3-methylphenyl)-2,3,4,5-tetrahydro-1H-3-benzazepine; Huang et al, 2012; PLoS ONE 7(11): e49954), PPBP, 4-phenyl-1-(4-phenylbutyl) piperidine; Tan et al, 2010; Neuropharmacology 59:416), CHEMBL1915154, CHEMBL1951219 (Eur J Med Chem. (2012) 50:63), CHEMBL2337988 (J Med Chem. (2013) 56:856) and the peptide CHEMBL3085908 (J Med Chem. (2013) 56:9136.

A preferred activator or agonist of ERK1/2 signaling is one selected from the list consisting of: SKF83959, PPBP, CHEMBL1915154, CHEMBL1951219 and CHEMBL2337988, CHEMBL3085908. In particular embodiments, the activator or agonist of ERK1/2 signaling for component (c) of the combination is SKF83959, CHEMBL1915154 or CHEMBL3085908.

As used herein, the term “activator or agonist of JNK signaling” means a substance that affects an increase in the amount or rate of JNK signaling in a cell. Such a substance can act directly, for example, by binding to the c-Jun N-terminal kinase and increasing the amount or rate of JNK signaling component expression or activity. An agonist JNK signaling can also increase the amount or rate of JNK expression or activity, for example, by binding to JNK in such a way as to enhance or promote JNK signalling events. An activator or agonist of JNK signaling can also act indirectly, for example, by binding to a regulatory molecule or gene region to modulate regulatory protein or gene region function and affect an increase in the amount or rate of expression or activity of an JNK signaling compound.

Examples of compounds that are activator or agonist of JNK signaling include: anisomycin, CHEMBL2393051 (Bioorg Med Chem Lett. (2015) 25:1464), CHEMBL2403796 (Eur J Med. Chem (2014) 84:30) and CHEMBL3318389 (Eur J Med Chem (2014) 84:335), and CHEMBL3325564, CHEMBL3325565, CHEMBL3325566, CHEMBL3325569, CHEMBL3325570 and CHEMBL3325571 (all, J Med Chem (2014) 57:7459).

A preferred activator or agonist of JNK signaling is one selected from the list consisting of: anisomycin, CHEMBL2393051, CHEMBL2403796, CHEMBL3318389, CHEMBL3325564, CHEMBL3325565, CHEMBL3325566, CHEMBL3325569, CHEMBL3325570 and CHEMBL3325571. In particular embodiments, the activator or agonist of JNK signaling for component (c) of the combination is anisomycin, CHEMBL3318389 or CHEMBL3325571.

An activator or agonist of ERK1/2 signaling or an activator or agonist of JNK signaling can be, for example, a naturally or non-naturally occurring macromolecule, such as a polypeptide, peptide, peptidomimetic, nucleic acid, carbohydrate or lipid. An activator or agonist of ERK1/2 signaling or an activator or agonist of JNK signaling further can be an antibody, or antigen-binding fragment thereof, such as a mono-clonal antibody, humanized antibody, chimeric antibody, minibody, bi-functional antibody, single chain antibody (scFv), variable region fragment (Fv or Fd), Fab or F(ab)2. An activator or agonist of ERK1/2 signaling or an activator or agonist of JNK signaling can also be a polyclonal antibody. An activator or agonist of ERK1/2 signaling or an activator or agonist of JNK signaling further can be a partially, or completely synthetic derivative, analog or mimetic of a naturally occurring macromolecule, or a small organic or inorganic molecule.

Activators or agonists of ERK1/2 signaling in accordance with the present invention are also expression constructs expressing ERK1/2 components or functional fragments thereof, and the activators or agonists of JNK signaling in accordance with the present invention are also expression constructs expressing JNK components or functional fragments thereof. The term “expression construct” means any double-stranded DNA or double-stranded RNA designed to transcribe an RNA, e.g., a construct that contains at least one promoter operably linked to a downstream gene or coding region of interest (e.g., a cDNA or genomic DNA fragment that encodes a protein, or any RNA of interest). Transfection or trans-formation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus, or further embodiments described under “expression vector” below. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct. Further, the term expression construct or vector is intended to also include instances wherein the cell utilized for the assay already endogenously comprises such DNA sequence.

Another aspect of the present invention pertains to a pharmaceutical composition for use in the prevention or treatment of a tumor disease. The pharmaceutical composition of the invention comprises an inhibitor (or activator, as applicable) of the respective gene, or a combination as described herein above, and a pharmaceutical acceptable carrier and/or excipient. As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Kolliphor® EL (formerly Cremophor EL™; BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an inhibitor or antagonist, or activator or agonist, as applicable, of the respective gene) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, as well as comprising a compound or combination of the invention (eg an inhibitor or antagonist, or activator or agonist, as applicable, of the respective gene) generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

Furthermore, the compounds or combinations of the invention (eg an inhibitor or antagonist, or activator or agonist, as applicable, of the respective gene) can be administrated rectally. A rectal composition can be any rectally acceptable dosage form including, but not limited to, cream, gel, emulsion, enema, suspension, suppository, and tablet. One preferred dosage form is a suppository having a shape and size designed for introduction into the rectal orifice of the human body. A suppository usually softens, melts, or dissolves at body temperature. Suppository excipients include, but are not limited to, theobroma oil (cocoa butter), glycerinated gelatin, hydrogenated vegetable oils, mixtures of polyethylene glycols of various molecular weights, and fatty acid esters of polyethylene glycol.

For administration by inhalation, the compounds of combinations of the invention (eg an inhibitor or antagonist, or activator or agonist, as applicable, of the respective gene) are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient (eg an inhibitor or antagonist, or activator or agonist, as applicable, of the respective gene). Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially (including liposomes targeted to infected cells with monoclonal antibodies to viral antigens) can also be used as pharmaceutically acceptable carriers. These can be prepared according to methods known to those skilled in the art.

It is especially advantageous to formulate oral, rectal or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. The pharmaceutical compositions can be included in a container, pack, or dispenser together with instructions for administration.

In the context of the invention, an effective amount (eg, a therapeutically effective amount) of the respective compound (eg inhibitor or antagonist or of the activator or agonist), or the pharmaceutical composition, can be one that will elicit the biological, physiological, pharmacological, therapeutic or medical response of a cell, tissue, system, body, animal, individual, patient or human that is being sought by the researcher, scientist, pharmacologist, pharmacist, veterinarian, medical doctor, or other clinician, e.g., lessening of the effects/symptoms of a disorder, disease or condition, such as a proliferative disorder or disease, for example, a cancer or tumor, or killing or inhibiting growth of a proliferating cell, such as a tumor cell. The effective amount can be determined by standard procedures, including those described above and below.

In accordance with all aspects and embodiments of the medical uses and methods of treatment provided herein, the effective amount administered at least once to a subject in need of said compound, for example when such compound is a protein like an antibody, is between about 0.01 mg/kg and about 100 mg/kg per administration, such as between about 1 mg/kg and about 10 mg/kg per administration. In some embodiments, the effective amount administered at least once to said subject of said compound is between about 0.01 mg/kg and about 0.1 mg/kg per administration, between about 0.1 mg/kg and about 1 mg/kg per administration, between about 1 mg/kg and about 5 mg/kg per administration, between about 5 mg/kg and about 10 mg/kg per administration, between about 10 mg/kg and about 50 mg/kg per administration, or between about 50 mg/kg and about 100 mg/kg per administration.

In accordance with all aspects of the medical uses and methods of treatment provided herein, the effective amount administered at least once to said subject of said compound, for example when such compound is a nucleic acid like, is between about 0.01 μg/kg and about 1000 μg/kg per administration. In some embodiments, the effective amount administered at least once to said subject of said compound is between about 0.05 μg/kg and about 500 μg/kg per administration, between about 0.1 μg/kg and about 100 μg/kg per administration, between about 10 μg/kg and about 50 μg/kg per administration, between about 50 μg/kg and about 100 μg/kg per administration, or between about 100 μg/kg and about 250 μg/kg per administration, or between about 250 μg/kg and about 500 μg/kg per administration.

For the prevention or treatment of disease, the appropriate dosage of compound (e.g. antibody or nucleic acid), or a pharmaceutical composition comprised thereof, will depend on the type of disease to be treated, the severity and course of the disease, whether said compound and/or pharmaceutical composition is administered for preventive or therapeutic purposes, previous therapy, the patient's clinical history, age, size/weight and response to said compound and/or pharmaceutical composition, and the discretion of the attending physician. The compound and/or pharmaceutical composition is suitably administered to the patient at one time or over a series of treatments. If such compound and/or pharmaceutical composition is administered over a series of treatments, the total number of administrations for a given course of treatment may consist of a total of about 2, 3, 4, 5, 6, 7, 8, 9, 10 or more than about 10 treatments. For example, a treatment may be given once every day (or 2, 3 or 4 times a day) for a week, a month or even several months. In certain embodiments, the course of treatment may continue indefinitely.

The amount administered will depend on variables such as the type and extent of disease or indication to be treated, the overall health, age, size/weight of the patient, the in vivo potency of the compound, the pharmaceutical composition, and the route of administration. The initial dosage can be increased beyond the upper level in order to rapidly achieve the desired bloodlevel or tissue level. Alternatively, the initial dosage can be smaller than the optimum, and the daily dosage may be progressively increased during the course of treatment. Human dosage can be optimized, e.g., in a conventional Phase I dose escalation study designed to run from relatively low initial doses, for example from about 0.01 mg/kg to about 20 mg/kg of antibody. Dosing frequency can vary, depending on factors such as route of administration, dosage amount and the disease being treated. Exemplary dosing frequencies are once per day, once per week and once every two weeks. Formulation of a compound or combination of the present invention, is within the ordinary skill in the art. In some embodiments of the invention such an antibody or nucleic acid is lyophilized and reconstituted in buffered saline at the time of administration. The a compound, combination and/or pharmaceutical composition of the present invention may further result in a reduced relapsing of the disease to be treated or reduce the incidence of drug resistance or increase the time until drug resistance is developing; and in the case of cancer may result in an increase in the period of progression-free survival and/or overall survival.

In view of the above, it will be appreciated that the present invention also relates to the following itemised embodiments:

Item 1. A method for reducing resistance of a tumor cell to an immune response, the method comprising a step of contacting the tumor cell with a modulator of tumor resistance selected from an

(a) inhibitor or antagonist of CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, or

(b) an activator or agonist of ENPP2, GRM6, GRK5, OR1G1, CCR2, ADMR, GRM4, MASS1, CXCL9, CXCR3, or OR1D4.

Item 2. The method according to item 1, wherein the resistance of a tumor cell to an immune response is a resistance of the tumor cell to a T cell mediated immune response.

Item 3. The method according to item 1, wherein said modulator of tumor resistance is

(a) An inhibitor or antagonist of expression, protein function, or signaling of a protein selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, or

(b) an activator or agonist of the expression, protein function, or signaling of a protein selected from ENPP2, GRM6, GRK5, OR1G1, CCR2, ADMR, GRM4, MASS1, CXCL9, CXCR3, or OR1D4.

Item 4. The method according to item 2, comprising a step of contacting the tumor cell with at least one additional compound effective in the treatment of cancer, preferably wherein the at least one additional compound effective in the treatment of cancer is one or more modulators of tumor resistance selected from

(a) An inhibitor or antagonist of expression, protein function, or signaling of a protein selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, and/or

(b) an activator or agonist of the expression, protein function, or signaling of a protein selected from ENPP2, GRM6, GRK5, OR1G1, CCR2, ADMR, GRM4, MASS1, CXCL9, CXCR3, or OR1D4.

Item 5. The method according to item 2, wherein said tumor cell is characterized by a detectable cell surface expression of CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, OR2J2 before contacting the tumor cell with the corresponding modulator of tumor resistance.

Item 6. The method according to item 3, wherein said inhibitor of CCR9-T-cell interaction is an inhibitor of CCR9 mediated STAT1 impairment in T-cells.

Item 7. A method for treating a tumor disease in a patient, wherein said tumor disease is characterized by a resistance of said tumor against immune responses, the method comprising a step of

(a) Inhibiting or antagonizing in said patient CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, or

(b) Activating or agonizing ENPP2, GRM6, GRK5, OR1G1, CCR2, ADMR, GRM4, MASS1, CXCL9, CXCR3, or OR1D4.

Item 8. A method for aiding a patient's immune response against a tumor disease comprising a step of

(a) Inhibiting or antagonizing in said patient CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, or

(b) Activating or agonizing ENPP2, GRM6, GRK5, OR1G1, CCR2, ADMR, GRM4, MASS1, CXCL9, CXCR3, or OR1D4.

Item 9. The method according to item 7 or 8, comprising a step of administering to said patient a therapeutically effective amount of a modulator of tumor resistance selected from an

(a) inhibitor or antagonist of CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, or

(b) an activator or agonist of ENPP2, GRM6, GRK5, OR1G1, CCR2, ADMR, GRM4, MASS1, CXCL9, CXCR3, or OR1D4.

Item 10. The method according to item 1, wherein said inhibitor or antagonist, or said activator or agonist, is a compound is selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9, a carbohydrate such as a polysaccharide or oligosaccharide and the like, including variants or derivatives thereof, a lipid such as a fatty acid and the like, including variants or derivatives thereof; or a small organic molecules including but not limited to small molecule ligands, small cell-permeable molecules, and peptidomimetic compounds.

Item 11. The method according to item 1, wherein said tumor cell, tumor or tumor disease is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia.

Item 12. A method for identifying a therapeutic compound suitable for the treatment of a tumor disease, the method comprising the steps of

(a) Providing a first cell expressing a protein on the cellular surface, wherein the protein is selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, or is selected from ENPP2, GRM6, GRK5, OR1G1, CCR2, ADMR, GRM4, MASS1, CXCL9, CXCR3, or OR1D4,

(b) Contacting said first cell with a candidate compound,

(c) And/or, contacting subsequent to step (b) said first cell with a cytotoxic T-lymphocyte (CTL), and

(d) Determining subsequent to step (b) and/or (c) protein expression/function in said first cell, wherein a differential protein expression in said first cell contacted with the candidate compound compared to said first cell not contacted with said candidate compound indicates that the candidate compound is a therapeutic compound suitable for the treatment of a tumor disease; and/or

(e) Determining subsequent to step (c) cytotoxicity of said CTL against said first cell, wherein an enhanced cytotoxicity of said CTL against said first cell contacted with the candidate compound compared to the cytotoxicity of said CTL against said first cell not contacted with the candidate compound indicates that the candidate compound is a therapeutic compound suitable for the treatment of a tumor disease.

Item 13. The method according to item 12, wherein a reduced protein expression/function of a protein selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, OR2J2 in said first cell contacted with the candidate compound compared to said first cell not contacted with said candidate compound indicates that the candidate compound is a therapeutic compound suitable for the treatment of a tumor disease.

Item 14. The method according to item 12, wherein an increased protein expression/function of a protein selected from ENPP2, GRM6, GRK5, OR1G1, CCR2, ADMR, GRM4, MASS1, CXCL9, CXCR3, or OR1D4 in said first cell contacted with the candidate compound compared to said first cell not contacted with said candidate compound indicates that the candidate compound is a therapeutic compound suitable for the treatment of a tumor disease.

Item 15. The method according to item 12, wherein said first cell is a cell resistant to cytotoxicity mediated by T-lymphocytes, preferably a tumor derived cell.

Item 16. The method according to item 12, wherein said candidate compound is selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct, a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

Item 17. A method for diagnosing in a patient a resistance of a tumor disease against T cell mediated immune responses, the method comprising a step of

(a) determining expression of a protein selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5, TRAR3, OR2J2 in a tumor cell from the tumor of the patient, wherein a detectable or increased expression of any one of the proteins in the tumor cell compared to a negative control is indicative for a resistance of the tumor disease against T cell mediated immune responses; or

(b) determining expression of a protein selected from ENPP2, GRM6, GRK5, OR1G1, CCR2, ADMR, GRM4, MASS1, CXCL9, CXCR3, or OR1D4 in a tumor cell from the tumor of the patient, wherein a reduced expression of any one of the proteins in the tumor cell compared to a negative control is indicative for a resistance of the tumor disease against T cell mediated immune responses.

Item 18. The method according to item 17, comprising a preceding step of obtaining a tumor cell from the patient.

The present invention will now be further described in the following examples with reference to the accompanying figures and sequences, nevertheless, without being limited thereto. For the purposes of the present invention, all references as cited herein are incorporated by reference in their entireties.

### IN THE FIGURES

FIG. 1: Heat map representation of potential positive and negative immune modulators identified from the RNAi screens Differential scores were used to identify positive immune modulators (yellow) the knockdown of which enhance CTL-mediated cell killing and negative immune modulators (blue) the knockdown of which reduce CTL-mediated cell killing. Differential scores prior to filtering are shown for all genes tested in the 3 different screens (see Materials and Methods). Selected representative clusters of high-confidence hits are displayed herein.

FIG. 2: CCR9 knockdown sensitizes tumor cells to immune attack A MCF7 cells were transfected with the described siRNA sequences and harvested after 72 h for mRNA and protein estimation using RT-PCR (upper) and immunoblot (lower) analysis, respectively. GAPDH and beta-actin were used as controls for RNA and protein normalization, respectively. B Luc-CTL cytotoxicity assay with PBMC-derived CTLs and bi-specific Ab as effector population and MCF7 as target cells, which were transfected with individual (s1-s4) or pooled CCR9 siRNA sequences. PD-L1 and non-specific control siRNAs were used as positive and negative controls, respectively, for CTL-mediated cytotoxicity. C, D Cr-release assay showing % specific lysis of MCF7 cells by survivin-specific T cells at different ratios upon CCR9 knockdown (C) or overexpression (D). MCF7 cells were transfected with either CCR9 siRNA s1 (Δ), pooled siRNA sequences (∘), positive control PD-L1 (□), and non-specific control siRNA (▪) (C) or with pCMV6-AC-His control vector (▪) and pCMV6-AC-His-CCR9 expression construct (∘) (D) 72 h prior to the assay. E Cr-release assay showing % specific lysis of MDA-MB-231 breast tumor cell line by survivin-specific T cells at different ratios upon CCR9 knockdown (∘) in comparison to the control knockdown (▪). F, G Cr-release assay showing lysis of patient-derived melanoma cells (M579-A2) by tumor-infiltrating lymphocytes (TIL 412) (F) or lysis of PANC-1 pancreatic adenocarcinoma cells by patient-derived pancreatic TIL 53 (G) at different E:T ratios upon CCR9 (∘) or control (▪) knockdown. Data information: All experiments were performed in triplicates and are representative of at least three independent experiments. Error bars denote ±SEM, and statistical significance was calculated using the unpaired, two-tailed Student's t-test.

FIG. 3: Tumor-specific CCR9 impedes Th1-type immune response A, B ELISpot assay showing IFN-γ (A) and granzyme B (B) secretion by survivin-specific T cells, as spot numbers, upon CCR9 knockdown (black bars) in MCF7 cells compared to the control knockdown (white bars). T cells (TC) alone (grey bars) were used as control for background spot numbers. C Luminex assay showing cytokine levels in the supernatant from the coculture of survivin-specific TC and either CCR9^{hi} MCF7 (transfected with CCR9-specific siRNA) or CCR9^{lo} MCF7 (transfected with control siRNA) cells. D Phosphoplex analysis showing the phospho-STAT levels in survivin-specific TC upon encountering CCR9^{hi} or CCR9^{lo} MCF7 cells. Log 2 ratio of mean fluorescent intensity (MFI) of the respective analytes to the unstimulated TC is plotted herein. E Immunoblot analysis showing the phospho-STAT1 levels in the CCR9^{hi}-treated, CCR9^{lo}-treated or unstimulated TC using the phospho-specific STAT1 (pTyr701) antibody. Beta-actin was used as the loading control. Data information: In all the cases, experiments were performed in triplicate with at least two independent repeats. Mean±SEM are shown herein, unless stated otherwise, with statistical significance assessed using unpaired, two-tailed Student's t-test. Source data are available online for this figure.

FIG. 4: Tumor-specific CCR9 interacts directly with T cells inducing prominent changes in the gene expression signature A ELISA showing CCL25 levels in cell lysates from indicated tumor cell lines. CCR9 knockdown (k.d.) in MCF7 cells was achieved using specific shRNA (see Materials and Methods). B Cr-release assay showing % specific lysis of MCF7 cells by survivin TC upon CCL25 (□) or CCR9 (∘) inhibition using specific siRNAs in comparison to the control siRNA (▪). Mean±SEM are depicted herein. C MCF7 cells were transfected with control or CCR9-specific siRNAs, and 48 h later, the supernatants (CCR91^{lo} or CCR9^{hi} SSN, respectively) were used to culture survivin TCs overnight. Supernatant-treated TCs were then used as effector cells against CCR9^{lo} or CCR9^{hi} MCF7 tumor cells in the Cr-release assay along with wild-type MCF7 cells. Mean±SEM are depicted herein. D Cr-release assay showing % specific lysis of MCF7 cells that were pre-treated with or without pertussis toxin (PTX), or knocked down for CCR9 using specific siRNA. Mean±SEM are depicted herein. E, F MCF7 cells transfected with control siRNA (CCR9^{hi}) or CCR9 siRNA (CCR9^{lo}) were cocultured with survivin TCs for 12 h. Gene microarray was performed with the total RNA extracted from purified T cells after the coculture. Volcano plot (E) illustrating fold change (FC; log 2) in gene expression intensities compared with P-value (−log 2) between CCR9^{hi}- and CCR9^{lo}-treated TCs. Horizontal bar at y=4.32 represents a statistical significance of P=0.05 (genes in gray below this line did not reach significance). Log FC cutoff at +0.5 is represented by the vertical lines. Heatmap representation of the top upregulated (Log FC>0.5) and downregulated (Log FC<−0.85) genes (F) with P≤0.05. Individual replicates per sample group are shown herein.

FIG. 5: In vivo inhibition of CCR9 significantly reduces tumor outgrowth in response to adoptive TIL therapy A Cr-release assay showing TIL 209-mediated lysis of CCR9⁺ M579-A2 (transduced with control shRNA) or CCR9⁻ M579-A2 cells (transduced with CCR9-specific shRNA). Curves represent mean±SEM. B Scheme for the in vivo mouse experiment involving the s.c. injection of CCR9+(shControl) or CCR9 (shCCR9) M579-A2 tumor cells in the left and right flank, respectively, of the NSG mice. Following this, at d2 and d9, mice received i.v. injection of TIL 209 in PBS (n=7) or PBS alone (control group for tumor growth; n=3) and measured for tumor growth. C, D Tumor growth curves showing mean±SEM tumor volume of CCR9⁺ or CCR9⁻ M579-A2 tumors in TIL-treated mice (C) or the PBS alone group (D). Statistical difference was calculated using the unpaired one-sided Mann-Whitney U-test.

FIG. 6: Altered signaling cascades in MCF7 tumor cells upon CCR9 knockdown. MCF7 cells were reverse transfected with control or CCR9-specific siRNA and after 72 h protein lysates were used for phospho-plex analysis of the major transcription factors indicated on x-axis (studied phopho-sites are indicated in brackets). Statistical differences between the two groups were analyzed using student's two-sided t-test, n=3. Error bars represent SEM.

### IN THE SEQUENCES: [INCLUDED TO BE CONSISTENT IN CASE WE PROSECUTE A CCR9 ASPECT OUT OF HERE]

SEQ ID NO: 1 shows Homo sapiens Isoform 1 of C-C chemokine
receptor type 9 CCR9:
MTPTDFTSPIPNMADDYGSESTSSMEDYVNFNFTDFYCEKNNVRQFASHFLPPLYWLVFIVG
ALGNSLVILVYWYCTRVKTMTDMFLLNLAIADLLFLVTLPFWAIAAADQWKFQTFMCKVVNS
MYKMNFYSCVLLIMCISVDRYIAIAQAMRAHTWREKRLLYSKMVCFTIWVLAAALCIPEILY
SQIKEESGIAICTMVYPSDESTKLKSAVLTLKVILGFFLPFVVMACCYTIIIHTLIQAKKSS
KHKALKVTITVLTVFVLSQFPYNCILLVQTIDAYAMFISNCAVSTNIDICFQVTQTIAFFHS
CLNPVLYVFVGERFRRDLVKTLKNLGCISQAQWVSFTRREGSLKLSSMLLETTSGALSL
SEQ ID NO: 2 shows Homo sapiens Isoform 2 of C-C chemokine
receptor type 9 CCR9:
MADDYGSESTSSMEDYVNFNFTDFYCEKNNVRQFASHFLPPLYWLVFIVGALGNSLVILVYW
YCTRVKTMTDMFLLNLAIADLLFLVTLPFWAIAAADQWKFQTFMCKVVNSMYKMNFYSCVLL
IMCISVDRYIAIAQAMRAHTWREKRLLYSKMVCFTIWVLAAALCIPEILYSQIKEESGIAIC
TMVYPSDESTKLKSAVLTLKVILGFFLPFVVMACCYTIIIHTLIQAKKSSKHKALKVTITVL
TVFVLSQFPYNCILLVQTIDAYAMFISNCAVSTNIDICFQVTQTIAFFHSCLNPVLYVFVGE
RFRRDLVKTLKNLGCISQAQWVSFTRREGSLKLSSMLLETTSGALSL
SEQ ID NO: 3 shows Homo sapiens C-C motif chemokine receptor 9
(CCR9), isoform 1, mRNA:
GCTTCCTTTCTCGTGTTGTTATCGGGTAGCTGCCTGCTCAGAACCCACAAAGCCTGCCCCTC
ATCCCAGGCAGAGAGCAACCCAGCTCTTTCCCCAGACACTGAGAGCTGGTGGTGCCTGCTGT
CCCAGGGAGAGTTGCATCGCCCTCCACAGAGCAGGCTTGCATCTGACTGACCCACCATGACA
CCCACAGACTTCACAAGCCCTATTCCTAACATGGCTGATGACTATGGCTCTGAATCCACATC
TTCCATGGAAGACTACGTTAACTTCAACTTCACTGACTTCTACTGTGAGAAAAACAATGTCA
GGCAGTTTGCGAGCCATTTCCTCCCACCCTTGTACTGGCTCGTGTTCATCGTGGGTGCCTTG
GGCAACAGTCTTGTTATCCTTGTCTACTGGTACTGCACAAGAGTGAAGACCATGACCGACAT
GTTCCTTTTGAATTTGGCAATTGCTGACCTCCTCTTTCTTGTCACTCTTCCCTTCTGGGCCA
TTGCTGCTGCTGACCAGTGGAAGTTCCAGACCTTCATGTGCAAGGTGGTCAACAGCATGTAC
AAGATGAACTTCTACAGCTGTGTGTTGCTGATCATGTGCATCAGCGTGGACAGGTACATTGC
CATTGCCCAGGCCATGAGAGCACATACTTGGAGGGAGAAAAGGCTTTTGTACAGCAAAATGG
TTTGCTTTACCATCTGGGTATTGGCAGCTGCTCTCTGCATCCCAGAAATCTTATACAGCCAA
ATCAAGGAGGAATCCGGCATTGCTATCTGCACCATGGTTTACCCTAGCGATGAGAGCACCAA
ACTGAAGTCAGCTGTCTTGACCCTGAAGGTCATTCTGGGGTTCTTCCTTCCCTTCGTGGTCA
TGGCTTGCTGCTATACCATCATCATTCACACCCTGATACAAGCCAAGAAGTCTTCCAAGCAC
AAAGCCCTAAAAGTGACCATCACTGTCCTGACCGTCTTTGTCTTGTCTCAGTTTCCCTACAA
CTGCATTTTGTTGGTGCAGACCATTGACGCCTATGCCATGTTCATCTCCAACTGTGCCGTTT
CCACCAACATTGACATCTGCTTCCAGGTCACCCAGACCATCGCCTTCTTCCACAGTTGCCTG
AACCCTGTTCTCTATGTTTTTGTGGGTGAGAGATTCCGCCGGGATCTCGTGAAAACCCTGAA
GAACTTGGGTTGCATCAGCCAGGCCCAGTGGGTTTCATTTACAAGGAGAGAGGGAAGCTTGA
AGCTGTCGTCTATGTTGCTGGAGACAACCTCAGGAGCACTCTCCCTCTGAGGGGTCTTCTCT
GAGGTGCATGGTTCTTTTGGAAGAAATGAGAAATACAGAAACAGTTTCCCCACTGATGGGAC
CAGAGAGAGTGAAAGAGAAAAGAAAACTCAGAAAGGGATGAATCTGAACTATATGATTACTT
GTAGTCAGAATTTGCCAAAGCAAATATTTCAAAATCAACTGACTAGTGCAGGAGGCTGTTGA
TTGGCTCTTGACTGTGATGCCCGCAATTCTCAAAGGAGGACTAAGGACCGGCACTGTGGAGC
ACCCTGGCTTTGCCACTCGCCGGAGCATCAATGCCGCTGCCTCTGGAGGAGCCCTTGGATTT
TCTCCATGCACTGTGAACTTCTGTGGCTTCAGTTCTCATGCTGCCTCTTCCAAAAGGGGACA
CAGAAGCACTGGCTGCTGCTACAGACCGCAAAAGCAGAAAGTTTCGTGAAAATGTCCATCTT
TGGGAAATTTTCTACCCTGCTCTTGAGCCTGATAACCCATGCCAGGTCTTATAGATTCCTGA
TCTAGAACCTTTCCAGGCAATCTCAGACCTAATTTCCTTCTGTTCTCCTTGTTCTGTTCTGG
GCCAGTGAAGGTCCTTGTTCTGATTTTGAAACGATCTGCAGGTCTTGCCAGTGAACCCCTGG
ACAACTGACCACACCCACAAGGCATCCAAAGTCTGTTGGCTTCCAATCCATTTCTGTGTCCT
GCTGGAGGTTTTAACCTAGACAAGGATTCCGCTTATTCCTTGGTATGGTGACAGTGTCTCTC
CATGGCCTGAGCAGGGAGATTATAACAGCTGGGTTCGCAGGAGCCAGCCTTGGCCCTGTTGT
AGGCTTGTTCTGTTGAGTGGCACTTGCTTTGGGTCCACCGTCTGTCTGCTCCCTAGAAAATG
GGCTGGTTCTTTTGGCCCTCTTCTTTCTGAGGCCCACTTTATTCTGAGGAATACAGTGAGCA
GATATGGGCAGCAGCCAGGTAGGGCAAAGGGGTGAAGCGCAGGCCTTGCTGGAAGGCTATTT
ACTTCCATGCTTCTCCTTTTCTTACTCTATAGTGGCAACATTTTAAAAGCTTTTAACTTAGA
GATTAGGCTGAAAAAAATAAGTAATGGAATTCACCTTTGCATCTTTTGTGTCTTTCTTATCA
TGATTTGGCAAAATGCATCACCTTTGAAAATATTTCACATATTGGAAAAGTGCTTTTTAATG
TGTATATGAAGCATTAATTACTTGTCACTTTCTTTACCCTGTCTCAATATTTTAAGTGTGTG
CAATTAAAGATCAAATAGATACATT
SEQ ID NO: 4 shows Homo sapiens C-C motif chemokine receptor 9
(CCR9), isoform 2 mRNA:
GCTTCCTTTCTCGTGTTGTTATCGGGTAGCTGCCTGCTCAGAACCCACAAAGCCTGCCCCTC
ATCCCAGGCAGAGAGCAACCCAGCTCTTTCCCCAGACACTGAGAGCTGGTGGTGCCTGCTGT
CCCAGGGAGAGTTGCATCGCCCTCCACAAGCCCTATTCCTAACATGGCTGATGACTATGGCT
CTGAATCCACATCTTCCATGGAAGACTACGTTAACTTCAACTTCACTGACTTCTACTGTGAG
AAAAACAATGTCAGGCAGTTTGCGAGCCATTTCCTCCCACCCTTGTACTGGCTCGTGTTCAT
CGTGGGTGCCTTGGGCAACAGTCTTGTTATCCTTGTCTACTGGTACTGCACAAGAGTGAAGA
CCATGACCGACATGTTCCTTTTGAATTTGGCAATTGCTGACCTCCTCTTTCTTGTCACTCTT
CCCTTCTGGGCCATTGCTGCTGCTGACCAGTGGAAGTTCCAGACCTTCATGTGCAAGGTGGT
CAACAGCATGTACAAGATGAACTTCTACAGCTGTGTGTTGCTGATCATGTGCATCAGCGTGG
ACAGGTACATTGCCATTGCCCAGGCCATGAGAGCACATACTTGGAGGGAGAAAAGGCTTTTG
TACAGCAAAATGGTTTGCTTTACCATCTGGGTATTGGCAGCTGCTCTCTGCATCCCAGAAAT
CTTATACAGCCAAATCAAGGAGGAATCCGGCATTGCTATCTGCACCATGGTTTACCCTAGCG
ATGAGAGCACCAAACTGAAGTCAGCTGTCTTGACCCTGAAGGTCATTCTGGGGTTCTTCCTT
CCCTTCGTGGTCATGGCTTGCTGCTATACCATCATCATTCACACCCTGATACAAGCCAAGAA
GTCTTCCAAGCACAAAGCCCTAAAAGTGACCATCACTGTCCTGACCGTCTTTGTCTTGTCTC
AGTTTCCCTACAACTGCATTTTGTTGGTGCAGACCATTGACGCCTATGCCATGTTCATCTCC
AACTGTGCCGTTTCCACCAACATTGACATCTGCTTCCAGGTCACCCAGACCATCGCCTTCTT
CCACAGTTGCCTGAACCCTGTTCTCTATGTTTTTGTGGGTGAGAGATTCCGCCGGGATCTCG
TGAAAACCCTGAAGAACTTGGGTTGCATCAGCCAGGCCCAGTGGGTTTCATTTACAAGGAGA
GAGGGAAGCTTGAAGCTGTCGTCTATGTTGCTGGAGACAACCTCAGGAGCACTCTCCCTCTG
AGGGGTCTTCTCTGAGGTGCATGGTTCTTTTGGAAGAAATGAGAAATACAGAAACAGTTTCC
CCACTGATGGGACCAGAGAGAGTGAAAGAGAAAAGAAAACTCAGAAAGGGATGAATCTGAAC
TATATGATTACTTGTAGTCAGAATTTGCCAAAGCAAATATTTCAAAATCAACTGACTAGTGC
AGGAGGCTGTTGATTGGCTCTTGACTGTGATGCCCGCAATTCTCAAAGGAGGACTAAGGACC
GGCACTGTGGAGCACCCTGGCTTTGCCACTCGCCGGAGCATCAATGCCGCTGCCTCTGGAGG
AGCCCTTGGATTTTCTCCATGCACTGTGAACTTCTGTGGCTTCAGTTCTCATGCTGCCTCTT
CCAAAAGGGGACACAGAAGCACTGGCTGCTGCTACAGACCGCAAAAGCAGAAAGTTTCGTGA
AAATGTCCATCTTTGGGAAATTTTCTACCCTGCTCTTGAGCCTGATAACCCATGCCAGGTCT
TATAGATTCCTGATCTAGAACCTTTCCAGGCAATCTCAGACCTAATTTCCTTCTGTTCTCCT
TGTTCTGTTCTGGGCCAGTGAAGGTCCTTGTTCTGATTTTGAAACGATCTGCAGGTCTTGCC
AGTGAACCCCTGGACAACTGACCACACCCACAAGGCATCCAAAGTCTGTTGGCTTCCAATCC
ATTTCTGTGTCCTGCTGGAGGTTTTAACCTAGACAAGGATTCCGCTTATTCCTTGGTATGGT
GACAGTGTCTCTCCATGGCCTGAGCAGGGAGATTATAACAGCTGGGTTCGCAGGAGCCAGCC
TTGGCCCTGTTGTAGGCTTGTTCTGTTGAGTGGCACTTGCTTTGGGTCCACCGTCTGTCTGC
TCCCTAGAAAATGGGCTGGTTCTTTTGGCCCTCTTCTTTCTGAGGCCCACTTTATTCTGAGG
AATACAGTGAGCAGATATGGGCAGCAGCCAGGTAGGGCAAAGGGGTGAAGCGCAGGCCTTGC
TGGAAGGCTATTTACTTCCATGCTTCTCCTTTTCTTACTCTATAGTGGCAACATTTTAAAAG
CTTTTAACTTAGAGATTAGGCTGAAAAAAATAAGTAATGGAATTCACCTTTGCATCTTTTGT
GTCTTTCTTATCATGATTTGGCAAAATGCATCACCTTTGAAAATATTTCACATATTGGAAAA
GTGCTTTTTAATGTGTATATGAAGCATTAATTACTTGTCACTTTCTTTACCCTGTCTCAATA
TTTTAAGTGTGTGCAATTAAAGATCAAATAGATACATT
SEQ ID NO: 5 shows a CCR9-specific shRNA hairpin:
ACCGGGCCAGTGGAGGTCTTTGTTCTGTTAATATTCATAGCAGAACAAGGACCTTCACTGGC
TTTT

### IN THE EXAMPLES

### Example 1: Validation of Immune-Modulatory Function of CCR9

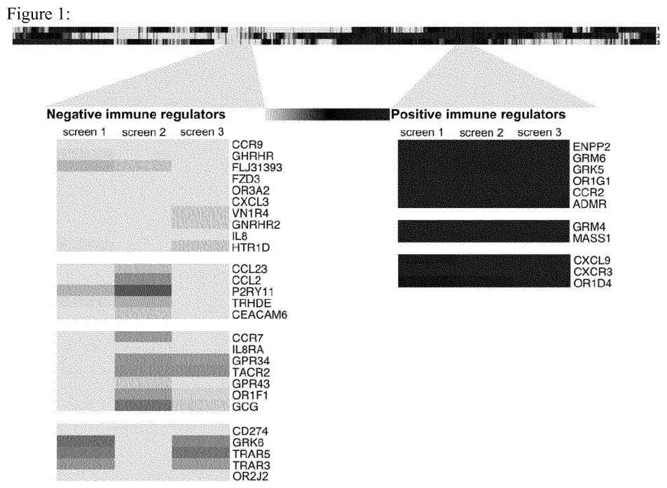

An siRNA screen for immunomodulatory factors was performed as in Khandelwal N et al 2015. FIG. 1 shows the screening results and selected new candidates. For exemplary functional validation of the screening approach, the C-C chemokine receptor type 9 (CCR9) was chosen as it was found to be highly immunosuppressive in all the three screens despite the divergent biological background, inhibiting T cell function in an antigen-dependent as well as antigen-independent manner (FIG. 1). CCR9 is a chemokine receptor involved in immune cell trafficking (Kunkel et al, 2000; Uehara et al, 2002) and is expressed on tolerogenic plasmacytoid dendritic cells (Hadeiba et al, 2008). So far, an implication of CCR9 in T cell function or tumor-immune resistance has not been reported.

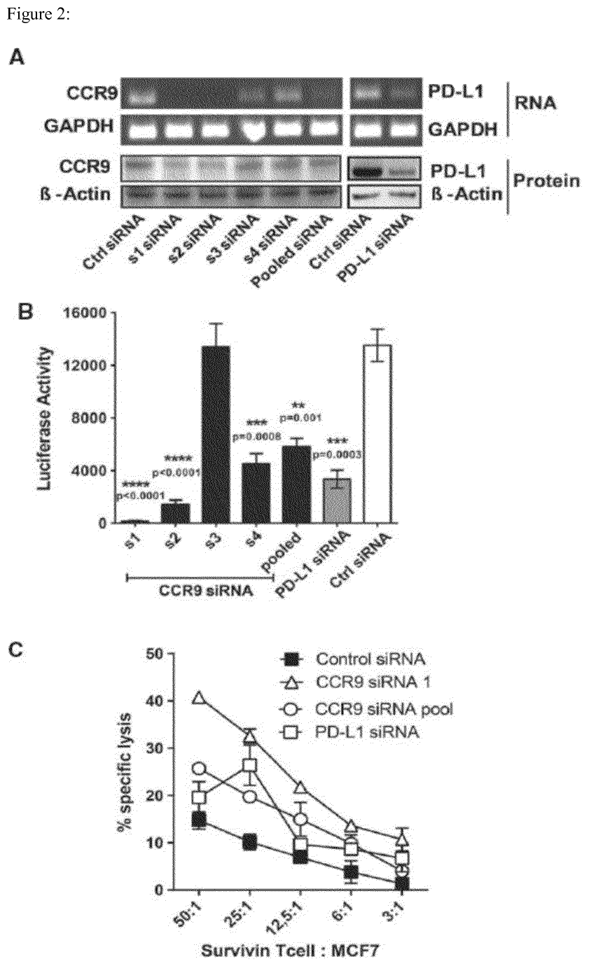

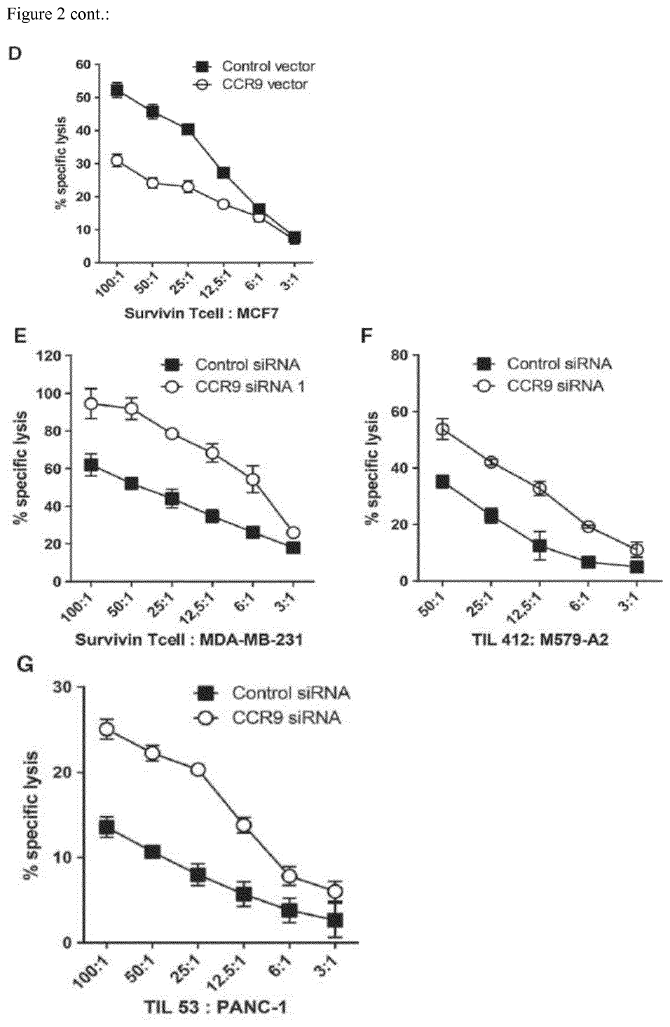

The mRNA and protein knockdown efficiency of single siRNAs within the CCR9 siRNA pool correlated well with the functional effect on T cell cytotoxicity (FIGS. 2A and B), while none of the CCR9 siRNAs influenced cell viability. Surface expression of CCR9 on MCF7 cells was also found to be reduced by 50% in flow cytometry staining using CCR9 s1 siRNA. Knockdown of CCR9 using siRNA markedly increased MCF7 lysis by survivin-specific CTL (FIG. 2C) in the classical chromium-release assay.

Conversely, overexpression of CCR9 inhibited tumor lysis, demonstrating that CCR9 expression enables immune escape of cancer cells (FIG. 2D). CCR9 inhibition in MDA-MB-231 metastatic breast cancer cell line also resulted in marked increase in immune-mediated tumor lysis (FIG. 2E). To explore the broad applicability of CCR9-mediated immune suppression in different tumor entities under clinical setting, the inventors next silenced CCR9 in patient-derived primary melanoma cells (M579-A2 cells) and co-cultured them with HLA-matched tumor-infiltrating lymphocytes (TIL; clone 412) derived from melanoma patient and found a remarkable increase in melanoma cell lysis upon CCR9 knockdown in comparison to the control knockdown (FIG. 2F). Similarly, HLA-matched TIL cultures (TIL 53) from pancreatic adenocarcinoma patients recognized and lysed PANC-1 pancreatic cancer cells more effectively upon CCR9 knockdown as shown in FIG. 2G, stressing that CCR9-mediated immune suppression may be a clinically relevant phenomenon in multiple tumor entities.

### Example 2: CCR9 Influence on CTL Function

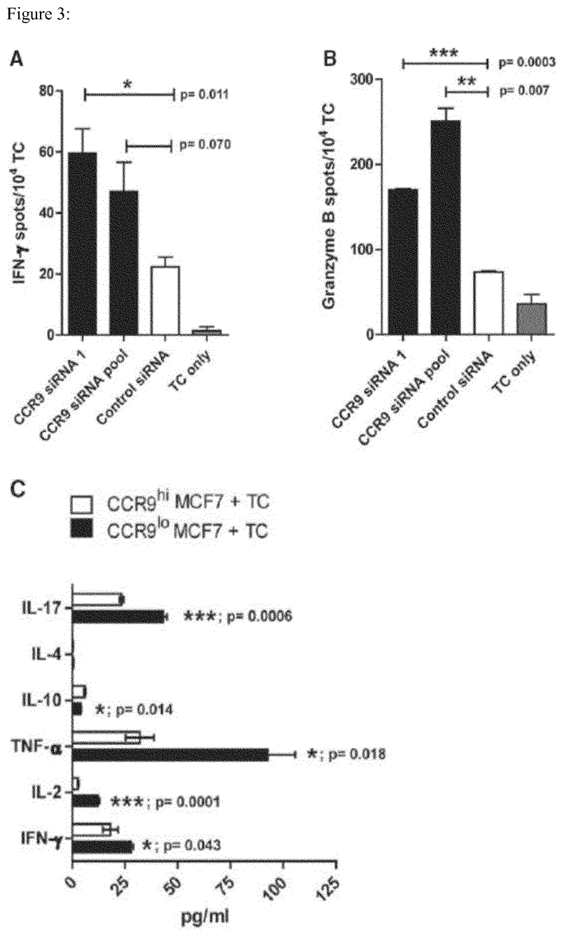

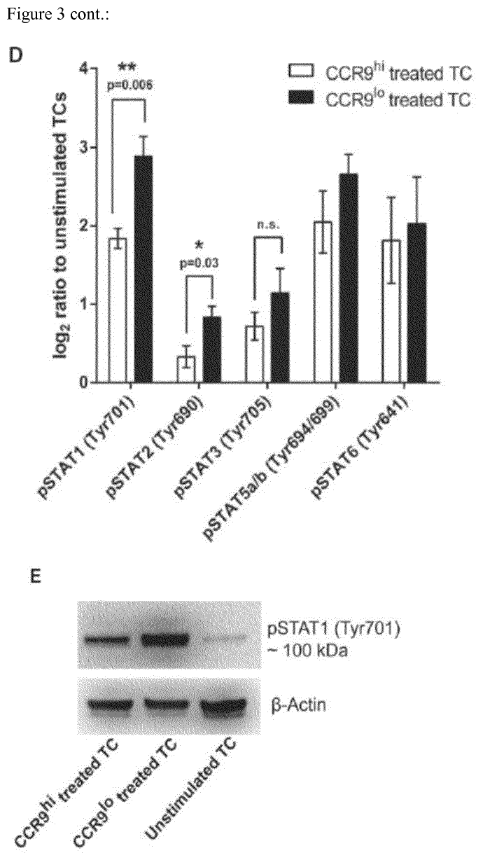

The influence of CCR9 expression on CTL functions was explored. CCR9 knockdown in MCF7 cells significantly increased the secretion of IFN-γ and granzyme B by survivinspecific CTL in response to MCF7 cells (FIGS. 3A and 3B), supporting the increased cytotoxicity observed in the kill assays. To assess whether this correlated with increased TCR activation and signaling, TCR phospho-plex analysis in survivin-specific CTLs was performed after contact with CCR9^{hi} or CCR9^{lo} MCF7 cells. With the exception of some degree of reduced Lck phosphorylation (which was detectable only 5 min after exposure to CCR9^{lo} tumor cells), not any CCR9-dependent changes in TCR signaling was observed. Nevertheless, TCR engagement was found to be necessary for CCR9-mediated immunosuppression as polyclonal T cells failed to secrete higher levels of IFN-γ in response to CCR910 MCF7 cells in the absence of anti-EpCAM×CD3 bi-specific antibody.

One alternative route of T cell activation is the STAT (signal transducer and activator of transcription) family of transcription factors that regulate cytokine expression in T cells (Yu et al, 2009). CCR9 expressed on MCF7 cells significantly inhibited the secretion of the T-helper-1 (Th1) cytokines including tumor necrosis factor-alpha (TNF-α), interleukin-2 (IL-2), and (to a minor extent) of IFN-γ as well as IL-17, while the secretion of IL-10 was slightly but consistently increased (FIG. 3C). Accordingly, a significant increase in STAT1 and STAT2 signaling in survivin-specific T cells upon coculture with CCR910 MCF7 cells was observed, suggesting that anti-tumor type-1 immune response is impeded by tumor-specific CCR9 (FIGS. 3D and 3E).

### Example 3: CCR9 Modulates T-Cell Responses Directly and Independent from Intracellular CCR9 Signalling

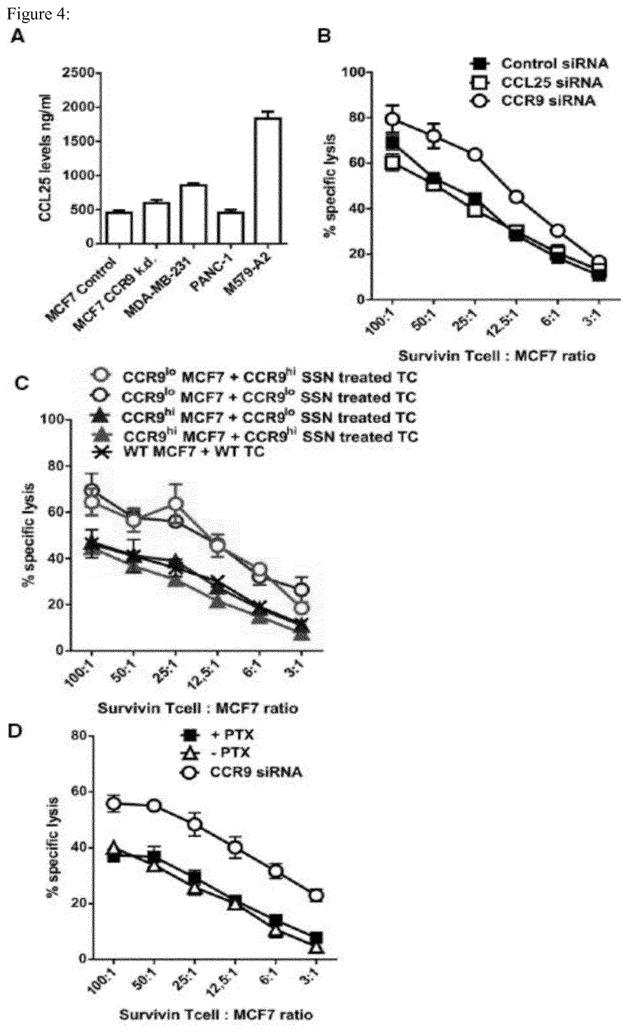

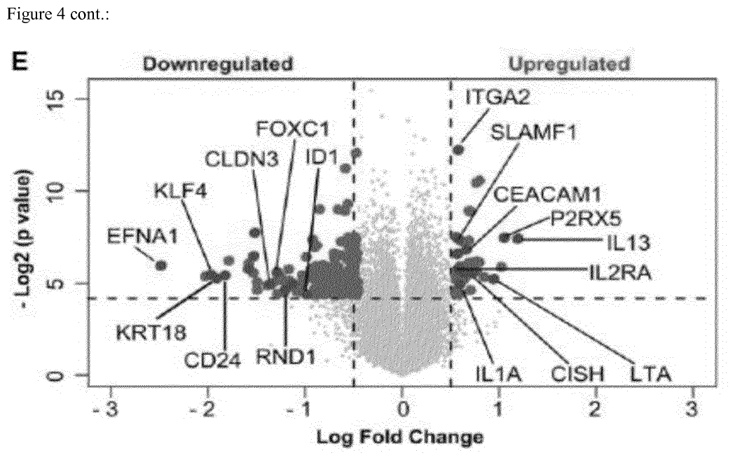

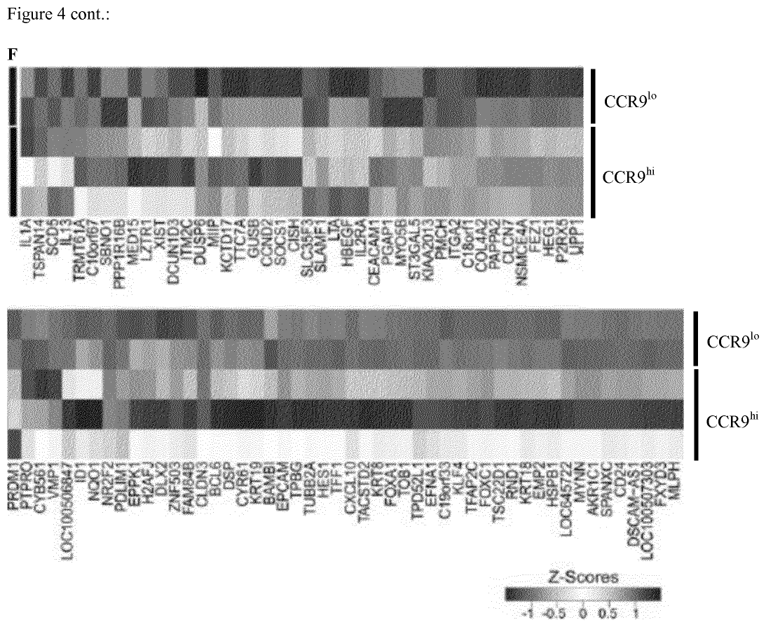

Next, the inventors assessed whether CCR9 expression in breast tumor cells affected T cell recognition directly or indirectly, for example, through CCR9 signaling-mediated increase in secretion of immune-suppressive factors. Since, the C-C chemokine ligand 25 (CCL25) is the only known interacting partner and ligand for CCR9, it was first assessed whether CCL25 was involved in defining CCR9's tolerogenic phenotype. CCL25 was found to be produced by all the studied tumor cell lines, although at varied levels, as determined by ELISA (FIG. 4A). Interestingly, shRNA-mediated stable knockdown of CCR9 did not affect CCL25 production by MCF7 breast cancer cells (FIG. 4A). Next, inhibition of CCL25 using siRNAs (FIG. 4B) or blocking antibody showed no effect on antigen-specific lysis of MCF7 cells, in contrast to the CCR9 knockdown. However, it might still be possible that CCR9 mediates its immunesuppressive effect via other unknown soluble ligands or mediators.

To examine this possibility, survivin-specific T cells were treated with the cell culture supernatants from either the CCR9 siRNA knockdown (CCR9^{lo}) or control (CCR9^{hi}) MCF7 tumor cells overnight and then challenged against CCR9^{hi} or CCR9^{lo} MCF7 cells in the cytotoxicity assay. Against the same tumor target, neither of the supernatant-treated T cells showed any difference in their recognition and lytic capacity. The difference in lysis between the different groups depended upon CCR9's expression on the tumor targets rather than on the T cell treatment (FIG. 4C), hinting to the possibility that T cells can interact directly with CCR9 on tumor cells.

To further assess whether intracellular signaling in tumor cells mediated by the surface-bound CCR9 plays any role in immunosuppression, pertussis toxin (PTX), a G_{αi} inhibitor, was used. Although, pertussis toxin inhibited the migration of CCR9⁺ tumor cells toward CCL25 in a transwell migration assay, proving its effectiveness in blocking CCR9's downstream signaling that is responsible for the chemotaxis, it, however, did not elicit elevated tumor lysis by antigen-specific T cells when compared to the CCR9 gene knockdown (FIG. 4D). This further supported the notion that CCR9-mediated immune suppression on T cells might be independent of its intracellular signaling in the tumor cells and rather affects the T cells directly. Additionally, the inventors evaluated whether CCR9 knockdown influences MHC-I expression on the tumor targets that could possibly explain their impact on T cell recognition and lysis. However, flow cytometric analysis revealed no major alterations in the surface expression of HLA-A2 on the target tumor cell lines upon CCR9 knockdown.

### Example 4: Influence of CCR9 on the Transcriptome of T Cells

To better understand the mode of CCR9-mediated immune suppression on T cells, a broadscale transcriptomics study was performed to compare the changes in the transcriptome of T cells that encounter CCR9^{hi} versus CCR9^{lo} MCF7 tumor cells. Microarray analysis comparing these two T cell populations revealed a list of differentially up- and downregulated genes in CCR9^{lo}-treated T cells, which are represented in the volcano plot of FIG. 4E and in the associated heat map of FIG. 4F. Immune response-related genes such as integrin alpha-2 (ITGA2; Yan et al, 2008), lymphotoxin alpha LTA; (Dobrzanski et al, 2004), interleukin 2 receptor alpha (IL2RA; Pipkin et al, 2010), and cytokine-inducible SH2-containing protein (CISH; Li et al, 2000) were upregulated, whereas genes that inhibit T cell maturation and effector function such as ephrin-A1 (EFNA1; Abouzahr et al, 2006), Kruppel-like factor 4 (KLF4; Wen et al, 2011), inhibitor of DNA binding-1 (ID1; Qi & Sun, 2004), transducer of ERBB2, 1 (TOB1; Tzachanis et al, 2001) were downregulated in T cells encountering CCR9^{lo} tumor cells, which was found to be in accordance with the observed increase in cytotoxicity as shown before. Gene annotation/ontology (GO) analysis of the top upregulated genes revealed an enrichment of genes involved in positive regulation of immune response, while genes involved in lymphocyte maturation and apoptosis were enriched in the list of downregulated genes. The question arose whether these gene signatures observed in T cells upon tumor-specific CCR9 knockdown overlap with gene signatures generally associated with an activated T cell population. Using a publically available gene expression study comparing unstimulated CD8⁺ T cells to CD3/CD28 antibody and IL-2-activated T cells (Wang et al, 2008), we indeed identified overlapping gene signatures in both these studies, suggesting that CCR9 knockdown on tumor cells favors better survival, proliferation, and activation of the encountering T cells.

### Example 5: In Vivo Relevance of CCR9 in Human Cancer

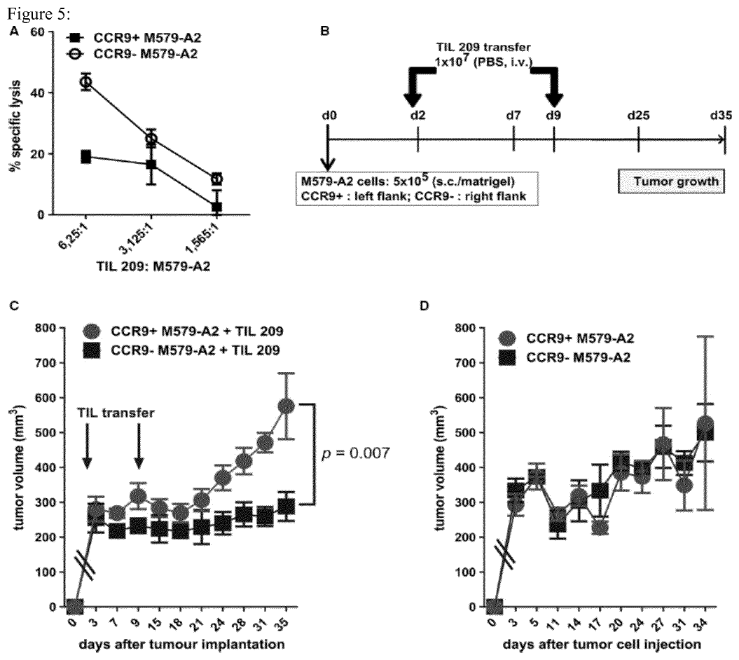

To evaluate the in vivo relevance of CCR9 as a tumor-associated immunosuppressive entity, CCR9 was stably knocked down in the melanoma patient-derived M579-A2 tumor cell culture using CCR9-specific shRNA (shCCR9) or the control non-targeting shRNA (shControl). As expected, stable CCR9 knockdown tumor cell variants were more susceptible to immune lysis by melanoma patient-derived tumor-infiltrating lymphocytes (TIL 209) than their counterparts in the chromium-release cytotoxicity assay (FIG. 5A), with no significant difference noted on the surface HLA-A2 expression upon CCR9 knockdown. For the in vivo analysis, 5×10⁵ cells each of the CCR9⁺ M579-A2 (shControl) and CCR9⁻ M579-A2 (shCCR9) tumor cell lines were subcutaneously implanted in the left and the right flank, respectively, of the NSG immune-deficient mice (scheme in FIG. 5B). These mice then received intravenous injection of 1×10⁷ tumor-infiltrating lymphocytes (TIL 209) at Day 2 and Day 9. As shown in FIG. 5C, CCR9⁻ M579-A2 tumors grew significantly slower than the CCR9⁺ tumors in response to the adoptive T cell transfer, indicating that CCR9 suppresses the anti-tumor activity of the transferred T cells in vivo as well. No difference in the tumor growth kinetic between the CCR9⁺ and the CCR9 tumor cells was observed in mice that received no T cell transfer (FIG. 5D). Taken together, these results suggest an important role for tumor-associated CCR9 as an immune-checkpoint node for application in cancer immunotherapy.

### Example 6: Combination Therapies for Reducing Tumor Resistance

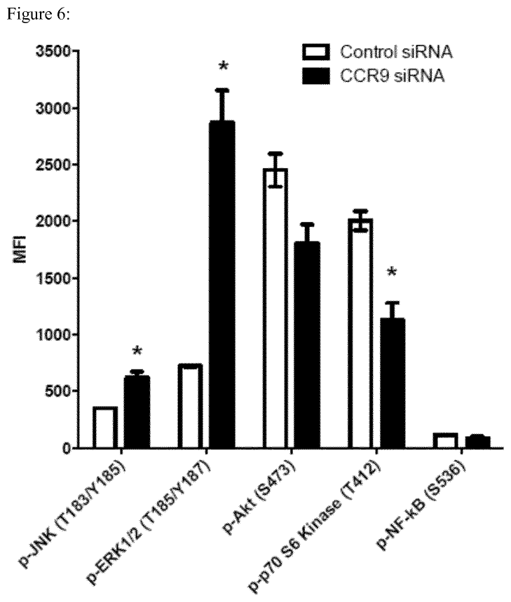

For a rational design of efficient combinatorial therapies for cancer treatment, it is essential to identify whether redundant or divergent signaling pathways underlying the potential immune modulatory function of CCR9 and other immune-checkpoint entities exist, which in a combination therapy are targeted synergistically. In order to identify the signaling pathways involved in CCR9 mediated modulation of tumor cell immune resistance, (intracellular) signaling pathways modulated by (eg, downstream of) CCR9 were characterized using the phosphoprotein analysis of major transcription factors in WT versus CCR9 knockdown MCF7 cells. Knockdown of CCR9 resulted in a significantly reduced signaling via Akt and S6-kinase, whereas a compensatory upregulation in the ERK kinase pathway and in the JNK pathway was noted, indicating their involvement with (eg in the downstream) CCR9 signaling (FIG. 6).

### Example 7: Demonstrating Combination Therapies for the Reduction of Tumor Resistance to Immune Response

To demonstrate the synergy between CCR9-mediated immune suppression and the other relevant signal transduction pathways set forth in the present invention, luciferase-tagged tumor cell lines (based on MCF-7, MDA-MB-231, PANC-1 etc cell lines) are generated analogously to the approach described in the Materials and Methods. Each such luciferase-tagged tumor cell line is then reverse transfected with either control siRNA or CCR9-specific siRNA (Dharmacon, GE healthcare) as described in Khandelwal et al, 2015. Following culture for 72 hours, the cells are incubated with either DMSO alone as control or various concentrations (ranging from 0 nM, 0.1 nM, 10 nM, 100 nM, 1 μM, 10 μM, 100 μM or 1000 μM) of: (i) an inhibitor or antagonist of PI3K-Akt signaling (for example, MK-2206 or MSC-2363318A); (ii) an inhibitor or antagonist of p70S6 kinase signaling (for example, LY-2584702 or LY2780301 or MSC-2363318A); (iii) an activator or agonist of ERK1/2 signaling; or (iv) an activator or agonist of JNK signaling. 1-hour after treatment with the inhibitor/antagonist (or activator/agonists, as applicable), tumor cells are co-cultured with HLA-matched (to the tumor cell line used) T cells (CTLs)—at T-cell to tumor cell ratios of between about 10:1 to 1:1—for an additional 8-10 hours, followed by the Luc-CTL assay readout for assessment of tumor lysis (Khandelwal et al, 2015). For comparison, a sample of the corresponding luciferase-tagged tumor cells is treated solely with CCR9 inhibitor or with the respective modulator of the mentioned pathway. Control experiments—without co-culture with CTLs—are also conducted. The corresponding IC50 values are calculated for each treatment, and the IC50 value of CCR9 inhibitor alone, as well as IC50 of the respective modulator of the aforementioned pathways when used alone, are higher than the IC50 value for treatment of CCR9 inhibitor in combination together with the respective modulator of the aforementioned pathways; thus demonstrating the principle of such CCR9 inhibitor-based combinations as therapies for reducing the resistance of a tumor to an immune response.

Conducting the above experiment in a similar fashion, CCR9 activity in the tumor cells can instead be inhibited by using varying concentrations of an inhibitory anti-CCR9 antibody (or a small-molecule CCR9 inhibitor), and the synergy of such CCR9 inhibition with modulation of the other relevant signal transduction pathways set forth in the present invention can also be demonstrated. Tumor cell lysis can be measured for: (1) the CCR9 inhibitor and for the respective pathway modulator alone; (2) the CCR9 inhibitor in a series of concentrations plus the respective pathway modulator at a set concentration; and (3) the respective modulator in a series of concentrations plus the CCR9 inhibitor at a set concentration. Using such data, a Combination Index can be calculated from the algorithm of Chou & Talala, 1984 (Adv Enzyme Regul; 22:27) using XLfit software (IDBS, Guilford, UK); where Combination Index values of <1, ≈1 and >1 indicate synergisms, additive effect and antagonism, respectively. These data can also be represented using an isobologram. Synergy can also be evaluated by calculation of Bliss independence (Bliss, 1939; Ann Appl Biol 26:585).

Analogous experiments can be conducted to demonstrate the synergy between eg FZD3-mediated immune suppression (or any other negative modulator gene) and the other relevant signal transduction pathways, by using eg FZD3 inhibitors or antagonists, such as eg anti-FZD3 antibodies or eg anti-FZD3 siRNA.

Analogous experiments can be conducted to demonstrate the synergy between eg CXCL9- (or CXCR3-) mediated immune suppression (or any other positive modulator gene) and the other relevant signal transduction pathways, by using eg CXCL9 or CXCR3 activators or agonists.

Materials and Methods

Cell Culture and Reagents

MCF7, MDA-MB-231 (breast cancer), and PANC-1 pancreatic cancer cells were acquired from American Type Cell Culture (Wesel, Germany). MCF7luc cells were generated by electroporation with pEGFP-Luc plasmid and expansion of sorted GFP+ clones in selection medium containing 550 jtg/ml G418 (Gibco, UK). M579-A2 melanoma culture was established from a patient and stably transfected with HLA-A2 expression construct as described before (Machlenkin et al, 2008). For stable CCR9 knockdown, lentiviral particles were produced using the pRSI9-U6-TagRFP-2APuro lentiviral expression vector (Cellecta) that contained either the CCR9-specific shRNA hairpin (ACCGGGCCAGTGGAGGTCTTTGTTCTGTTAATAT TCATAGCAGAACAAGGACCTTCACTGGCTTTT: SEQ ID NO. 5) or control nontargeting shRNA. Viruses were packaged using the psPAX2 and pMD2.G packaging plasmids (Addgene), and tumor cell lines were transduced with the viral particles as per the manufacturer's protocol.

For RNAi screens, CD8+ T cells were isolated from PBMC of healthy donors using CD8 Flow Comp kit (Invitrogen; Karlsruhe, Germany) and activated for 3 days in X-vivo medium (Lonza, Belgium) containing anti-CD3/CD28 activation beads (Dynal, Invitrogen) and 100 U/ml interleukin 2 (IL-2). HLA-A0201-restricted survivin95-104 (clone SK-1)specific CTL clones were generated from PBMC of healthy donors as described (Brackertz et al, 2011). Tumor-infiltrating lymphocytes 412 and 209 microcultures were expanded from an inguinal lymph node of a melanoma patient as described (Dudley et al, 2010). TIL 53 microculture was established from a male patient with poorly differentiated pancreatic adenocarcinoma (PDAC) (Poschke & Offringa, unpublished data) and expanded using the rapid expansion protocol (REP) as described elsewhere (Dudley et al, 2003).

RNAi Screen and Data Analysis

The GPCR-targeting sub-library of the genome-wide siRNA library siGENOME (Dharmacon, GE Healthcare) contained 520 siRNA pools, consisting of four synthetic siRNA duplexes each and was prepared as described (Gilbert et al, 2011). Four RNAi screens were performed in duplicate wells. Positive and negative siRNA controls were distributed into empty wells prior to screening. Reverse siRNA transfection was performed by delivering 0.05 μl of RNAiMAX in 15 μl RPMI (Invitrogen). After 30 min, 3,000 MCF7 cells (screens 1 and 3: MCF7luc, screens 2 and 4: MCF7) in 30 μl DMEM medium (Invitrogen) supplemented with 10% FBS (Invitrogen) were added. Plates were incubated at 37° C. for 24 h, and for screen 2, cells were transiently transfected with a luciferase expression plasmid (pEGFP-Luc) using TransIT-LT1 transfection reagent (Mirius Bio LLC, Madison, USA). 72 h post siRNA transfection, cancer cells were either challenged with CTLs and anti-EpCAM×CD3 bi-specific antibody (0.2 μg/well; screens 1 and 2) or survivin-specific CTLs (screen 3) or left untreated (condition without addition of CTLs and screen 4). Tumor lysis was quantified by analysis of residual luciferase expression in tumor cells (Brown et al, 2005). Screen 1 contained CTLs from one single donor and screen 2 contained CTLs from 2 different donors; one for each technical replicate within the screen. 18 h later, supernatant was removed, cells were lysed, and luciferase measurements (screens 1, 2, and 3) or viability measurements using CellTiterGlo (Promega) (screen 4) were performed as previously described (Muller et al, 2005; Gilbert et al, 2011). Plate reader data from RNAi screens were analyzed using the cellHTS2 package in R/Bioconductor (Boutros et al, 2006). Scores from both conditions, that is, addition of CTLs and without addition of CTLs, were quantile normalized against each other using the aroma.light package in R. Differential scores were calculated using a loess regression fitting.

To reveal high-confidence hits, unsupervised hierarchical clustering of differential score of all genes from all screens was performed using the loess score. In order to robustly identify genes that positively modulate CTL-mediated cytotoxicity and to avoid biases potentially introduced by employing CTLs from different donors and employing genetically engineered as well as unmodified MCF7 cells, we filtered out genes that had a score >2, and <−2 in the condition without addition of CTLs and had a score >0.5, and <−0.5 in the condition with addition of CTLs. Finally, genes scoring in a CTG-based viability screen were filtered out from the candidate list (score <−1.5 and >1.5). Thereby, siRNAs generally affecting cell viability, as determined by intracellular ATP levels, were excluded.

Chromium-Release Cytotoxicity Assay

Tumor cells were transfected with the described siRNAs using RNAiMAX or with pCMV6-AC-His-CCR9 encoding vector and empty control vector (OriGene, Rockville, USA) using TransIT-LT1. 72 h later, transfected cells were harvested for chromium-release cytotoxicity assay as detailed in Supplementary Methods. For CCR9 blockade using pertussis toxin (PTX), 106 tumor cells were incubated with 250 ng/ml of PTX (Sigma Aldrich) for 1 h at 37° C. before labeling with radioactive chromium.

ELISpot Assay

IFN-γ and granzyme B secretion from T cells was determined using ELISpot assay as described by the manufacturer (Mabtech, Nacka Strand, Sweden) and detailed in the Supplementary Methods.

Cytokine and Phospho-Plex Analysis

Cytokines in T cell stimulation cultures were determined with Bio-Plex Pro Assay kit (Biorad, Germany). For phospho-TCR and phospho-STAT analysis, 2×106 survivin-specific TCs were cocultured with the respective target tumor cells at 20:1 ratio for defined time points, then isolated and lysed. Protein lysates were used for 7-plex TCR phosphoprotein kit and phospho-STAT 5-plex kit (Millipore, Billerica, USA) as detailed in the manufacturer's protocol. Measurements were performed using Luminex100 Bio-Plex System (Luminex, Austin, US; see also Supplementary Methods).

Global Gene Expression Analysis

For transcriptomic analysis, 2.5×105 MCF7 cells per group were reverse transfected with control or CCR9 s1 siRNA in 6-well plates and cocultured with 5×106 survivin T cells after 72 h for an additional 12 h. Following co-incubation, TCs were purified using the anti-EpCAM antibody-coated mouse IgG beads (detailed in Supplementary Methods) and total RNA was isolated using the RNeasy Mini kit (Qiagen) as instructed by the manufacturer. Gene expression analysis was performed using the GeneChip Human Genome U133 Plus 2.0 Array (Affymetrix). Gene expression intensity was quantile normalized, and significant differences in the log fold change of gene expression between the CCR9hi- versus the CCR9^{lo} treated TCs were evaluated using the Welch's t-test. Top differentially up- and downregulated genes were plotted as heat maps using heatmap.2 function in R. Expression data can be accessed using the ArrayExpress database (www.ebi.ac.uk/arrayexpress) under accession number E-MTAB-3244. CCR9-induced gene expression signature was compared with a publically available gene expression dataset from a previous study (Wang et al, 2008), which compared CD8+ T cells from the peripheral blood of healthy donors before and after 24 h of activation with anti-CD3/CD28 antibody plus IL-2. The published dataset was retrieved from the Gene Expression Omnibus using the accession code GSE7572 and analyzed using standard methods in R.

In Vivo Experiments

Appropriate approval for animal work was obtained from the regulatory authorities (Regierungsprasidium, Karlsruhe) before the start of the experiment. Four- to six-week-old female NSG mice were ordered from the Animal Core Facility at DKFZ, Heidelberg. Mice were subcutaneously injected with 5×105 cells (in 100 μl of matrigel per injection) of each CCR9-M579-A2 (transduced with CCR9-specific shRNA) and CCR9+M579-A2 (transduced with non-targeting control shRNA) cell lines in the left and the right flank, respectively. Following this, at Day 2 and Day 9, 7 out of the 10 tumor-bearing mice received adoptive transfer of expanded TIL 209 cells intravenously into the tail vein (1×107 cells/100 μl PBS/mouse). The remaining three mice were injected with PBS alone to assess tumor growth in the absence of adoptive TIL transfer. Tumor measurements were performed using a digital caliper (Carl Roth) at the indicated time points, and tumor volume was measured using the formula: volume=height*width*width*(n/3).

Statistical Evaluation

Differences between test and control groups were analyzed by two-sided Student's t-test. In all statistical tests, a P-value <0.05 was considered significant. Statistical difference between the tumor growth curves in vivo was assessed using the unpaired one-sided Mann-Whitney U-test.

### REFERENCES

Abouzahr et al, 2006: Proc Natl Acad Sci USA 103: 1428-1433
Bachmann et al, 1999: Eur J Immunol 29: 291-299
Bellucci et al, 2012: J Clin Investig 122: 2369-2383
Berrien-Elliott et al, 2013: Cancer Res 73: 605-616
Blank et al, 2004: Cancer Res 64: 1140-1145
Boutros et al, 2006: Genome Biol 7: R66
Brackertz et al, 2011: Blood Cancer J 1: e11
Brahmer et al, 2012: New Engl J Med 366: 2455-2465
Brown et al, 2005: J Immunol Methods 297: 39-52
Chambers et al, 2001: Annu Rev Immunol 19: 565-594
Dobrzanski et al, 2004: Cancer Res 64: 406-414
Dudley et al, 2003: J Immunother 26: 332-342
Dudley et al, 2010: Clin Cancer Res 16: 6122-6131
van Elsas et al, 1999: J Exp Med 190: 355-3660
Gilbert et al, 2011: PLoS ONE 6: e28338
Hadeiba et al, 2008: Nat Immunol 9: 1253-1260
Hill & Martins, 2006: Methods 38: 312-316
KhandelwalN et al, 2015: EMBO Molecular Medicine 7: 450-463
Kunkel et al, 2000: J Exp Med 192: 761-768
Li et al, 2000: J Exp Med 191: 985-994
Muller et al, 2005: Nature 436: 871-875
Pipkin et al, 2010: Immunity 32: 79-90
Qi & Sun, 2004: Mol Cell Biol 24: 7313-7323
Rabinovich et al, 2007: Annu Rev Immunol 25: 267-296
Slifka et al, 1999: Nature 401: 76-79
Topalian et al, 2012: New Engl J Med 366: 2443-2454
Tzachanis et al, 2001: Nat Immunol 2: 1174-1182
Uehara et al, 2002: J Immunol 168: 2811-2819
Wang et al, 2008: BMC Med Genomics 1: 53
Weber, 2007: Oncologist 12: 864-872
Wen et al, 2011: Cell Res 21:111701-1710
Woo et al, 2012: Cancer Res 72: 917-927
Yan et al, 2008: J Immunol 181: 4621-4631
Yu et al, 2009: Nat Rev Cancer 9: 798-809
Zitvogel et al, 2006: Nat Rev Immunol 6: 715-727

TABLE 1
Protein/Gene Annotation:

| Gene symbol | Gene Name | Entrez Gene ID |
|---|---|---|
| GHRHR | Growth Hormone Releasing Hormone Receptor | 2692 |
| FLJ31393 (alias for | Olfactory Receptor, Family 7, Subfamily E, | 219445 |
| OR7E5P) | Member 5 Pseudogene | |
| FZD3 | Frizzled class receptor 3 | 7976 |
| OR3A2 | Olfactory Receptor, Family 3, Subfamily A, | 4995 |
| | Member 2 | |
| CXCL3 | Chemokine (C-X-C Motif) Ligand 3 | 2921 |
| GNRHR2 | Gonadotropin-Releasing Hormone (Type 2) | 114814 |
| | Receptor 2 | |
| IL8 | Interleukin 8 | 3576 |
| HTR1D | 5-Hydroxytryptamine (Serotonin) Receptor | 3352 |
| | 1D | |
| CCL23 | Chemokine (C-C Motif) Ligand 23 | 3352 |
| CCL2 | Chemokine (C-C Motif) Ligand 2 | 6347 |
| P2RY11 | Purinergic Receptor P2Y, G-Protein Coupled, 11 | 6347 |
| TRHDE | Thyrotropin-Releasing Hormone Degrading | 29953 |
| | Enzyme | |
| CCR7 | Chemokine (C-C Motif) Receptor 7 | 1236 |
| IL8RA | Interleukin 8 Receptor Alpha | 3577 |
| GPR34 | G Protein-Coupled Receptor 34 | 2857 |
| TACR2 | Tachykinin Receptor 2 | 6865 |
| GPR43 | G protein-coupled receptors 43 (alias FFAR2) | 2867 |
| GCG | Glucagon | 2641 |
| GRK6 | G Protein-Coupled Receptor Kinase 6 | 2870 |
| TAAR8, also known as | Trace Amine Associated Receptor 8 | 83551 |
| TRAR5 | | |
| TAAR9, also known as | Trace Amine Associated Receptor 9 | 134860 |
| TRAR3 | | |
| ENPP2 | Ectonucleotide Pyrophospha- | 5168 |
| | tase/Phosphodiesterase 2 | |
| GRM6 | Glutamate Receptor, Metabotropic 6 | 2916 |
| GRK5 | G Protein-Coupled Receptor Kinase 5 | 2869 |
| OR1G1 | Olfactory Receptor, Family 1, Subfamily G, | |
| | Member 1 | 8390 |
| CCR2 | Chemokine (C-C Motif) Receptor 2 | 729230 |
| ADMR, also known as | Adrenomedullin receptor | 11318 |
| GPR182 | | |
| GRM4 | Glutamate Receptor, Metabotropic 4 | 2914 |
| ADGRV1, also known as | Adhesion G Protein-Coupled Receptor V1 | 84059 |
| MASS1 | | |
| CXCL9 | Chemokine (C-X-C Motif) Ligand 9 | 4283 |
| CXCR3 | Chemokine (C-X-C Motif) Receptor 3 | 2833 |
| | Olfactory Receptor, Family 1, Subfamily D, | |
| OR1D4 | Member 4 | 653166 |
| | Olfactory receptor family 2 subfamily J | 26707 |
| OR2J2 | member 2 | |
| VN1R4 | Vomeronasal 1 receptor 4 | 317703 |
| OR1F1 | Olfactory receptor family 1subfamily F | 4992 |
| | member 1 | |
| CEACAM-6 | Carcinoembryonic antigen related cell adhesion | 4680 |
| | molecule 6 | |
| CD272 | B and T lymphocyte associated | 151888 |

| Gene Symbol | ENSEMBLE information |
|---|---|
| GHRHR | Location: Chromosome 7: 30,938,669-30,993,254 forward |
| | strand. GRCh38: CM000669.2 |
| | Human CCDS set: CCDS5432.1 |
| | UniProtKB identifiers: Q02643 |
| | Ensembl version: ENSG00000106128.18 |
| FLJ31393 | Location: Chromosome 11: 55,979,398-55,980,103 reverse |
| (alias for OR7E5P) | strand. GRCh38: CM000673.2 |
| | Ensembl version: ENSG00000214880.3 |
| FZD3 | Location: Chromosome 8: 28,494,205-28,574,268 forward |
| | strand. GRCh38: CM000670.2 |
| | Human CCDS set: CCDS6069.1 |
| | UniProtKB identifiers: Q9NPG1 |
| | Ensembl version: ENSG00000104290.10 |
| OR3A2 | Location: Chromosome 17: 3,277,899-3,278,974 reverse |
| | strand. GRCh38: CM000679.2 |
| | Human CCDS set: CCDS42233.1 |
| | UniProtKB identifiers: P47893 |
| | Ensembl version: ENSG00000221882.2 |
| CXCL3 | Location: Chromosome 4: 74,036,589-74,038,807 reverse |
| | strand. GRCh38: CM000666.2 |
| | Human CCDS set: CCDS34007.1 |
| | UniProtKB identifiers: P19876 |
| | Ensembl version: ENSG00000163734.4 |
| GNRHR2 | Location: Chromosome 1: 145,919,013 -145,925,341 forward |
| | strand. GRCh38: CM000663.2 |
| | Ensembl version: ENSG00000211451.11 |
| IL8 | Location: Chromosome 1: 145,919,013-145,925,341 forward |
| | strand. GRCh38: CM000663.2 |
| | Ensembl version: ENSG00000211451.11 |
| HTR1D | Location: Chromosome 1: 23,191,895-23,194,729 reverse |
| | strand. GRCh38: CM000663.2 |
| | human CCDS set: CCDS231.1 |
| | UniProtKB identifiers: P28221 |
| | Ensembl version: ENSG00000179546.4 |
| CCL23 | Location: Chromosome 17: 36,013,056-36,017,968 reverse |
| | strand. GRCh38: CM000679.2 |
| | Human CCDS set: CCDS11305.1, CCDS59282.1 |
| | UniProtKB identifiers: P55773 |
| | Ensembl version: ENSG00000274736.4 |
| CCL2 | Location: Chromosome 17: 34,255,218-34,257,203 forward |
| | strand. GRCh38: CM000679.2 |
| | Human CCDS set: CCDS11277.1 |
| | UniProtKB identifiers: P13500 |
| | Ensembl version: ENSG00000108691.9 |
| P2RY11 | Location: Chromosome 19: 10,111,538-10,115,372 forward |
| | strand. GRCh38: CM000681.2 |
| | Human CCDS set: CCDS12226.1 |
| | UniProtKB identifiers: Q96G91 |
| | Ensembl version: ENSG00000244165. |
| TRHDE | Location: Chromosome 12: 72,087,266-72,670,757 forward |
| | strand. GRCh38: CM000674.2 |
| | Human CCDS set: CCDS9004.1 |
| | UniProtKB identifiers: Q9UKU6 |
| | Ensembl version: ENSG00000072657.8 |
| CCR7 | Location: Chromosome 17: 40,553,769-40,565,472 reverse |
| | strand. GRCh38: CM000679.2 |
| | Human CCDS set: CCDS11369.1, CCDS77026.1 |
| | UniProtKB identifiers: P32248 |
| | Ensembl version: ENSG00000126353.3 |
| IL8RA | Location: Chromosome 2: 218,162,845-218,166,995 reverse |
| | strand. GRCh38: CM000664.2 |
| | Human CCDS set: CCDS2409.1 |
| | UniProtKB identifiers: P25024 |
| | Ensembl version: ENSG00000163464.7 |
| GPR34 | Location: Chromosome X: 41,688,973-41,697,277 forward |
| | strand. GRCh38: CM000685.2 |
| | Human CCDS set: CCDS14258.1 |
| | UniProtKB identifiers: Q9UPC5 |
| | Ensembl version: ENSG00000171659.13 |
| TACR2 | Location: Chromosome 10: 69,403,903-69,416,867 reverse |
| | strand. GRCh38: CM000672.2 |
| | Human CCDS set: CCDS7293.1 |
| | UniProtKB identifiers: P21452 |
| | Ensembl version: ENSG00000075073.14 |
| GPR43 | Location: Chromosome 19: 35,443,907-35,451,767 forward |
| | strand. GRCh38: CM000681.2 |
| | Human CCDS set: CCDS12461.1 |
| | UniProtKB identifiers: O15552 |
| | Ensembl version: ENSG00000126262.4 |
| GCG | Location: Chromosome 2: 162,142,873-162,152,404 reverse |
| | strand. GRCh38: CM000664.2 |
| | Human CCDS set: CCDS46439.1 |
| | UniProtK identifiers: P01275 |
| | Ensembl version: ENSG00000115263.14 |
| GRK6 | Location: Chromosome 5: 177,403,204-177,442,901 forward |
| | strand. GRCh38: CM000667.2 |
| | Human CCDS set: CCDS34303.1, CCDS43406.1, |
| | CCDS47348.1 |
| | UniProtKB identifiers: P43250 |
| | Ensembl version: ENSG00000198055.10 |
| TAAR8, also known as TRAR5 | Location: Chromosome 6: 132,552,693-132,553,721 forward |
| | strand. GRCh38: CM000668.2 |
| | Human CCDS set: CCDS5154.1 |
| | UniProtKB identifiers: Q969N4 |
| | Ensembl version: ENSG00000146385.1 |
| TAAR9 also known as TRAR3 | Location: Chromosome 6: 132,538, 290-132,539,336 forward |
| | strand. GRCh38: CM000668.2 |
| | Human CCDS set: CCDS75520.1 |
| | UniProtKB identifiers: Q96RI9 |
| | Ensembl version: ENSG00000237110.2 |
| ENPP2 | Location: Chromosome 8: 119 557 086-119 67 453 reverse |
| | strand. GRCh38: CM000670.2 |
| | Human CCDS set: CCDS34936.1, CCDS47914.1, |
| | CCDS6329.1 |
| | UniProtKB identifiers: Q13822 |
| | Ensembl version: ENSG00000136960.12 |
| GRM6 | Location: Chromosome 5: 178,978,327-178,996,206 reverse |
| | strand. GRCh38: CM000667.2 |
| | Human CCDS set: CCDS4442.1 |
| | UniProtKB identifiers: O15303 |
| | Ensembl version: ENSG00000113262.14 |
| GRK5 | Location: Chromosome 10: 119,207,589-119,459,742 forward |
| | strand. GRCh38: CM000672.2 |
| | Human CCDS set: CCDS7612.1 |
| | UniProtkt identifiers: P34947 |
| | Ensembl version: ENSG00000198873.11 |
| OR1G1 | Location: Chromosome 17: 3,126,584-3,127,581 reverse |
| | strand. GRCh38: CM000679.2 |
| | Human CCDS set: CCDS11020.1 |
| | UniProtKB identifiers: P47890 |
| | Ensembl version: ENSG00000183024.3 |
| CCR2 | Location: Chromosome 3: 46,353,734-46,360,928 forward |
| | strand. GRCh38: CM000665.2 |
| | Human CCDS set: CCDS43078.1, CCDS46813.1 |
| | UniProtKB identifiers: P41597 |
| | Ensembl version: ENSG00000121807.5 |
| ADMR, also known as GPR182 | Location: Chromosome 12: 56,994,446-56,998,441 forward |
| | strand. GRCh38: CM000674.2 |
| | Human CCDS set: CCDS8927.1 |
| | UniProtKB identifiers: O15218 |
| | Ensembl version: ENSG00000166856.2 |
| GRM4 | Location: Chromosome 6: 34,018,645-34,155,622 reverse |
| | strand. GRCh38: CM000668.2 |
| | Human CCDS set: CCDS4787.1, CCDS59010.1, |
| | CCDS59011.1, |
| | UniProtKB identifiers: Q14833 |
| | Ensembl version: ENSG00000124493.13 |
| ADGRV1, also known as MASS1 | Location: Chromosome 5: 90,529,344-91,164,221 forward |
| | strand. GRCh38: CM000667.2 |
| | Human CCDS set: CCDS47246.1 |
| | UniProtKB identifiers: Q8WXG9 |
| | Ensembl version: ENSG00000164199.15 |
| CXCL9 | Location: Chromosome 4: 76,001,275-76,007,488 reverse |
| | strand. GRCh38: CM000666.2 |
| | Human CCDS set: CCDS34014.1 |
| | UniProtKB identifiers: Q07325 |
| | Ensembl version: ENSG00000138755.5 |
| CXCR3 | Location: Chromosome X: 71,615,916-71,618,517 reverse |
| | strand. GRCh38: CM000685.2 |
| | Human CCDS set: CCDS14416.1, CCDS48135.1 |
| | UniProtKB identifiers: P49682 |
| | Ensembl version: ENSG00000186810.7 |
| OR1D4 | Location: Chromosome 17: 3,240,676-3,241,614 forward |
| | strand. GRCh38: CM000679.2 |
| | Ensembl version: EN5G00000255095.1 |
| OR2J2 | Location: Chromosome 6: 29,173,303-29,174,574 forward |
| | strand. GRCh38: CM000668.2 |
| | Human CCDS set: CCDS43434.1 |
| | UniProtKB identifiers: O76002 |
| | Ensembl version: ENSG00000204700.4 |
| VN1R4 | Location: Chromosome 19: 53,266,676-53,267,723 reverse |
| | strand. GRCh38: CM000681.2 |
| | Human CCDS set: CCDS33099.1 |
| | UniProtKB identifiers: Q7Z5H5 |
| | Ensembl version: ENSG00000228567.3 |
| OR1F1 | Location: Chromosome 16: 3,204,247-3,205,188 forward |
| | strand. GRCh38: CM000678.2 |
| | Human CCDS set: CCDS10496.1 |
| | UniProtKB identifiers: O43749 |
| | Ensembl version: ENSG00000168124.2 |
| CEACAM-6 | Location: Chromosome 19: 41,750,977-41,772,208 forward |
| | strand. GRCh38: CM000681.2 |
| | Human CCDS set: CCDS12585.1 |
| | UniProtKB identifiers: P40199 |
| | Ensembl version: ENSG00000086548.8 |
| CD272 | Location: Chromosome 3: 112,463,968-112,499,561 reverse |
| | strand. GRCh38: CM000665.2 |
| | Human CCDS set: CCDS33819.1, CCDS43130.1 |
| | UniProtKB identifiers: Q7Z6A9 |
| | Ensembl version: ENSG00000186265.9 |

1. A combination comprising (a) and (b); or (a) and (c); or (a), (b) and (c); wherein
(a) Is an inhibitor or antagonist of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9,
(b) Is an inhibitor or antagonist of PI3K-Akt signaling and/or an inhibitor or antagonist of p70S6 kinase signaling, and
(c) Is an activator or agonist of ERK1/2 signaling and/or an activator or agonist of JNK signaling.

2. The combination according to claim 1, wherein the combination is a pharmaceutical composition, or is a plurality of pharmaceutical compositions, comprising (a) and (b), or (a) and (c), or (a) and (b) and (c).

3. The combination according to claim 1 or 2, wherein said inhibitor or antagonist of (a) is an inhibitor or antagonist of FZD3.

4. The combination according to any one of claims 1 to 3, comprising (a) and (b), wherein (b) is, an inhibitor or antagonist of PI3K-Akt signaling.

5. The combination according to any one of claims 1 to 5, comprising (a) and (b), wherein (b) is, an inhibitor or antagonist of p70S6 kinases signaling, preferably an inhibitor of S6K.

6. The combination according to any one of claims 1 to 3, comprising (a) and (b), wherein (b) is an mTOR/PI3K dual inhibitor or a dual S6K and Akt inhibitor.

7. The combination according to any one of claims 1 to 3, comprising (a) and (b), wherein (b) comprises MK-2206 (CAS NO: 1032350-13-2).

8. The combination according to any one of claims 1 to 3, comprising (a) and (b), wherein (b) comprises MSC-2363318A (CAS NO: 1379545-95-5).

9. The combination according to any one of claims 1 to 8, wherein (a) is an antibody that binds to the protein that is the target of the inhibitor or antagonist of (a) and inhibits expression of said protein and/or said protein-T-cell interaction and/or said protein cell signaling.

10. The combination according to any one of claims 1 to 8, wherein (a) is a nucleic acid, preferably an siRNA, that inhibits expression of the protein that is the target of the inhibitor or antagonist of (a) and/or said protein-T-cell interaction and/or said protein cell signaling.

11. A method for treating a tumor disease of a patient, wherein the tumor disease is characterized by a resistance of a tumor cell to a T cell mediated immune response of the patient, the method comprising a step of administering to the patient a therapeutically effective amount of the combination according to any one of claims 1 to 12, preferably by administering to the patient a therapeutically effective amount of the components (a) and (b), or (a) and (c), or (a) and (b) and (c) of such combination.

12. The method according to claim 11, wherein the inhibitor or antagonist of (a) is selected from an inhibitor or antagonist of expression of the protein that is the target of the inhibitor or antagonist of (a), an inhibitor or antagonist of said protein signaling, or an inhibitor or antagonist of said protein-T-cell interaction.

13. The method according to claim 11 or 12, wherein said tumor cell is characterized by a detectable cell surface expression of the protein that is the target of the inhibitor or antagonist of (a).

14. The method according to claim 12 or 13, wherein said inhibitor of said protein-T-cell interaction is an inhibitor of said protein mediated STAT1 impairment in T-cells.

15. The combination according to any one of claims 1 to 10, or the method according to any one of claims 11 to 14, wherein said inhibitor or antagonist of (a), said inhibitor or antagonist of PI3K-Akt signaling and/or said inhibitor or antagonist of p70S6 kinase signaling, and/or said activator or agonist of ERK1/2 signaling and/or said activator or agonist of JNK signaling, is a compound selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct, a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

16. The method according to any one of claims 11 to 15, wherein said tumor cell, tumor or tumor disease is characterized by a resistance against T-cell mediated cytotoxicity.

17. The method according to any one of claims 11 to 16, wherein said tumor cell, tumor or tumor disease is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia (or a tumor cell derived therefrom).

18. The method according to any one of claims 11 to 17, wherein said inhibitor or antagonist of (a) is an inhibitor or antagonist of an interaction between a T-cell and the protein that is the target of the inhibitor or antagonist of (a), and said protein-T-cell interaction is a said protein mediated binding of said tumor cell to said T-cell, for example by intermolecular interaction between cell surface expressed said protein on said tumor cell and at least one T-cell component expressed on the cellular surface of said T-cell.

19. The method according to any one of claims 11 to 18, wherein components (a) and (b), or (a) and (c), or (a) and (b) and (c) of said combination are combined by sequential or concomitant administration to a subject suffering from the tumor disease during said treatment, preferably wherein (a) and (b), or (a) and (c), or (a) and (b) and (c) are concomitantly administered during said treatment.

20. A combination comprising (a) and (b), or (a) and (c), or (a) and (b) and (c), wherein
(a) Is an activator or agonist of: CXCL9, CXCR3, GRM4, GRK5, CCR2, ENPP2, GRM6, OR1G1, ADMR, MASS1, or OR1D4,
(b) Is an inhibitor or antagonist of PI3K-Akt signaling and/or an inhibitor or antagonist of p70S6 kinase signaling, and
(c) Is an activator or agonist of ERK1/2 signaling and/or an activator or agonist of JNK signaling.

21. The combination according to claim 20, wherein said activator or agonist of (a) is an activator or agonist of one or other member of the receptor/ligand pair CXCL9 or CXCR3.

22. The combination according to claim 20 or 21 comprising (a) and (b), wherein (b) comprises MK-2206 (CAS NO: 1032350-13-2).

23. The combination according to any one of claims 20 to 22, wherein (a) is an antibody that binds to the protein that is the target of the activator or agonist of (a) and activates expression of said protein and/or said protein-T-cell interaction and/or said protein cell signaling.

24. A method for treating a tumor disease of a patient, wherein the tumor disease is characterized by a resistance of a tumor cell to a T cell mediated immune response of the patient, the method comprising a step of administering to the patient a therapeutically effective amount of the combination according to any one of claims 1 to 9, preferably by administering to the patient a therapeutically effective amount of the components (a) and (b), or (a) and (c), or (a) and (b) and (c) of such combination.

25. The combination according to any one of claims 20 to 23, or the method according to claim 24, wherein said activator or agonist of (a), said inhibitor or antagonist of PI3K-Akt signaling and/or said inhibitor or antagonist of p70S6 kinase signaling, and/or said activator or agonist of ERK1/2 signaling and/or said activator or agonist of JNK signaling, is a compound selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct, a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

26. A method for reducing resistance of a tumor cell to an immune response, the method comprising a step of contacting the tumor cell with a modulator of tumor resistance selected from an
(a) inhibitor or antagonist of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9, or
(b) an activator or agonist of: ENPP2, GRM6, GRK5, OR1G1, CCR2, ADMR, GRM4, MASS1, CXCL9, CXCR3, or OR1D4.

27. The method according to claim 26, wherein the resistance of a tumor cell to an immune response is a resistance of the tumor cell to a T cell mediated immune response.

28. The method according to claim 26 or 27, wherein said modulator of tumor resistance is
(a) An inhibitor or antagonist of expression, protein function, or signaling of a protein selected from: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9, as applicable, or
(b) an activator or agonist of the expression, protein function, or signaling of a protein selected from: CXCL9, CXCR3, GRM4, GRK5, CCR2, ENPP2, GRM6, OR1G1, ADMR, MASS1, or OR1D4, as applicable.

29. The method according to claim 27 or 28, comprising a step of contacting the tumor cell with at least one additional compound effective in the treatment of cancer, preferably wherein the at least one additional compound effective in the treatment of cancer is one or more modulators of tumor resistance selected from
(a) An inhibitor or antagonist of expression, protein function, or signaling of a protein selected from CCR9, GHRHR, FLJ31393, FZD3, OR3A2, CXCL3, GNRHR2, IL8, HTR1D, CCL23, CCL2, P2RY11, TRHDE, CCR7, IL8RA, GPR34, TACR2, GPR43, GCG, GRK6, TRAR5 or TRAR3, and/or
(b) an activator or agonist of the expression, protein function, or signaling of a protein selected from ENPP2, GRM6, GRK5, OR1G1, CCR2, ADMR, GRM4, MASS1, CXCL9, CXCR3, or OR1D4.

30. The method according to claim 27 or 28, comprising a step of contacting the tumor cell with at least one additional compound effective in the treatment of cancer, preferably wherein the at least one additional compound effective in the treatment of cancer is one or more modulators of tumor resistance selected from an inhibitor or antagonist of expression, protein function, or signaling of a protein selected from OR2J2, VN1R4 or OR1F1.

31. The method according to claim 27 or 28, comprising a step of contacting the tumor cell with at least one additional compound effective in the treatment of cancer, preferably wherein the at least one additional compound effective in the treatment of cancer is one or more modulators of tumor resistance selected from an inhibitor or antagonist of expression, protein function, or signaling of a protein selected from CEACAM-6 or CD274.

32. The method according to any one of claims 27 to 31, wherein said tumor cell is characterized by a detectable cell surface expression of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9, as applicable, before contacting the tumor cell with the corresponding modulator of tumor resistance.

33. The method according to claim 28, wherein said inhibitor of said protein-T-cell interaction is an inhibitor of said protein mediated STAT1 impairment in T-cells.

34. A method for treating a tumor disease in a patient, wherein said tumor disease is characterized by a resistance of said tumor against immune responses, the method comprising a step of
(a) Inhibiting or antagonizing in said patient: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9, or
(b) Activating or agonizing: CXCL9, CXCR3, GRM4, GRK5, CCR2, ENPP2, GRM6, OR1G1, ADMR, MASS1, or OR1D4.

35. A method for aiding a patient's immune response against a tumor disease comprising a step of
(a) Inhibiting or antagonizing in said patient: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9, or
(b) Activating or agonizing: CXCL9, CXCR3, GRM4, GRK5, CCR2, ENPP2, GRM6, OR1G1, ADMR, MASS1, or OR1D4.

36. The method according to claim 34 or 35, comprising a step of administering to said patient a therapeutically effective amount of a modulator of tumor resistance selected from an
(a) inhibitor or antagonist of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9, as applicable, or
(b) an activator or agonist of CXCL9, CXCR3, GRM4, GRK5, CCR2, ENPP2, GRM6, OR1G1, ADMR, MASS1, or OR1D4, as applicable.

37. The method according to any one of claims 26 to 36, wherein said inhibitor or antagonist of (a), or said activator or agonist of (b), is a compound is selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct, a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

38. The method according to any one of claims 26 to 37, wherein said tumor cell, tumor or tumor disease is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia.

39. A method for identifying a compound suitable for the treatment of a tumor disease, the method comprising the steps of
(a) Providing a first cell expressing a protein on the cellular surface, wherein the protein is selected from: (x) FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9, or is selected from: (y) CXCL9, CXCR3, GRM4, GRK5, CCR2, ENPP2, GRM6, OR1G1, ADMR, MASS1, or OR1D4,
(b) Providing a candidate compound,
(c) Optionally, providing a second cell which is a cytotoxic T-lymphocyte (CTL), preferably that is capable of immunologically recognizing said first cell, and
(d) Bringing into contact the first cell and the candidate compound and optionally the second cell, and
(e) Determining subsequent to step (d), either or both of
i. expression/function of said protein in said first cell, wherein a differential protein expression in said first cell contacted with the candidate compound compared to said first cell not contacted with said candidate compound indicates that the candidate compound is a compound suitable for the treatment of a tumor disease; and/or
ii. cytotoxicity of said CTL against said first cell, wherein an enhanced cytotoxicity of said CTL against said first cell contacted with the candidate compound compared to the cytotoxicity of said CTL against said first cell not contacted with the candidate compound indicates that the candidate compound is a compound suitable for the treatment of a tumor disease.

40. The method according to claim 39, wherein a reduced protein expression/function of said protein of (x) in said first cell contacted with the candidate compound compared to said first cell not contacted with said candidate compound indicates that the candidate compound is a compound suitable for the treatment of a tumor disease.

41. The method according to claim 39, wherein an increased protein expression/function of said protein of (y) in said first cell contacted with the candidate compound compared to said first cell not contacted with said candidate compound indicates that the candidate compound is a compound suitable for the treatment of a tumor disease.

42. The method according to any one of claims 39 to 41, wherein, said tumor disease or tumor derived cell is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia (or a tumor cell derived therefrom).

43. The method according to any one of claims 39 to 42, wherein said first cell is a cell resistant to cytotoxicity mediated by T-lymphocytes, preferably a tumor derived cell.

44. The method according to any one of claims 39 to 43, wherein said candidate compound is selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPR/Cas9 construct, a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds.

45. A method for diagnosing in a patient a resistance of a tumor disease against T cell mediated immune responses, the method comprising a step of
(a) determining expression of: FZD3, TRHDE, IL8, OR3A2, P2RY11, GHRHR, FLJ31393, CXCL3, GNRHR2, HTR1D, CCL23, CCL2, CCR7, IL8RA, GPR34, TACR2, GPR43, GCG, GRK6, TRAR5, TRAR3 or CCR9 in a tumor cell from the tumor of the patient, wherein a detectable or increased expression of any one of the proteins in the tumor cell compared to a negative control is indicative for a resistance of the tumor disease against T cell mediated immune responses; or
(b) determining expression of: CXCL9, CXCR3, GRM4, GRK5, CCR2, ENPP2, GRM6, OR1G1, ADMR, MASS1, or OR1D4 in a tumor cell from the tumor of the patient, wherein a reduced expression of any one of the proteins in the tumor cell compared to a negative control is indicative for a resistance of the tumor disease against T cell mediated immune responses.

46. The method according to claim 45, comprising a preceding step of obtaining a tumor cell from the patient.

47. The method according to claim 45 or 46, wherein said expression is a cell surface expression of said protein of (a) or (b) on the tumor cell.

48. The method according to any one of claims 45 to 47, wherein, said tumor disease is selected from a liquid or solid tumor, and preferably is breast cancer, ovarian cancer, cancer of the colon and generally the gastro-intestinal tract, lung cancer, e.g., small-cell lung cancer and non-small-cell lung cancer, renal cancer, bladder cancer, prostate cancer, skin cancer like melanoma, head and neck cancer or a tumor disease of the central nervous system, e.g., cervix cancer and, in particular, a brain tumor, more especially astrocytoma, e.g., glioma, or blood cancer such as leukemia.
